(19) United States
(12) Patent Application Publication
(10) Pub. No.: US 2025/0049728 A1
(43) Pub. Date: Feb. 13, 2025

(54) PROCESSES FOR PREPARING LIPID NANOPARTICLES

(71) Applicant: ModernaTX, Inc., Cambridge, MA (US)

(72) Inventors: Chang TIAN, Cambridge, MA (US); Michael H. SMITH, Cambridge, MA (US); Benjamin GELDHOF, Cambridge, MA (US)

(21) Appl. No.: 18/720,092

(22) PCT Filed: Dec. 15, 2022
(86) PCT No.: PCT/US2022/081623

§ 371 Date: Jun. 14, 2024

Related U.S. Application Data
(60) Provisional application No. 63/290,112, filed on Dec. 16, 2021.

(51) Int. Cl. A61K 9/51

(52) U.S. Cl. CPC A61K 9/5123; A61K 9/5192

(57) ABSTRACT

The present disclosure provides processes for controlled high-throughput preparation of lipid nanoparticle with defined parameters, e.g., processes for preparing lipid nanoparticles (LNPs), using a T-Mixer (e.g., a T-Mixer as described herein). The present disclosure also provides lipid nanoparticle solutions (LNP solutions) and lipid nanoparticles (LNPs) being prepared by the processes.

### RELATED APPLICATION

This application claims priority to, and the benefit of, U.S. Application No. 63/290,112, filed Dec. 16, 2021, the entire content of which is incorporated herein by reference.

### BACKGROUND

The effective targeted delivery of biologically active substances such as small molecule drugs, proteins, and nucleic acids represents a continuing medical challenge. In particular, the delivery of nucleic acids to cells is made difficult by the relative instability and low cell permeability of such species.

Lipid-containing nanoparticles or lipid nanoparticles, liposomes, and lipoplexes have proven effective as transport vehicles into cells and/or intracellular compartments for biologically active substances such as small molecule drugs, proteins, and nucleic acids. Yet controlled high throughput process for preparing lipid nanoparticles with defined parameters are and needed.

### SUMMARY

In some aspects, the present disclosure provides a process for preparing lipid nanoparticles (LNPs), comprising mixing a lipid solution with an aqueous buffer solution in a T-Mixer, thereby forming a lipid nanoparticle solution (LNP solution) comprising LNPs, wherein:
the lipid solution is fed to a lipid inlet of the T-Mixer at a lipid inlet back pressure of about 60 psi or less;
the aqueous buffer solution is fed to a buffer inlet of the T-Mixer at a buffer inlet back pressure of about 60 psi or less;
the LNP solution exits the LNP outlet of the T-Mixer at a flow rate of about 500 mL/min or greater; and
the LNPs have an average diameter of about 100 nm or less.

In some aspects, the present disclosure provides a lipid nanoparticle solution (LNP solution) being prepared by a process described herein.

In some aspects, the present disclosure provides a lipid nanoparticle (LNP) being prepared by a process described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a simulation showing the variation of local ethanol mass fraction along the trajectory of lipid dissolved in ethanol in a T-Mixer.

FIG. 1B is a simulation showing the variation of local ethanol mass fraction along the trajectory of lipid dissolved in ethanol in a V-Mixer.

FIG. 1C is a graph showing different ethanol drop time for different flow rate in a 0.5 mm V-Mixer, as a demonstration of decreasing ethanol drop time with increasing flow rate/larger Reynolds number.

FIG. 1D is a diagram showing points on the mesh such as the red highlighted points where massless tracking particles injected onto the ethanol inlet.

FIG. 2 is a graph showing size vs. flow rate through mixer for Benchtop V-Mixer runs.

FIG. 3 is a graph showing size vs. adjusted flow rate through mixer for Benchtop V-Mixer runs.

FIG. 4 is a graph showing size vs. flow rate through mixer for Benchtop T-Mixer runs.

FIG. 5 is a graph showing size vs. adjusted flow rate through mixer for Benchtop T-Mixer runs.

FIG. 6 is a graph showing eLNPs size plotted against ethanol drop time.

FIG. 7A is a bar graph showing counts per mL based on flow cytometry of eLNPs made by 2 mm V-Mixer vs T-Mixer, no in-line static mixer.

FIG. 7B is a flow cam of eLNPs made by 2 mm V-Mixer vs T-Mixer, no in-line static mixer.

FIG. 8 is a bar graph showing counts per mL based on flow cytometry of fLNPs made using GMP skid level mixers.

FIG. 9A is a flow cytometry graph showing no major differences among several samples.

FIG. 9B is a flow cam bar graph showing no major differences among samples. Samples 1-3 are eLNPs made with 4 mm V-Mixer, 3 mm T-Mixer and 4 mm T-Mixer post TFF but before sucrose spike and filtration. Samples 4-6 are Samples 1-3 with sucrose addition of filtration.

FIG. 9C is a graph showing the CZE profiles of samples, with top row unfiltered eLNP and bottom row sucrose added and filtered eLNP. Peaks of 3 mm T eLNPs are on the far right (more positively charged end) due to smaller size of eLNP. Samples 1-3 are eLNPs made with 4 mm V-Mixer, 3 mm T-Mixer and 4 mm T-Mixer post TFF but before sucrose spike and filtration. Samples 4-6 are Samples 1-3 with sucrose addition of filtration.

FIG. 10A and FIG. 10B are a set of graphs showing similar subvisible counting results among several fLNPs.

FIG. 11A and FIG. 11B are a set of graphs showing different V-Mixer prototypes.

### DETAILED DESCRIPTION

### T Mixer and V-Mixer

### T-Mixer

The term “T-Mixer”, as used herein, refers to a mixing device that is configured such that, during mixing, at least two streams are mixed in the device at an angle of about 90° or greater (e.g., about 95° or greater, about 100° or greater, about 105° or greater, about 110° or greater, about 115° or greater, about 120° or greater, about 125° or greater, about 130° or greater, about 135° or greater, about 140° or greater, about 145° or greater, about 150° or greater, about 155° or greater, about 160° or greater, about 165° or greater, about 170° or greater, or about 175° or greater).

In some embodiments, the T-Mixer does not have a mixing chamber.

In some embodiments, the T-Mixer comprises two inlets and an LNP outlet. In some embodiments, the T-Mixer comprises a lipid inlet (via which the stream of the lipid solution enters the T-Mixer), a buffer inlet (via which the aqueous buffer solution enters the T-Mixer), and an LNP outlet (via which the LNP solution exits the T-Mixer.

In some embodiments, the two inlets (e.g., the lipid inlet and the buffer) are positioned at an angle of about 90° or greater (e.g., about 95° or greater, about 100° or greater, about 105° or greater, about 110° or greater, about 115° or greater, about 120° or greater, about 125° or greater, about 130° or greater, about 135° or greater, about 140° or greater, about 145° or greater, about 150° or greater, about 155° or greater, about 160° or greater, about 165° or greater, about 170° or greater, or about 175° or greater). In some embodiments, the two inlets (e.g., the lipid inlet and the buffer) are positioned at an angle of about 180±30°, about 180±25°, about 180±20°, about 180±15°, about 180±10°, about 180±9°, about 180±8°, about 180±7°, about 180±6°, about 180±5°, about 180±4°, about 180±3°, about 180±2°, or about 180±1 (e.g., about) 180°.

In some embodiments, the two inlets (e.g., the lipid inlet and the buffer) meets at a joint that further connects the LNP outlet. In some embodiments, the stream of the lipid solution and the stream of the aqueous buffer solution meet at the joint between the lipid inlet, the buffer inlet, and the LNP outlet.

In some embodiments, the stream of the lipid solution and the stream of the aqueous buffer solution meet at an angle of about 90° or greater (e.g., about 95° or greater, about 100° or greater, about 105° or greater, about 110° or greater, about 115° or greater, about 120° or greater, about 125° or greater, about 130° or greater, about 135° or greater, about 140° or greater, about 145° or greater, about 150° or greater, about 155° or greater, about 160° or greater, about 165° or greater, about 170° or greater, or about 175° or greater). In some embodiments, the stream of the lipid solution and the stream of the aqueous buffer solution meet at an angle of about 180±30°, about 180±25°, about 180±20°, about 180±15°, about 180±10°, about 180±9°, about 180±8°, about 180±7°, about 180±6°, about 180±5°, about 180±4°, about 180±3°, about 180±2°, or about 180±1 (e.g., about) 180°.

In some embodiments, the two inlets form multiple bends and meet at a joint that further connects the LNP outlet. In other words, the two inlets can each independently form flow paths that bend one or more times before meeting at the LNP outlet. In some embodiments, the T-mixer can be assembled from coupling a plurality of layers (e.g., 2 layers, 3 layers, or 4 layers) together. In some embodiments, the two inlets follow a straight path and converge at a joint that further connects the LNP outlet. In some embodiments, the T-mixer is formed from drilling holes into one or more pieces of material to form bores for the two inlets and the LNP outlet.

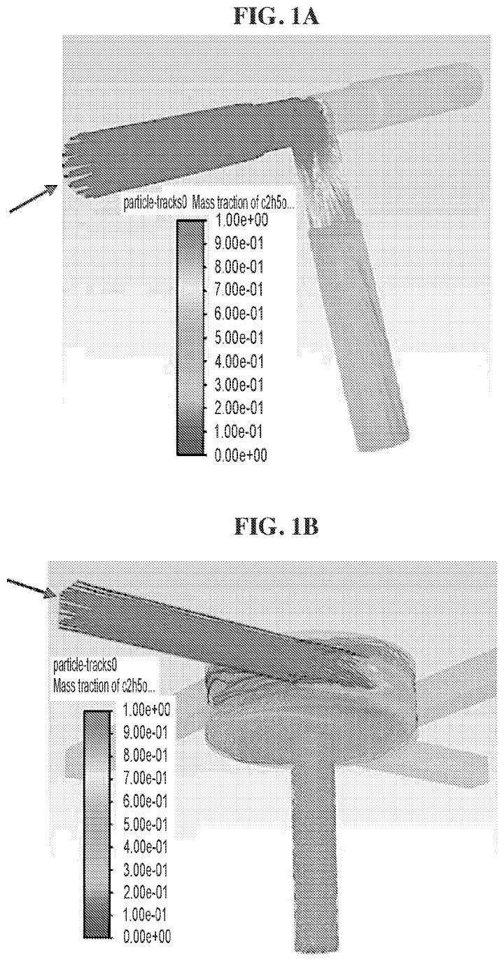

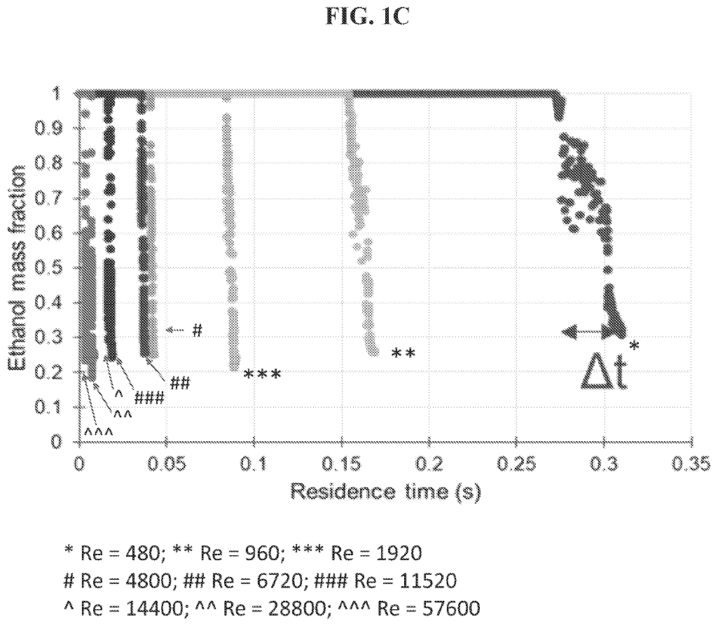

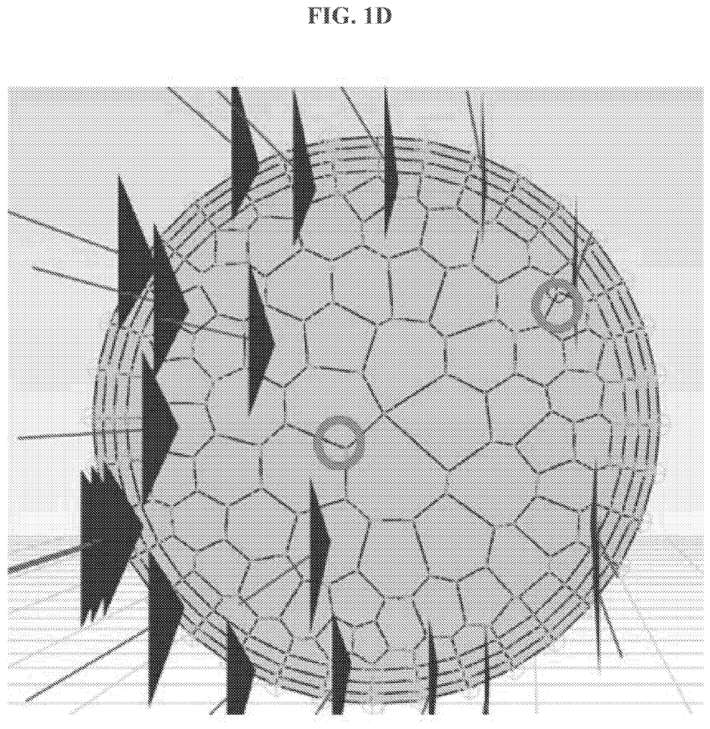

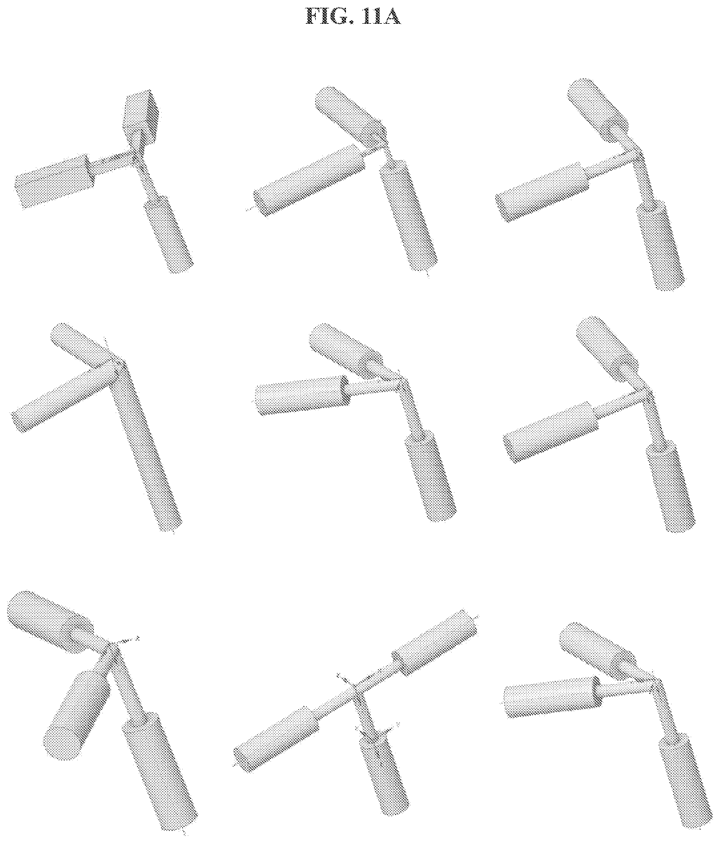

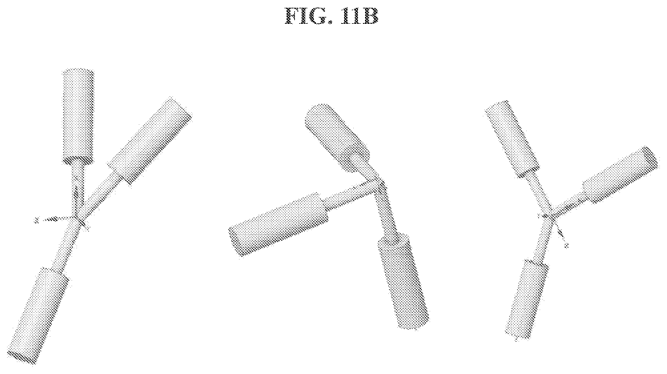

In some embodiments, the V-Mixer is substantially the same as the mixers described in FIG. 1A and FIGS. 11A and 11B.

### V-Mixer

The term “V-Mixer”, as used herein, refers to a mixing device that is configured such that, during mixing, at least two streams are mixed in the device at an angle less than about 90° (e.g., less than about 85°, less than about 80°, less than about 75°, less than about 70°, less than about 65°, less than about 60°, less than about 55°, less than about 50°, less than about 45°, less than about 40°, less than about 35°, less than about 30°, less than about 25°, less than about 20°, less than about 15°, or less than about) 10°.

In some embodiments, the mixing device is configured to have the lipid solution and the aqueous solution tangentially introduced into the cylindrical mixing chamber. In some embodiments, the V-Mixer comprises two inlets (e.g., two, three, or four inlets) and an LNP outlet. In some embodiments, the V-Mixer comprises a lipid inlet (e.g., one or two lipid inlets), a buffer inlet (e.g., one or two buffer inlets), and an LNP outlet.

In some embodiments, the V-Mixer further comprises a mixing chamber (e.g., a cylindrical chamber) that connects the inlets and the outlet. In some embodiments, the V-Mixer is configured such that, during mixing, a stream of a transient mixture flows inside the mixing chamber before exiting the mixing chamber vis the LNP outlet. In some embodiments, the mixing is substantially complete before the stream of the transient mixture exits the mixing chamber.

In some embodiments, the stream of the lipid solution enters the mixing chamber via the lipid inlet. In some embodiments, the stream of the lipid solution and the stream of the transient mixture meet at an angle less than about 90° (e.g., less than about 85°, less than about 80°, less than about 75°, less than about 70°, less than about 65°, less than about 60°, less than about 55°, less than about 50°, less than about 45°, less than about 40°, less than about 35°, less than about 30°, less than about 25°, less than about 20°, less than about 15°, or less than about) 10°.

In some embodiments, the stream of the aqueous buffer solution enters the mixing chamber via the buffer inlet. In some embodiments, the stream of the aqueous buffer solution and the stream of the transient mixture meet at an angle less than about 90° (e.g., less than about 85°, less than about 80°, less than about 75°, less than about 70°, less than about 65°, less than about 60°, less than about 55°, less than about 50°, less than about 45°, less than about 40°, less than about 35°, less than about 30°, less than about 25°, less than about 20°, less than about 15°, or less than about 10°).

In some embodiments, the V-Mixer is substantially the same as the mixer described in in FIG. 1B.

### Processes for Preparing LNPs

In some aspects, the present disclosure provides a process for preparing lipid nanoparticles (LNPs), comprising mixing a lipid solution with an aqueous buffer solution in a T-Mixer, thereby forming a lipid nanoparticle solution (LNP solution) comprising LNPs, wherein:
the lipid solution is fed to a lipid inlet of the T-Mixer at a lipid inlet back pressure of about 60 psi or less;
the aqueous buffer solution is fed to a buffer inlet of the T-Mixer at a buffer inlet back pressure of about 60 psi or less;
the LNP solution exits the LNP outlet of the T-Mixer at a flow rate of about 500 mL/min or greater; and
the LNPs have an average diameter of about 100 nm or less.

In some embodiments, the lipid inlet back pressure of the lipid solution in the T-Mixer is lower as compared to the lipid inlet back pressure of a comparable process using a V-Mixer.

In some embodiments, the buffer inlet back pressure of the lipid solution in the T-Mixer is lower as compared to the buffer inlet back pressure of a comparable process using a V-Mixer.

In some embodiments, the diameter of the T-Mixer is greater than the diameter of a V-Mixer used in a comparable process.

In some embodiments, the LNP flow rate of the LNP solution exits the T-Mixer is greater than the LNP flow rate of a comparable process using a V-Mixer.

In some embodiments, the average diameter of the LNPs is less than the average diameter of the LNPs being prepared by a comparable process using a V-Mixer.

### Lipid Solution

In some embodiments, the processes comprise providing a lipid solution.

In some embodiments, the lipid solution is substantially free of any nucleic acid (e.g., RNA).

In some embodiments, the lipid solution is free of any nucleic acid (e.g., RNA).

In some embodiments, the lipid solution may comprise an ionizable lipid.

In some embodiments, the lipid solution

In some embodiments, the lipid solution further comprises a structural lipid.

In some embodiments, the lipid solution further comprises a phospholipid.

In some embodiments, the lipid solution further comprises a structural lipid and a phospholipid.

In some embodiments, the lipid solution further comprises a PEG lipid.

In some embodiments, the lipid solution further comprises a structural lipid, a phospholipid, and a PEG lipid.

In some embodiments, the lipid solution further comprises an organic solvent.

In some embodiments, the organic solvent is a C₁-C₆ alcohol.

In some embodiments, the C₁-C₆ alcohol is ethanol.

In some embodiments, the lipid solution may comprise the ionizable lipid at a concentration of greater than about 0.01 mg/mL, 0.05 mg/mL, 0.06 mg/mL, 0.07 mg/mL, 0.08 mg/mL, 0.09 mg/mL, 0.1 mg/mL, 0.15 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1.0 mg/mL. In some embodiments, the lipid solution may comprise a ionizable lipid at a concentration ranging from about 0.01-1.0 mg/mL, 0.01-0.9 mg/mL, 0.01-0.8 mg/mL, 0.01-0.7 mg/mL, 0.01-0.6 mg/mL, 0.01-0.5 mg/mL, 0.01-0.4 mg/mL, 0.01-0.3 mg/mL, 0.01-0.2 mg/mL, 0.01-0.1 mg/mL, 0.05-1.0 mg/mL, 0.05-0.9 mg/mL, 0.05-0.8 mg/mL, 0.05-0.7 mg/mL, 0.05-0.6 mg/mL, 0.05-0.5 mg/mL, 0.05-0.4 mg/mL, 0.05-0.3 mg/mL, 0.05-0.2 mg/mL, 0.05-0.1 mg/mL, 0.1-1.0 mg/mL, 0.2-0.9 mg/mL, 0.3-0.8 mg/mL, 0.4-0.7 mg/mL, or 0.5-0.6 mg/mL. In some embodiments, the lipid solution may comprise an ionizable lipid at a concentration up to about 5.0 mg/mL, 4.0 mg/mL, 3.0 mg/mL, 2.0 mg/mL, 1.0 mg/mL, 0.09 mg/mL, 0.08 mg/mL, 0.07 mg/mL, 0.06 mg/mL, or 0.05 mg/mL.

In some embodiments, the lipid solution may comprise an ionizable lipid. In some embodiments, the lipid solution may comprise the ionizable lipid at a concentration of greater than about 0.1 mg/mL, 0.5 mg/mL, 0.6 mg/mL, 0.7 mg/mL, 0.8 mg/mL, 0.9 mg/mL, 1.0 mg/mL, 1.5 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10 mg/mL, 11 mg/mL, 12 mg/mL, 13 mg/mL, 14 mg/mL, 15 mg/mL, 20 mg/mL, 25 mg/mL or 30 mg/mL. In some embodiments, the lipid solution may comprise a ionizable lipid at a concentration ranging from about 0.1-20.0 mg/mL, 0.1-19 mg/mL, 0.1-18 mg/mL, 0.1-17 mg/mL, 0.1-16 mg/mL, 0.1-15 mg/mL, 0.1-14 mg/mL, 01-13 mg/mL, 0.1-12 mg/mL, 0.1-11 mg/mL, 0.5-10.0 mg/mL, 0.5-9 mg/mL, 0.5-8 mg/mL, 0.5-7 mg/mL, 0.5-6 mg/mL, 0.5-5.0 mg/mL, 0.5-4 mg/mL, 0.5-3 mg/mL, 0.5-2 mg/mL, 0.5-1 mg/mL, 1-20 mg/mL, 1-15 mg/mL, 1-12 mg/mL, 1-10 mg/mL, or 1-8 mg/mL. In some embodiments, the lipid solution may comprise an ionizable lipid at a concentration up to about 30 mg/mL, 25, mg/mL, 20 mg/mL, 18 mg/mL, 16 mg/mL, 15 mg/mL, 14 mg/mL, 12 mg/mL, 10 mg/mL, 8 mg/mL, 6 mg/mL, 5.0 mg/mL, 4.0 mg/mL, 3.0 mg/mL, 2.0 mg/mL, 1.0 mg/mL, 0.09 mg/mL, 0.08 mg/mL, 0.07 mg/mL, 0.06 mg/mL, or 0.05 mg/mL.

In some embodiments, the lipid solution comprises an ionizable lipid in an aqueous buffer and/or organic solution.

In some embodiments, the lipid nanoparticle solution further comprises a buffering agent and/or a salt. Exemplary suitable buffering agents include, but are not limited to, ammonium sulfate, sodium bicarbonate, sodium citrate, sodium acetate, potassium phosphate, sodium phosphate, HEPES, and the like. In some embodiments, the lipid solution comprises a buffering agent at a concentration ranging from about 0.1-100 mM, from about 0.5-90 mM, from about 1.0-80 mM, from about 2-70 mM, from about 3-60 mM, from about 4-50 mM, from about 5-40 mM, from about 6-30 mM, from about 7-20 mM, from about 8-15 mM, from about 9-12 mM. In some embodiments, the lipid solution comprises a buffering agent at a concentration of or greater than about 0.1 mM, 0.5 mM, 1 mM, 2 mM, 4 mM, 6 mM, 8 mM, 10 mM, 15 mM, 20 mM, 25 mM, 30 mM, 35 mM, 40 mM, 45 mM, or 50 mM. Exemplary suitable salts include, but are not limited to, potassium chloride, magnesium chloride, sodium chloride, and the like.

In some embodiments, the lipid solution comprises a salt at a concentration ranging from about 1-500 mM, from about 5-400 mM, from about 10-350 mM, from about 15-300 mM, from about 20-250 mM, from about 30-200 mM, from about 40-190 mM, from about 50-180 mM, from about 50-170 mM, from about 50-160 mM, from about 50-150 mM, or from about 50-100 mM. In some embodiments, the lipid nanoparticle solution comprises a salt at a concentration of or greater than about 1 mM, 5 mM, 10 mM, 20 mM, 30 mM, 40 mM, 50 mM, 60 mM, 70 mM, 80 mM, 90 mM, or 100 mM.

In some embodiments, the lipid solution has a pH ranging from about 4.5 to about 7.0, about 4.6 to about 7.0, about 4.8 to about 7.0, about 5.0 to about 7.0, about 5.5 to about 7.0, about 6.0 to about 7.0, about 6.0 to about 6.9, about 6.0 to about 6.8, about 6.0 to about 6.7, about 6.0 to about 6.6, about 6.0 to about 6.5. In some embodiments, a suitable lipid solution may have a pH of or no greater than 4.5, 4.6, 4.7, 4.8, 4.9, 5.0, 5.2, 5.4, 5.6, 5.8, 6.0, 6.1, 6.2, 6.3, 6.4, 6.5, 6.6, 6.7, 6.8, 6.9, and 7.0.

### Lipid Inlet Back Pressure

In some embodiments, the lipid solution is fed to the lipid inlet of the T-Mixer at a lipid inlet back pressure of about 10 psi or greater, about 11 psi or greater, about 12 psi or greater, about 13 psi or greater, about 14 psi or greater, about 15 psi or greater, about 16 psi or greater, about 17 psi or greater, about 18 psi or greater, about 19 psi or greater, about 20 psi or greater, about 21 psi or greater, about 22 psi or greater, about 23 psi or greater, about 24 psi or greater, about 25 psi or greater, about 26 psi or greater, about 27 psi or greater, about 28 psi or greater, about 29 psi or greater, about 30 psi or greater, about 32 psi or greater, about 34 psi or greater, about 36 psi or greater, about 38 psi or greater, about 40 psi or greater, about 42 psi or greater, about 44 psi or greater, about 46 psi or greater, about 48 psi or greater, about 50 psi or greater, about 52 psi or greater, about 54 psi or greater, about 56 psi or greater, or about 58 psi or greater.

In some embodiments, the lipid solution is fed to the lipid inlet of the T-Mixer at a lipid inlet back pressure of about 10 psi or less, about 11 psi or less, about 12 psi or less, about 13 psi or less, about 14 psi or less, about 15 psi or less, about 16 psi or less, about 17 psi or less, about 18 psi or less, about 19 psi or less, about 20 psi or less, about 21 psi or less, about 22 psi or less, about 23 psi or less, about 24 psi or less, about 25 psi or less, about 26 psi or less, about 27 psi or less, about 28 psi or less, about 29 psi or less, about 30 psi or less, about 32 psi or less, about 34 psi or less, about 36 psi or less, about 38 psi or less, about 40 psi or less, about 42 psi or less, about 44 psi or less, about 46 psi or less, about 48 psi or less, about 50 psi or less, about 52 psi or less, about 54 psi or less, about 56 psi or less, about 58 psi or less, or about 60 psi or less.

Combinations of the above-recited ranges for the lipid inlet back pressure are also contemplated (e.g., about 15 psi to about 50 psi, about 15 psi to about 55 psi, about 15 psi to about 60 psi, about 10 psi to about 60 psi, about 15 psi to about 60 psi, or about 20 psi to about 60 psi.)

In some embodiments, the lipid solution is fed to the lipid inlet of the T-Mixer at a lipid inlet back pressure of about 10 psi, about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 32 psi, about 34 psi, about 36 psi, about 38 psi, about 40 psi, about 42 psi, about 44 psi, about 46 psi, about 48 psi, about 50 psi, about 52 psi, about 54 psi, about 56 psi, or about 58 psi, or about 60 psi.

### Aqueous Buffer Solution

In some embodiments, the aqueous buffer solution comprises a buffering agent.

In some embodiments, the aqueous buffer solution is substantially free of any nucleic acid (e.g., RNA).

In some embodiments, the aqueous buffer solution is free of any nucleic acid (e.g., RNA).

In some embodiments, a suitable solution may further comprise one or more buffering agent and/or a salt. Exemplary suitable buffering agents include, but are not limited to, ammonium sulfate, sodium bicarbonate, sodium citrate, sodium acetate, potassium phosphate, sodium phosphate, HEPES, and the like.

In some embodiments, the aqueous buffer solution comprises a buffering agent at a concentration ranging from about 0.1-100 mM, from about 0.5-90 mM, from about 1.0-80 mM, from about 2-70 mM, from about 3-60 mM, from about 4-50 mM, from about 5-40 mM, from about 6-30 mM, from about 7-20 mM, from about 8-15 mM, from about 9-12 mM.

In some embodiments, the aqueous buffer solution comprises a buffering agent at a concentration of or greater than about 0.1 mM, 0.5 mM, 1 mM, 2 mM, 4 mM, 6 mM, 8 mM, 10 mM, 15 mM, 20 mM, 25 mM, 30 mM, 35 mM, 40 mM, 45 mM, or 50 mM. Exemplary suitable salts include, but are not limited to, potassium chloride, magnesium chloride, sodium chloride, and the like. In some embodiments, the aqueous buffer solution comprises a salt at a concentration ranging from about 1-500 mM, from about 5-400 mM, from about 10-350 mM, from about 15-300 mM, from about 20-250 mM, from about 30-200 mM, from about 40-190 mM, from about 50-180 mM, from about 50-170 mM, from about 50-160 mM, from about 50-150 mM, or from about 50-100 mM.

In some embodiments, the nucleic acid solution comprises a salt at a concentration of or greater than about 1 mM, 5 mM, 10 mM, 20 mM, 30 mM, 40 mM, 50 mM, 60 mM, 70 mM, 80 mM, 90 mM, or 100 mM.

In some embodiments, the aqueous buffer solution has a pH ranging from about 4.5 to about 7.0, about 4.6 to about 7.0, about 4.8 to about 7.0, about 5.0 to about 7.0, about 5.5 to about 7.0, about 6.0 to about 7.0, about 6.0 to about 6.9, about 6.0 to about 6.8, about 6.0 to about 6.7, about 6.0 to about 6.6, about 6.0 to about 6.5. In some embodiments, a suitable aqueous buffer solution may have a pH of or no greater than 4.5, 4.6, 4.7, 4.8, 4.9 5.0, 5.2, 5.4, 5.6, 5.8, 6.0, 6.1, 6.2, 6.3, 6.4, 6.5, 6.6, 6.7, 6.8, 6.9, and 7.0.

### Buffer Inlet Back Pressure

In some embodiments, the aqueous buffer solution is fed to an buffer inlet of the T-Mixer at a buffer inlet back pressure of about 10 psi or greater, about 11 psi or greater, about 12 psi or greater, about 13 psi or greater, about 14 psi or greater, about 15 psi or greater, about 16 psi or greater, about 17 psi or greater, about 18 psi or greater, about 19 psi or greater, about 20 psi or greater, about 21 psi or greater, about 22 psi or greater, about 23 psi or greater, about 24 psi or greater, about 25 psi or greater, about 26 psi or greater, about 27 psi or greater, about 28 psi or greater, about 29 psi or greater, about 30 psi or greater, about 32 psi or greater, about 34 psi or greater, about 36 psi or greater, about 38 psi or greater, about 40 psi or greater, about 42 psi or greater, about 44 psi or greater, about 46 psi or greater, about 48 psi or greater, about 50 psi or greater, about 52 psi or greater, about 54 psi or greater, about 56 psi or greater, or about 58 psi or greater.

In some embodiments, the aqueous buffer solution is fed to an buffer inlet of the T-Mixer at a buffer inlet back pressure of about 10 psi or less, about 11 psi or less, about 12 psi or less, about 13 psi or less, about 14 psi or less, about 15 psi or less, about 16 psi or less, about 17 psi or less, about 18 psi or less, about 19 psi or less, about 20 psi or less, about 21 psi or less, about 22 psi or less, about 23 psi or less, about 24 psi or less, about 25 psi or less, about 26 psi or less, about 27 psi or less, about 28 psi or less, about 29 psi or less, about 30 psi or less, about 32 psi or less, about 34 psi or less, about 36 psi or less, about 38 psi or less, about 40 psi or less, about 42 psi or less, about 44 psi or less, about 46 psi or less, about 48 psi or less, about 50 psi or less, about 52 psi or less, about 54 psi or less, about 56 psi or less, about 58 psi or less, or about 60 psi or less.

Combinations of the above-recited ranges for the buffer inlet back pressure are also contemplated (e.g., about 15 psi to about 50 psi, about 15 psi to about 55 psi, about 15 psi to about 60 psi, about 10 psi to about 60 psi, about 15 psi to about 60 psi, or about 20 psi to about 60 psi.)

In some embodiments, the aqueous buffer solution is fed to an buffer inlet of the T-Mixer at a buffer inlet back pressure of about 10 psi, about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 32 psi, about 34 psi, about 36 psi, about 38 psi, about 40 psi, about 42 psi, about 44 psi, about 46 psi, about 48 psi, about 50 psi, about 52 psi, about 54 psi, about 56 psi, or about 58 psi, or about 60 psi.

### Transient Mixture and Organic Solvent Drop Time

In some embodiments, upon mixing, the lipid solution and the aqueous buffer solution form a transient mixture, wherein the concentration of the organic solvent in the transient mixture reduces over an organic solvent drop time, thereby forming the LNP solution.

In some embodiments, the organic solvent drop time is about 0.5 second or less, about about 0.45 second or less, about 0.4 second or less, about 0.35 second or less, about 0.3 second or less, about 0.25 second or less, about 0.2 second or less, about 0.15 second or less, about 0.1 second or less, about 0.09 second or less, about 0.08 second or less, about 0.07 second or less, about 0.06 second or less, about 0.05 second or less, about 0.04 second or less, about 0.03 second or less, about 0.02 second or less, or about 0.01 second or less.

In some embodiments, the organic solvent drop time is about 0.45 second or greater, about 0.4 second or greater, about 0.35 second or greater, about 0.3 second or greater, about 0.25 second or greater, about 0.2 second or greater, about 0.15 second or greater, about 0.1 second or greater, about 0.09 second or greater, about 0.08 second or greater, about 0.07 second or greater, about 0.06 second or greater, about 0.05 second or greater, about 0.04 second or greater, about 0.03 second or greater, about 0.02 second or greater, or about 0.005 second or greater.

Combinations of the above-recited ranges for the LNPs average diameter are also contemplated (e.g., about 0.005 second to about 0.4 second, about 0.005 second to about 0.45 second, about 0.005 second to about 0.5 second, about 0.01 second to about 0.5 second, or about 0.02 second to about 0.5 second)

In some embodiments, the organic solvent drop time is about 0.5 second, about 0.45 second, about 0.4 second, about 0.35 second, about 0.3 second, about 0.25 second, about 0.2 second, about 0.15 second, about 0.1 second, about 0.09 second, about 0.08 second, about 0.07 second, about 0.06 second, about 0.05 second, about 0.04 second, about 0.03 second, about 0.02 second, about 0.01 second, or about 0.005 second.

### Diameter the Mixer

It is understood that the diameter of a mixer, as used herein, refers to the diameter of the outlet of the mixer.

In some embodiments, the T-Mixer has a diameter of about 0.1 mm. In some embodiments, the T-Mixer has a diameter of about 0.3 mm. In some embodiments, the T-Mixer has a diameter of about 0.5 mm. In some embodiments, the T-Mixer has a diameter of about 0.7 mm. In some embodiments, the T-Mixer has a diameter of about 1 mm. In some embodiments, the T-Mixer has a diameter of about 1.5 mm. In some embodiments, the T-Mixer has a diameter of about 2 mm. In some embodiments, the T-Mixer has a diameter of about 2.5 mm. In some embodiments, the T-Mixer has a diameter of about 3 mm. In some embodiments, the T-Mixer has a diameter of about 3.5 mm. In some embodiments, the T-Mixer has a diameter of about 4 mm. In some embodiments, the T-Mixer has a diameter of about 4.5 mm. In some embodiments, the T-Mixer has a diameter of about 5 mm. In some embodiments, the T-Mixer has a diameter of about 6 mm. In some embodiments, the T-Mixer has a diameter of about 7 mm. In some embodiments, the T-Mixer has a diameter of about 8 mm. In some embodiments, the T-Mixer has a diameter of about 9 mm. In some embodiments, the T-Mixer has a diameter of about 10 mm.

### LNP Solution and Flow Rate

In some embodiments, the LNP solution is substantially free of any nucleic acid (e.g., RNA).

In some embodiments, the LNP solution is free of any nucleic acid (e.g., RNA).

In some embodiments, the LNP solution exits the LNP outlet of the T-Mixer at a flow rate of about 500 mL/min or greater, about 550 mL/min or greater, about 600 ml/min or greater, about 650 mL/min or greater, about 700 mL/min or greater, about 750 mL/min or greater, about 800 mL/min or greater, about 850 mL/min or greater, about 900 mL/min or greater, about 950 mL/min or greater, about 1000 mL/min or greater, about 1100 mL/min or greater, about 1200 mL/min or greater, about 1300 mL/min or greater, about 1400 mL/min or greater, about 1500 mL/min or greater, about 2000 mL/min or greater, about 2500 mL/min or greater, about 3000 mL/min or greater, about 3500 mL/min or greater, about 4000 mL/min or greater, about 4500 mL/min or greater, about 950 mL/min or greater, or about 5000 mL/min or greater.

In some embodiments, the LNP solution exits the LNP outlet of the T-Mixer at a flow rate of about 5000 mL/min or less, about 4500 mL/min or less, about 4000 mL/min or less, about 3500 mL/min or less, about 3000 mL/min or less, about 2500 mL/min or less, about 2000 mL/min or less, about 1500 mL/min or less, about 1400 mL/min or less, about 1300 mL/min or less, about 1200 mL/min or less, about 1100 mL/min or less, about 1000 mL/min or less, about 950 mL/min or less, about 900 mL/min or less, about 850 mL/min or less, about 800 mL/min or less, about 750 mL/min or less, about 700 mL/min or less, about 650 mL/min or less, about 600 mL/min or less, or about 550 mL/min or less.

Combinations of the above-recited ranges for the flow rate are also contemplated (e.g., about 500 mL/min to about 900 mL/min, 500 mL/min to about 950 mL/min, 500 mL/min to about 1000 mL/min, 550 mL/min to about 1000 mL/min, 600 mL/min to about 1000 mL/min, or 650 mL/min to about 1000 mL/min.)

In some embodiments, the LNP solution exits the LNP outlet of the T-Mixer at a flow rate of about 500 mL/min, about 520 mL/min, about 540 mL/min, about 560 mL/min, about 580 mL/min, about 600 mL/min, about 620 mL/min, about 640 mL/min, about 660 mL/min, about 680 mL/min, about 700 mL/min, about 720 mL/min, about 740 mL/min, about 760 mL/min, about 780 mL/min, about 800 mL/min, about 820 mL/min, about 840 mL/min, about 860 mL/min, about 880 mL/min, about 900 mL/min, about 920 mL/min, about 940 mL/min, about 960 mL/min, about 980 mL/min, about 1000 mL/min, about 1100 mL/min, about 1200 mL/min, about 1300 mL/min, about 1400 mL/min, about 1500 mL/min, about 2000 mL/min, about 2500 mL/min, about 3000 mL/min, about 3500 mL/min, about 4000 mL/min, about 4500 mL/min, or about 5000 mL/min.

### LNPs

In some embodiments, the LNPs are substantially free of any nucleic acid (e.g., RNA).

In some embodiments, the LNPs are free of any nucleic acid (e.g., RNA).

In some embodiments, the LNPs have an average diameter of about 100 nm or less, about 95 nm or less, about 90 nm or less, about 85 nm or less, about 80 nm or less, about 75 nm or less, about 70 nm or less, about 65 nm or less, about 60 nm or less, about 55 nm or less, about 50 nm or less, about 45 nm or less, about 40 nm or less, about 35 nm or less, about 30 nm or less, about 25 nm or less, about 20 nm or less, or about 15 nm or less.

In some embodiments, the LNPs have an average diameter of about 10 nm or greater, about 15 nm or greater, about 20 nm or greater, about 25 nm or greater, about 30 nm or greater, about 35 nm or greater, about 40 nm or greater, about 45 nm or greater, about 50 nm or greater, about 55 nm or greater, about 65 nm or greater, about 70 nm or greater, about 75 nm or greater, about 80 nm or greater, about 85 nm or greater, about 90 nm or greater, or about 95 nm or greater.

Combinations of the above-recited ranges for the LNPs average diameter are also contemplated (e.g., about 10 nm to about 90 nm, about 10 nm to about 95 nm, about 10 nm to about 100 nm, about 15 nm to about 100 nm, about 20 nm to about 100 nm, or about 25 nm to about 100 nm.)

In some embodiments, the LNPs have an average diameter of about 10±5 nm, about 15±5 nm, about 20±5 nm, about 25±5 nm, about 30±5 nm, about 35±5 nm, about 40±5 nm, about 45±5 nm, about 50±5 nm, about 55±5 nm, about 60±5 nm, about 65±5 nm, about 70±5 nm, about 75±5 nm, about 80±5 nm, about 85±5 nm, about 90±5 nm, about 95±5 nm, or about 100±5 nm.

### LNPs and LNP Solutions

In some aspects, the present disclosure provides a lipid nanoparticle solution (LNP solution) being prepared by a process described herein.

In some aspects, the present disclosure provides a lipid nanoparticle (LNP) being prepared by a process described herein.

### Ionizable Lipids

The present disclosure provides ionizable lipids. In some embodiments, the ionizable lipids include a central amine moiety and at least one biodegradable group. In some embodiments, the ionizable lipid is an amino lipid. The lipids described herein may be advantageously used in lipid nanoparticles and lipid nanoparticle formulations for the delivery of therapeutic and/or prophylactics, such as a nucleic acid, to mammalian cells or organs.

In some aspects, the ionizable lipids of the present disclosure may be one or more of compounds of Formula (IL-1):

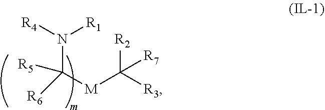

or their N-oxides, or salts or isomers thereof, wherein:
R¹ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
each R⁵ is independently selected from the group consisting of OH, C₁₋₃ alkyl, C₂₋₃ alkenyl, and H;
each R⁶ is independently selected from the group consisting of OH, C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R⁷ is selected from the group consisting of C₁₋₃ alkyl, C₂₋₃ alkenyl, and H;
R⁸ is selected from the group consisting of C₃₋₆ carbocycle and heterocycle;
R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
R¹⁰ is selected from the group consisting of H, OH, C₁₋₃ alkyl, and C₂₋₃ alkenyl;
each R is independently selected from the group consisting of C₁₋₃ alkyl, C₂₋₃ alkenyl, (CH₂)_{q}OR*, and H,
and each q is independently selected from 1, 2, and 3;
each R′ is independently selected from the group consisting of C₁₋₁₈ alkyl, C₂₋₁₈ is alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some aspects, the ionizable lipids of the present disclosure may be one or more of compounds of Formula (IL-X):

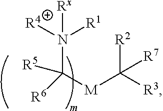

### (IL-X) or its N-oxide,

or a salt or isomer thereof, wherein
   or a salt or isomer thereof, wherein
   R¹ is selected from the group consisting of C₅₋₃₀ alkyl, C₅₋₂₀ alkenyl, —R*YR″, —YR″, and —R″M′R′;
   R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
   R⁴ is selected from the group consisting of hydrogen, a C₃₋₆ carbocycle, —(CH₂)ₙQ, —(CH₂)ₙCHQR, —(CH₂)ₒC(R¹⁰)₂(CH₂)ₙ₋ₒQ,
   —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, N(R)R⁸, —N(R)S(O)₂R⁸, —O(CH₂)ₙOR, —N(R)C(═NR⁹)N(R)₂, —N(R)C(═CHR⁹)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR⁹)N(R)₂, —N(OR)C(═CHR⁹)N(R)₂, —C(═NR⁹)N(R)₂, —C(═NR⁹)R, —C(O)N(R)OR, and —C(R)N(R)₂C(O)OR, each o is independently selected from 1, 2, 3, and 4, and each n is independently selected from 1, 2, 3, 4, and 5;
   R^{x} is selected from the group consisting of C₁₋₆ alkyl, C₂₋₆ alkenyl, —(CH₂)ᵥOH, and —(CH₂)ᵥN(R)₂,
   wherein v is selected from 1, 2, 3, 4, 5, and 6;
   each R⁵ is independently selected from the group consisting of OH, C₁₋₃ alkyl, C₂₋₃ alkenyl, and H;
   each R⁶ is independently selected from the group consisting of OH, C₁₋₃ alkyl, C₂₋₃ alkenyl, and H;
   M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂-13 alkenyl;
   R⁷ is selected from the group consisting of C₁₋₃ alkyl, C₂₋₃ alkenyl, and H;
   R⁸ is selected from the group consisting of C₃₋₆ carbocycle and heterocycle;
   R⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
   R¹⁰ is selected from the group consisting of H, OH, C₁₋₃ alkyl, and C₂₋₃ alkenyl;
   each R is independently selected from the group consisting of C₁₋₃ alkyl, C₂₋₃ alkenyl, (CH₂)_{q}OR*, and H,
and each q is independently selected from 1, 2, and 3;
   each R′ is independently selected from the group consisting of C₁₋₁₈ alkyl, C₂₋₁₈ alkenyl, —R*YR″, —YR″, and H;
   each R″ is independently selected from the group consisting of C₃₋₁₅ alkyl and C₃₋₁₅ alkenyl;
   each R* is independently selected from the group consisting of C₁₋₁₂ alkyl and C₂₋₁₂ alkenyl;
   each Y is independently a C₃₋₆ carbocycle;
   each X is independently selected from the group consisting of F, Cl, Br, and I; and
   m is selected from 5, 6, 7, 8, 9, 10, 11, 12, and 13.

In some embodiments, a subset of compounds of Formula (IL-I) includes those of Formula (IL-IA):

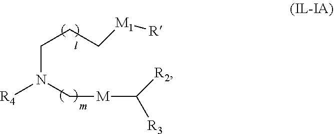

or its N-oxide, or a salt or isomer thereof, wherein l is selected from 1, 2, 3, 4, and 5; m is selected from 5, 6, 7, 8, and 9; M₁ is a bond or M′; R⁴ is hydrogen, unsubstituted C₁₋₃ alkyl, —(CH₂)ₒC(R¹⁰)₂(CH₂)ₙ₋ₒQ, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R² and R³ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In some embodiments, a subset of compounds of Formula (I) includes those of Formula (IL-IB):

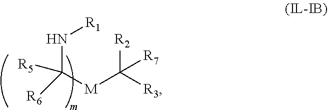

or its N-oxide, or a salt or isomer thereof, in which all variables are as defined herein. In some embodiments, m is selected from 5, 6, 7, 8, and 9; R₄ is hydrogen, unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl, M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl. In some embodiments, m is 5, 7, or 9. In some embodiments, Q is OH, —NHC(S)N(R)₂, or —NHC(O)N(R)₂. In some embodiments, Q is —N(R)C(O)R, or —N(R)S(O)₂R.

In some embodiments, a subset of compounds of Formula (IL-I) includes those of Formula (IL-II):

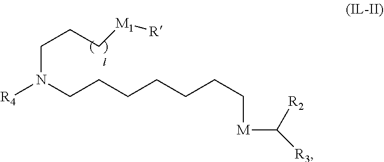

or its N-oxide, or a slat or isomer thereof, wherein l is selected from 1, 2, 3, 4 and 5; M1 is a bond or M′; R₄ is hydrogen, unsubstituted C₁₋₃ alkyl, or —(CH₂)ₙQ, in which n is 2, 3, or 4, and Q is —OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl.

In some aspects, the ionizable lipids of the present disclosure may be one or more of compounds of Formula (IL-VI):

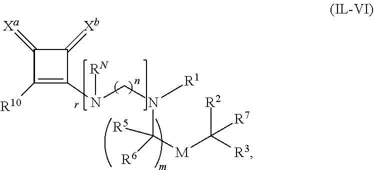

### or its N-oxide,

or a salt or isomer thereof, wherein
   R¹ is selected from the group consisting of C₅₋₃₀ alkyl, C₅₋₂₀ alkenyl, —R*YR″, —YR″, and —R″M′R′;
   R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
   each R⁵ is independently selected from the group consisting of OH, C₁₋₃ alkyl, C₂₋₃ alkenyl, and H;
   each R^{b} is independently selected from the group consisting of OH, C₁₋₃ alkyl, C₂₋₃ alkenyl, and H;
   M and M′ are independently selected from —C(O)O—, —OC(O)—, —OC(O)-M″—C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
   R⁷ is selected from the group consisting of C₁₋₃ alkyl, C₂₋₃ alkenyl, and H;
   each R is independently selected from the group consisting of H, C₁₋₃ alkyl, and C₂₋₃ alkenyl;
   R^{N} is H, or C₁₋₃ alkyl;
   each R′ is independently selected from the group consisting of C₁₋₁₈ alkyl, C₂₋₁₈ alkenyl, —R*YR″, —YR″, and H;
   each R″ is independently selected from the group consisting of C₃₋₁₈ alkyl and C₃₋₁₅ alkenyl;
   each R* is independently selected from the group consisting of C₁₋₁₂ alkyl and C₂₋₁₂ alkenyl;
   each Y is independently a C₃₋₆ carbocycle;
   each X is independently selected from the group consisting of F, Cl, Br, and I;
   X^{a} and X^{b} are each independently O or S;
   R¹⁰ is selected from the group consisting of H, halo, —OH, R, —N(R)₂, —CN, —N₃, —C(O)OH, —C(O)OR, —OC(O)R, —OR, —SR, —S(O)R, —S(O)OR, —S(O)₂OR, —NO₂, —S(O)₂N(R)₂, —N(R)S(O)₂R, —NH(CH₂)ₜ₁N(R)₂, —NH(CH₂)ₚ₁O(CH₂)_{q1}N(R)₂, —NH(CH₂)ₛ₁OR, —N((CH₂)ₛ₁OR)₂, a carbocycle, a heterocycle, aryl and heteroaryl;
   m is selected from 5, 6, 7, 8, 9, 10, 11, 12, and 13;
   n is selected from 1, 2, 3, 4, 5, 6, 7, 8, 9, and 10;
   r is 0 or 1;
   t¹ is selected from 1, 2, 3, 4, and 5;
   p¹ is selected from 1, 2, 3, 4, and 5;
   q¹ is selected from 1, 2, 3, 4, and 5; and
   s¹ is selected from 1, 2, 3, 4, and 5.

In some embodiments, a subset of compounds of Formula (IL-VI) includes those of Formula (IL-VI-a):

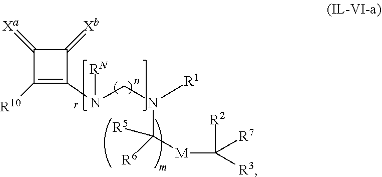

or its N-oxide, or a salt or isomer thereof, wherein
   R^{1a} and R^{1b} are independently selected from the group consisting of C₁₋₁₄ alkyl and C₂₋₁₄ alkenyl; and
   R² and R³ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle.

In another embodiment, a subset of compounds of Formula (IL-VI) includes those of Formula (IL-VII):

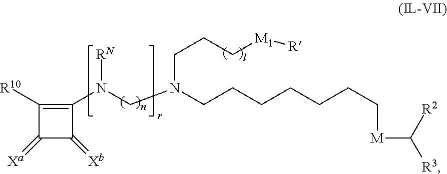

or its N-oxide, or a salt or isomer thereof, wherein
   l is selected from 1, 2, 3, 4, and 5;
   M₁ is a bond or M′; and
   R² and R₃ are independently selected from the group consisting of H, C₁₋₁₄ alkyl, and C₂₋₁₄ alkenyl.

In another embodiment, a subset of compounds of Formula (IL-VI) includes those of Formula (IL-V III):

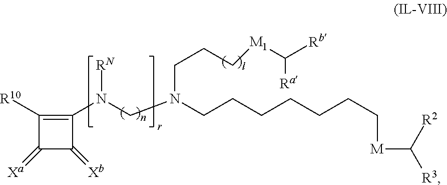

or its N-oxide, or a salt or isomer thereof, wherein
   l is selected from 1, 2, 3, 4, and 5;
   M₁ is a bond or M′; and
   R^{a′} and R^{b′} are independently selected from the group consisting of C₁₋₁₄ alkyl and C₂₋₁₄ alkenyl; and
   R² and R³ are independently selected from the group consisting of C₁₋₁₄ alkyl, and C₂-14 alkenyl.

The compounds of any one of formula (IL-I), (IL-IA), (IL-VI), (IL-VI-a), (IL-VII) or (IL-VIII) include one or more of the following features when applicable

In some embodiments, M₁ is M′.

In some embodiments, M and M′ are independently —C(O)O— or —OC(O)—.

In some embodiments, at least one of M and M′ is —C(O)O— or —OC(O)—.

In some embodiments, at least one of M and M′ is —OC(O)—.

In some embodiments, M is —OC(O)— and M′ is —C(O)O—. In some embodiments, M is —C(O)O— and M′ is —OC(O)—. In some embodiments, M and M′ are each —OC(O)—. In some embodiments, M and M′ are each —C(O)O—.

In some embodiments, at least one of M and M′ is —OC(O)-M″—C(O)O—.

In some embodiments, M and M′ are independently —S—S—.

In some embodiments, at least one of M and M′ is —S—S—.

In some embodiments, one of M and M′ is —C(O)O— or —OC(O)— and the other is —S—S—. For example, M is —C(O)O— or —OC(O)— and M′ is —S—S— or M′ is —C(O)O—, or —OC(O)— and M is —S—S—.

In some embodiments, one of M and M′ is —OC(O)-M″—C(O)O—, in which N″ is a bond, C₁₋₁₃ alkyl or C₂₋₃ alkenyl. In other embodiments, M″ is C₁₋₆ alkyl or C₂₋₆ alkenyl.

In some embodiments, M″ is C₁₋₄ alkyl or C₂₋₄ alkenyl. For example, in some embodiments, M″ is C₁ alkyl. For example, in some embodiments, M″ is C₂ alkyl. For example, in some embodiments, M″ is C₃ alkyl. For example, in some embodiments, M″ is C₄ alkyl. For example, in some embodiments, M″ is C₂ alkenyl. For example, in some embodiments, M″ is C₃ alkenyl. For example, in some embodiments, M″ is C₄ alkenyl.

In some embodiments, l is 1, 3, or 5.

In some embodiments, R⁴ is hydrogen.

In some embodiments, R⁴ is not hydrogen.

In some embodiments, R⁴ is unsubstituted methyl or —(CH₂)ₙQ, in which Q is OH, —NHC(S)N(R)₂, —NHC(O)N(R)₂, —N(R)C(O)R, or —N(R)S(O)₂R.

In some embodiments, Q is OH.

In some embodiments, Q is —NHC(S)N(R)₂.

In some embodiments, Q is —NHC(O)N(R)₂.

In some embodiments, Q is —N(R)C(O)R.

In some embodiments, Q is —N(R)S(O)₂R.

In some embodiments, Q is —O(CH₂)ₙN(R)₂.

In some embodiments, Q is —O(CH₂)ₙOR.

In some embodiments, Q is —N(R)R⁸.

In some embodiments, Q is —NHC(═NR⁹)N(R)₂.

In some embodiments, Q is —NHC(═CHR⁹)N(R)₂.

In some embodiments, Q is —OC(O)N(R)₂.

In some embodiments, Q is —N(R)C(O)OR.

In some embodiments, n is 2.

In some embodiments, n is 3.

In some embodiments, n is 4.

In some embodiments, M₁ is absent.

In some embodiments, at least one R⁵ is hydroxyl. For example, one R⁵ is hydroxyl.

In some embodiments, at least one R⁶ is hydroxyl. For example, one R⁵ is hydroxyl.

In some embodiments one of R⁵ and R⁶ is hydroxyl. For example, one R⁵ is hydroxyl and each R⁶ is hydrogen. For example, one R⁶ is hydroxyl and each R⁵ is hydrogen.

In some embodiments, R^{x} is C₁₋₆ alkyl. In some embodiments, R is C₁₋₃ alkyl. For example, R^{x} is methyl. For example, R^{x} is ethyl. For example, R^{x} is propyl.

In some embodiments, R^{x} is —(CH₂)ᵥOH and, v is 1, 2 or 3. For example, R^{x} is methanoyl. For example, R^{x} is ethanoyl. For example, R^{x} is propanoyl.

In some embodiments, R^{x} is —(CH₂)˜N(R)₂, v is 1, 2 or 3 and each R is H or methyl. For example, R^{x} is methanamino, methylmethanamino, or dimethylmethanamino. For example, R″ is aminomethanyl, methylaminomethanyl, or dimethylaminomethanyl. For example, R″ is aminoethanyl, methylaminoethanyl, or dimethylaminoethanyl. For example, R″ is aminopropanyl, methylaminopropanyl, or dimethylaminopropanyl.

In some embodiments, R′ is C₁₋₁₈ alkyl, C₂₋₁₈ alkenyl, —R*YR″, or —YR″.

In some embodiments, R² and R³ are independently C₃₋₁₄ alkyl or C₃₋₁₄ alkenyl.

In some embodiments, R^{1b} is C₁₋₁₄ alkyl. In some embodiments, R^{1b} is C₂₋₁₄ alkyl. In some embodiments, R^{1b} is C₃₋₁₄ alkyl. In some embodiments, R^{1b} is C₁₋₈ alkyl. In some embodiments, R^{1b} is C₁₋₅ alkyl. In some embodiments, R^{1b} is C₁₋₃ alkyl. In some embodiments, R^{1b} is selected from C₁ alkyl, C₂ alkyl, C₃ alkyl, C₄ alkyl, and C₅ alkyl. For example, in some embodiments, R^{1b} is C₁ alkyl. For example, in some embodiments, R^{1b} is C₂ alkyl. For example, in some embodiments, R^{1b} is C₃ alkyl. For example, in some embodiments, R^{1b} is C₄ alkyl. For example, in some embodiments, R^{1b} is C₅ alkyl.

In some embodiments, R⁷ is different from —(CHR⁵R⁶)ₘ-M-CR²R₃R⁷.

In some embodiments, —CHR^{1a}R^{1b}— is different from —(CHR⁵R⁶)ₘ-M-CR²R³R⁷.

In some embodiments, R⁷ is H. In some embodiments, R⁷ is selected from C₁₋₃ alkyl. For example, in some embodiments, R⁷ is C₁ alkyl. For example, in some embodiments, R⁷ is C₂ alkyl. For example, in some embodiments, R⁷ is C₃ alkyl. In some embodiments, R⁷ is selected from C₄ alkyl, C₄ alkenyl, C₅ alkyl, C₅ alkenyl, C₆ alkyl, C₆ alkenyl, C₇ alkyl, C₇ alkenyl, C₉ alkyl, C₉ alkenyl, C₁₁ alkyl, C₁₁ alkenyl, C₁₇ alkyl, C₁₇ alkenyl, C₁₈ alkyl, and C₁₈ alkenyl.

In some embodiments, Rb′ is C₁₋₁₄ alkyl. In some embodiments, Rb′ is C₂-14 alkyl. In some embodiments, R^{b′} is C₃₋₁₄ alkyl. In some embodiments, R^{b′} is C₁₋₈ alkyl. In some embodiments, R^{b′} is C₁₋₅ alkyl. In some embodiments, R^{b′} is C₁₋₃ alkyl. In some embodiments, R^{b′} is selected from C₁ alkyl, C₂ alkyl, C₃ alkyl, C₄ alkyl and C₅ alkyl. For example, in some embodiments, R^{b′} is C₁ alkyl. For example, in some embodiments, R^{b′} is C₂ alkyl. For example, some embodiments, R^{b′} is C₃ alkyl. For example, some embodiments, R^{b′} is C₄ alkyl.

In one embodiment, the compounds of Formula (IL-I) are of Formula (IL-IIa):

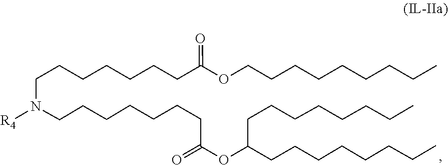

or their N-oxides, or salts or isomers thereof, wherein R₄ is as described herein.

In another embodiment, the compounds of Formula (IL-I) are of Formula (IL-Ib):

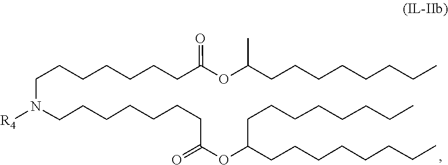

or their N-oxides, or salts or isomers thereof, wherein R₄ is as described herein.

In another embodiment, the compounds of Formula (IL-I) are of Formula (IL-IIc) or (IL-IIe):

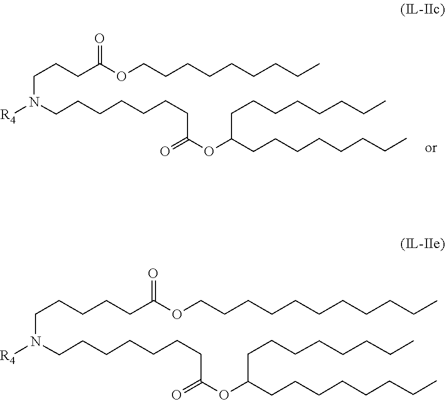

or their N-oxides, or salts or isomers thereof, wherein R₄ is as described herein.

In another embodiment, the compounds of Formula (IL-I) are of Formula (IL-IIf):

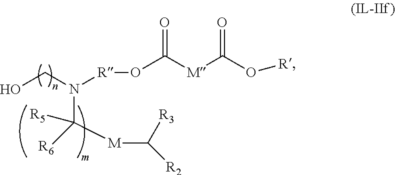

or their N-oxides, or salts or isomers thereof, wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (IL-I) are of Formula (IL-IId):

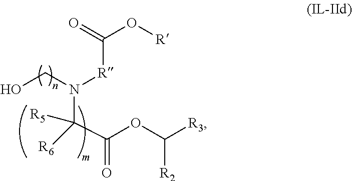

or their N-oxides, or salts or isomers thereof, wherein n is 2, 3, or 4; m, R′, R″, and R₂ through R₆ are as described herein. In some embodiments, each of R₂ and R₃ may be independently selected from the group consisting of C₅₋₁₄ alky and C₅₋₁₄ alkenyl.

In a further embodiment, the compounds of Formula (IL-I) are of Formula (IL-IIg):

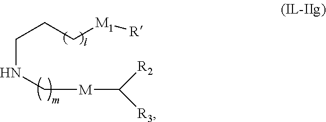

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. In some embodiments, M″ is C₁₋₆ alkyl (e.g., C₁₋₄ alkyl) or C₂₋₆ alkenyl (e.g., C₂₋₄ alkenyl). In some embodiments, R₂ and R₃ are independently selected from the group consisting of C₅₋₄ alkyl and C₅₋₁₄ alkenyl.

In another embodiment, a subset of compounds of Formula (IL-VI) includes those of Formula (IL-VIIa):

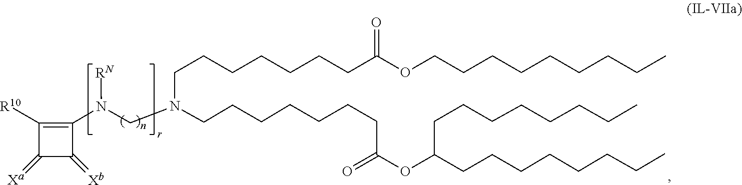

or its N-oxide, or a salt or isomer thereof.

In another embodiment, a subset of compounds of Formula (VI) includes those of Formula (IL-VIIIa):

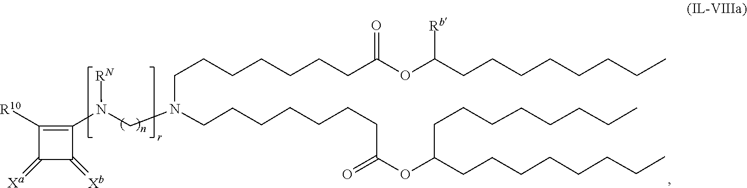

or its N-oxide, or a salt or isomer thereof.

In another embodiment, a subset of compounds of Formula (IL-VI) includes those of Formula (IL-VIIIb):

or its N-oxide, or a salt or isomer thereof.

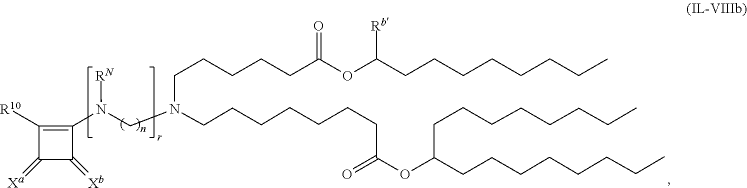

In another embodiment, a subset of compounds of Formula (IL-VI) includes those of Formula (IL-VIIb-1);

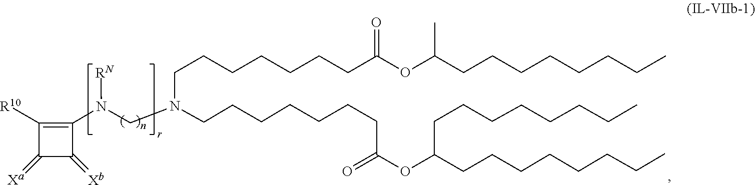

or its N-oxide, or a salt or isomer thereof.

In another embodiment, a subset of compounds of Formula (IL-VI) includes those of Formula (IL-VIIb-2):

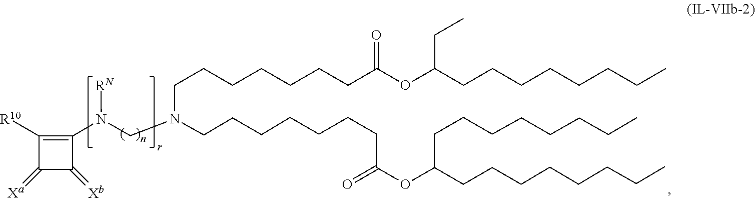

or its N-oxide, or a salt or isomer thereof.

In another embodiment, a subset of compounds of Formula (IL-VI) includes those of Formula (IL-VIIb-3):

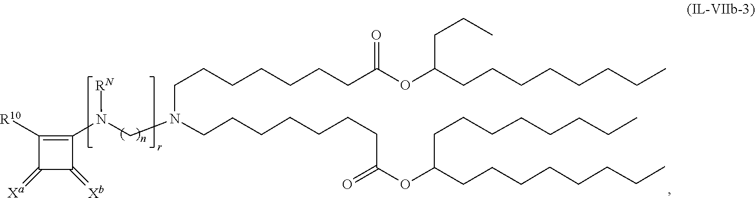

or its N-oxide, or a salt or isomer thereof.

In another embodiment, a subset of compounds of Formula (IL-VI) includes those of Formula (IL-VIIc):

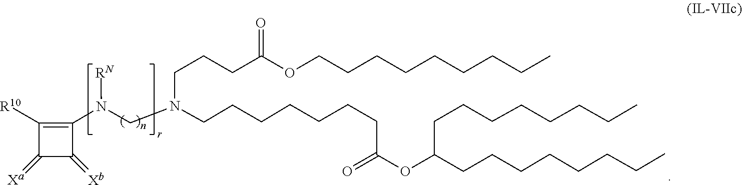

In another embodiment, a subset of compounds of Formula (IL-VI) includes those of Formula (IL-VIId):

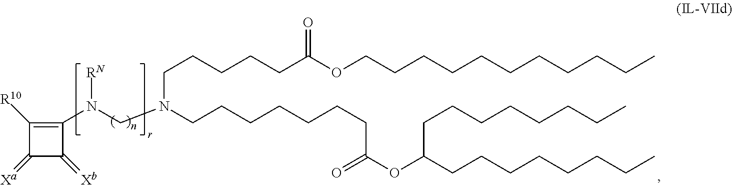

or its N-oxide, or a salt or isomer thereof.

In another embodiment, a subset of compounds of Formula (IL-VI) includes those of Formula (IL-VIIIc):

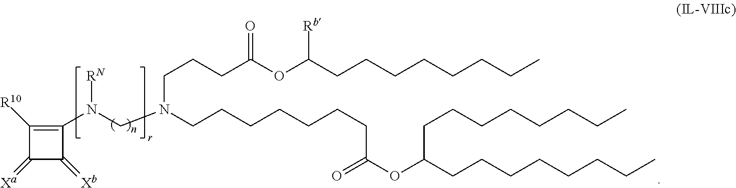

In another embodiment, a subset of compounds of Formula (IL-VI) includes those of Formula (IL-VIIId):

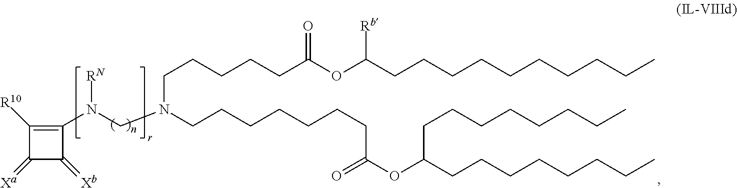

or its N-oxide, or a salt or isomer thereof.

The compounds of any one of formulae (IL-I), (IL-IA), (IL-IB), (IL-II), (IL-IIa), (IL-IIb), (IL-IIc), (IL-IId), (IL-IIe), (IL-IIf), (IL-IIg), (IL-III), (IL-VI), (IL-VI-a), (IL-VII), (IL-VIII), (IL-VIIa), (IL-VIIIa), (IL-VIIIb), (IL-VIIb-1), (IL-VIIb-2), (IL-VIIb-3), (IL-VIIc), (IL-VIId), (IL-VIIIc), or (IL-VIIId) include one or more of the following features when applicable.

In some embodiments, the ionizable lipids are one or more of the compounds described in PCT Application Nos. PCT/US2020/051613, PCT/US2020/051613, and PCT/US2020/051629, and in PCT Publication Nos. WO 2017/049245, WO 2018/170306, WO 2018/170336, WO 2020/061367.

In some embodiments, the ionizable lipids are selected from Compounds 1-280 described in U.S. Application No. 62/475,166.

In some embodiments, the ionizable lipid is

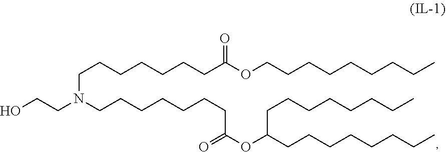 [SMILES: CCCCCCCCCOC(=O)CCCCCCCN(CCO)CCCCCCCC(=O)OC(CCCCCCCC)CCCCCCCC]

or a salt thereof.

In some embodiments, the ionizable lipid is (IL-1).

In some embodiments, the ionizable lipid is

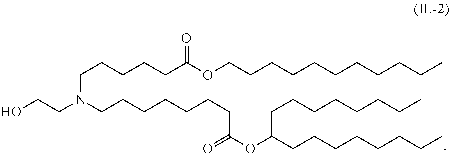 [SMILES: CCCCCCCCCCCOC(=O)CCCCCN(CCO)CCCCCCCC(=O)OC(CCCCCCCC)CCCCCCCC]

or a salt thereof.

In some embodiments, the ionizable lipid is IL-2.

In some embodiments, the ionizable lipid is

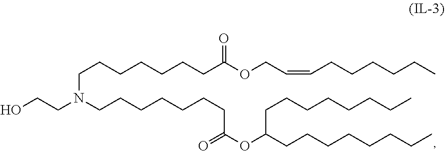 [SMILES: CCCCCC/C=C\COC(=O)CCCCCCCN(CCO)CCCCCCCC(=O)OC(CCCCCCCC)CCCCCCCC]

or a salt thereof.

In some embodiments, the ionizable lipid is IL-3.

In some embodiments, the ionizable lipid is

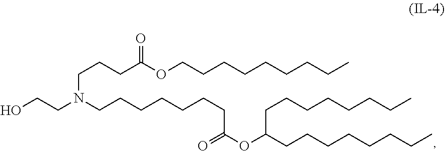 [SMILES: CCCCCCCCCOC(=O)CCCN(CCO)CCCCCCCC(=O)OC(CCCCCCCC)CCCCCCCC]

or a salt thereof.

In some embodiments, the ionizable lipid is IL-4.

In some embodiments, the ionizable lipid is

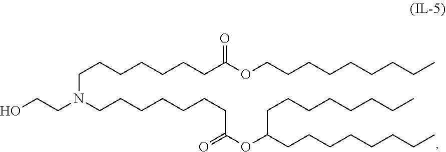 [SMILES: CCCCCCCCCOC(=O)CCCCCCCN(CCO)CCCCCCCC(=O)OC(CCCCCCCC)CCCCCCCC]

or a salt thereof.

In some embodiments, the ionizable lipid is IL-5.

In some embodiments, the ionizable lipid is

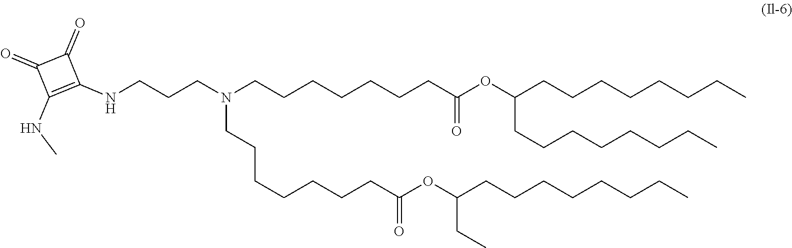 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or a salt thereof.

In some embodiments, the ionizable lipid is IL-6.

In some embodiments, the ionizable lipid is

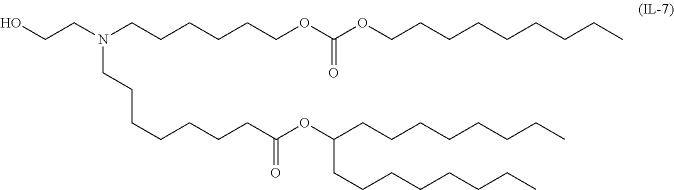 [SMILES: CCCCCCCCCOC(=O)OCCCCCCN(CCO)CCCCCCCC(=O)OC(CCCCCCCC)CCCCCCCC]

or a salt thereof.

In some embodiments, the ionizable lipid is IL-7.

In some embodiments, the ionizable lipid is

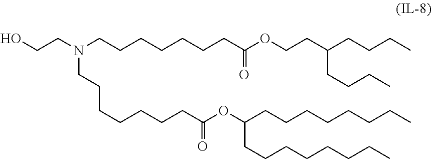 [SMILES: CCCCCCCCC(CCCCCCCC)OC(=O)CCCCCCCN(CCO)CCCCCCCC(=O)OCCC(CCCC)CCCC]

or a salt thereof.

In some embodiments, the ionizable lipid is IL-8.

In some embodiments, the ionizable lipid is

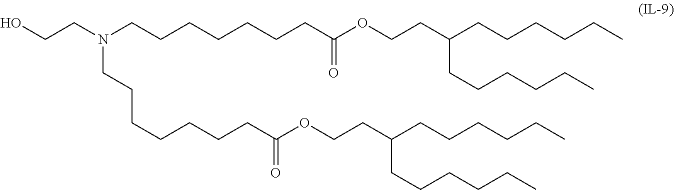 [SMILES: CCCCCCC(CCCCCC)CCOC(=O)CCCCCCCN(CCO)CCCCCCCC(=O)OCCC(CCCCCC)CCCCCC]

or a salt thereof.

In some embodiments, the ionizable lipid is IL-9.

In some embodiments, the ionizable lipid is

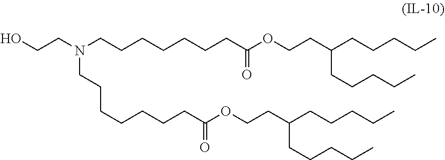 [SMILES: CCCCCC(CCCCC)CCOC(=O)CCCCCCCN(CCO)CCCCCCCC(=O)OCCC(CCCCC)CCCCC]

or a salt thereof.

In some embodiments, the ionizable lipid is IL-10.

In some embodiments, the ionizable lipid is 11)

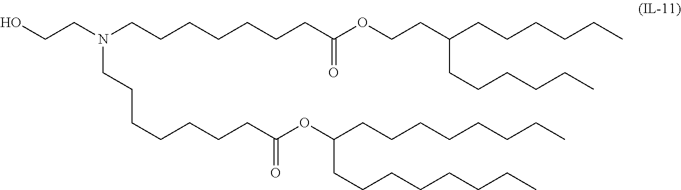 [SMILES: CCCCCCCCC(CCCCCCCC)OC(=O)CCCCCCCN(CCO)CCCCCCCC(=O)OCCC(CCCCCC)CCCCCC]

or a salt thereof.

In some embodiments, the ionizable lipid is IL-11.

In some embodiments, the ionizable lipid is

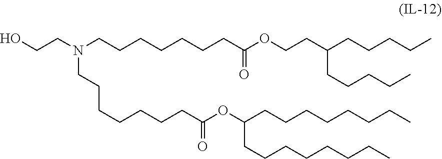 [SMILES: CCCCCCCCC(CCCCCCCC)OC(=O)CCCCCCCN(CCO)CCCCCCCC(=O)OCCC(CCCCC)CCCCC]

or a salt thereof.

In some embodiments, the ionizable lipid is IL-12.

In some embodiments, the ionizable lipid is

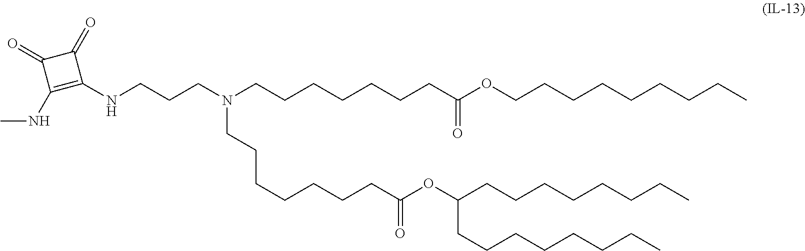 [SMILES: CCCCCCCCCOC(=O)CCCCCCCN(CCCCCCCC(=O)OC(CCCCCCCC)CCCCCCCC)CCCNc1c(NC)c(=O)c1=O]

or a salt thereof.

In some embodiments, the ionizable lipid is IL-13.

In some embodiments, the ionizable lipid is

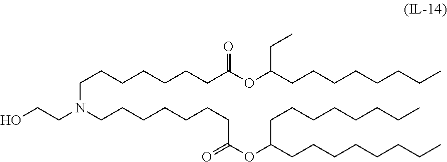 [SMILES: CCCCCCCCC(CC)OC(=O)CCCCCCCN(CCO)CCCCCCCC(=O)OC(CCCCCCCC)CCCCCCCC]

or a salt thereof.

In some embodiments, the ionizable lipid is IL-14.

In some embodiments, the ionizable lipid is

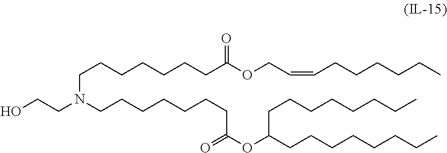 [SMILES: CCCCCC/C=C\COC(=O)CCCCCCCN(CCO)CCCCCCCC(=O)OC(CCCCCCCC)CCCCCCCC]

or a salt thereof.

In some embodiments, the ionizable lipid is IL-15.

In some embodiments, the ionizable lipid is

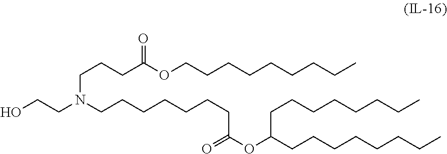 [SMILES: CCCCCCCCCOC(=O)CCCN(CCO)CCCCCCCC(=O)OC(CCCCCCCC)CCCCCCCC]

or a salt thereof.

In some embodiments, the ionizable lipid is IL-16.

In some embodiments, the ionizable lipid is

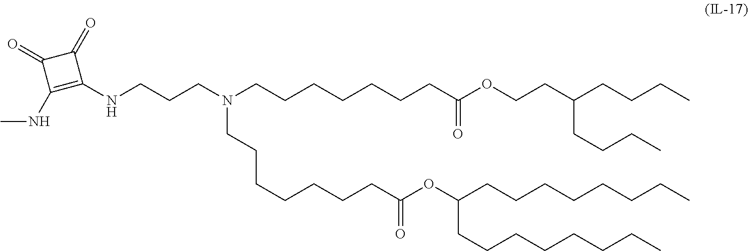 [SMILES: CCCCCCCCC(CCCCCCCC)OC(=O)CCCCCCCN(CCCCCCCC(=O)OCCC(CCCC)CCCC)CCCNc1c(NC)c(=O)c1=O]

or a salt thereof.

In some embodiments, the ionizable lipid is IL-17.

In some embodiments, the ionizable lipid is

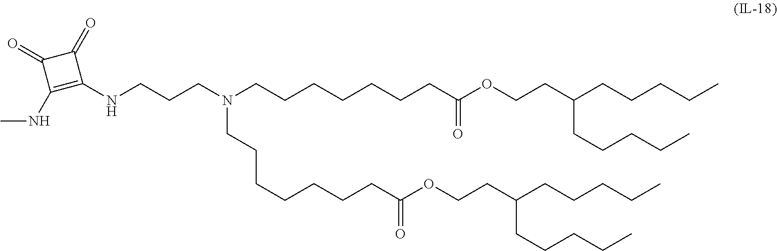 [SMILES: CCCCCC(CCCCC)CCOC(=O)CCCCCCCN(CCCCCCCC(=O)OCCC(CCCCC)CCCCC)CCCNc1c(NC)c(=O)c1=O]

or a salt thereof.

In some embodiments, the ionizable lipid is IL-18.

In some embodiments, the ionizable lipid is

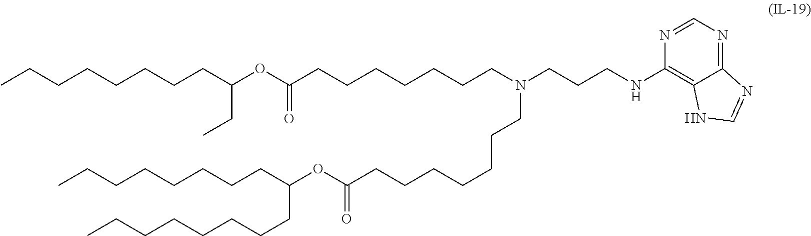 [SMILES: CCCCCCCCC(CC)OC(=O)CCCCCCCN(CCCCCCCC(=O)OC(CCCCCCCC)CCCCCCCC)CCCNc1ncnc2nc[nH]c12]

or a salt thereof.

In some embodiments, the ionizable lipid is IL-19.

In some aspects, the ionizable lipids of the present disclosure may be one or more of compounds of formula IL-VIVa):

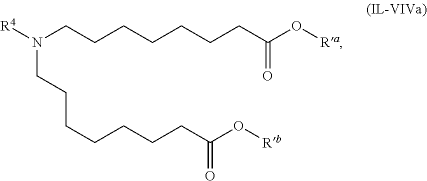

or its N-oxide, or a salt or isomer thereof,
wherein R′^{a} is R′^{branched} or R′^{cyclic}; wherein
R′^{branched} is

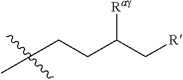

and R′^{cyclic}

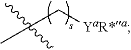

and
R′^{b} is

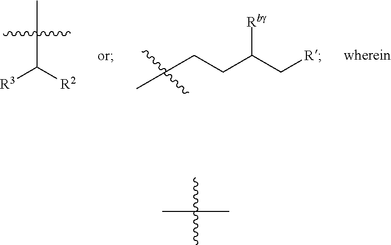

denotes a point of attachment;
   wherein R^{aγ} and R^{bγ} are each independently a C₂₋₁₂ alkyl or C₂₋₁₂ alkenyl;
   R² and R³ are each independently selected from the group consisting of C₁₋₁₄ alkyl and C₂₋₁₄ alkenyl;
   R⁴ is —(CH₂)₂OH;
   each R′ independently is a C₁₋₁₂ alkyl or C₂₋₁₂ alkenyl;
   Y^{a} is a C₃₋₆ carbocycle;
   R*″^{a} is selected from the group consisting of C₁₋₁₅ alkyl and C₂₋₁₅ alkenyl; and
   s is 2 or 3.

In some aspects, the ionizable lipids of the present disclosure may be one or more of compounds of formula (IL-VIVb):

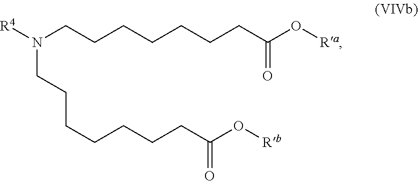

or its N-oxide, or a salt or isomer thereof,
wherein R′^{a} is R′^{branched} or R′^{cyclic}; wherein
R′^{branched} is

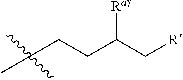

and R′^{cyclic} is

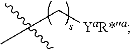

and
R′^{b} is:

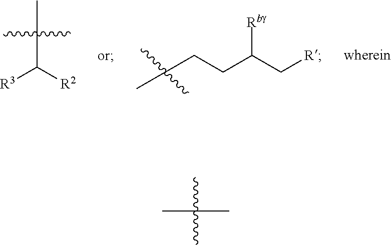

denotes a point of attachment;
   wherein R^{ay} and R^{by} are each independently a C₂₋₁₂ alkyl or C₂₋₁₂ alkenyl;
   R² and R³ are each independently selected from the group consisting of C₁₋₁₄ alkyl and C₂₋₁₄ alkenyl;
   R⁴ is

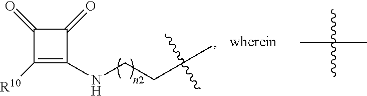

denotes a point of attachment;
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃, carbocycle;
R*″^{a} is selected from the group consisting of C₁₋₁₈ alkyl and C₂₋₁₅ alkenyl; and
s is 2 or 3.

In some embodiments, the ionizable lipid is selected from:

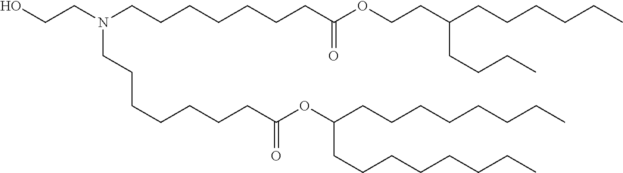 [SMILES: CCCCCCCCC(CCCCCCCC)OC(=O)CCCCCCCN(CCO)CCCCCCCC(=O)OCCC(CCCC)CCCCCC]

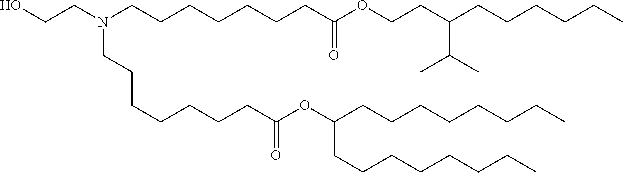 [SMILES: CCCCCCCCC(CCCCCCCC)OC(=O)CCCCCCCN(CCO)CCCCCCCC(=O)OCCC(CCCCCC)C(C)C]

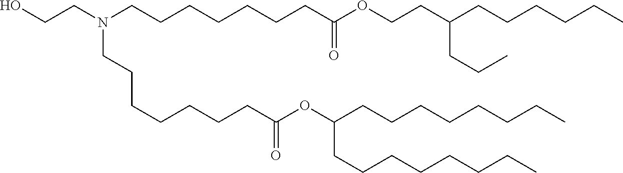 [SMILES: CCCCCCCCC(CCCCCCCC)OC(=O)CCCCCCCN(CCO)CCCCCCCC(=O)OCCC(CCC)CCCCCC]

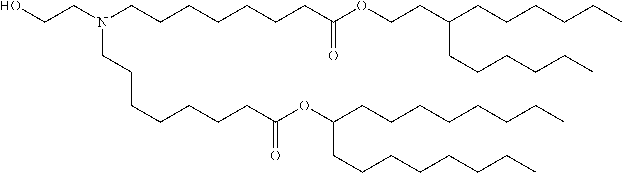 [SMILES: CCCCCCCCC(CCCCCCCC)OC(=O)CCCCCCCN(CCO)CCCCCCCC(=O)OCCC(CCCCCC)CCCCCC]

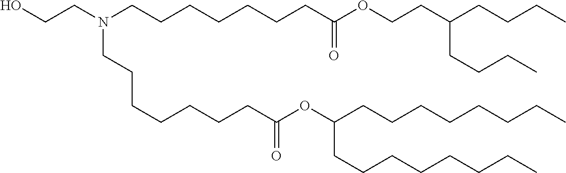 [SMILES: CCCCCCCCC(CCCCCCCC)OC(=O)CCCCCCCN(CCO)CCCCCCCC(=O)OCCC(CCCC)CCCC]

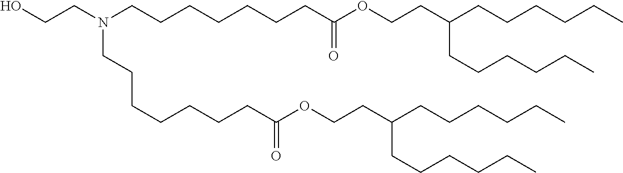 [SMILES: CCCCCCC(CCCCCC)CCOC(=O)CCCCCCCN(CCO)CCCCCCCC(=O)OCCC(CCCCCC)CCCCCC]

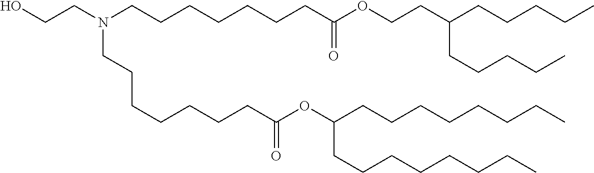 [SMILES: CCCCCCCCC(CCCCCCCC)OC(=O)CCCCCCCN(CCO)CCCCCCCC(=O)OCCC(CCCCC)CCCCC]

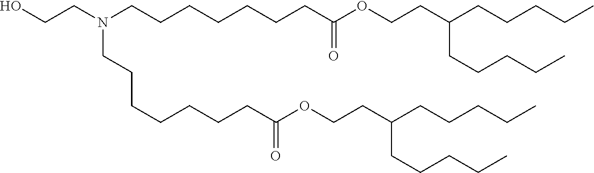 [SMILES: CCCCCC(CCCCC)CCOC(=O)CCCCCCCN(CCO)CCCCCCCC(=O)OCCC(CCCCC)CCCCC]

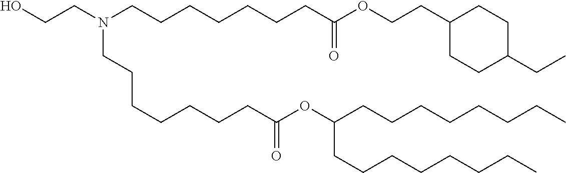 [SMILES: CCCCCCCCC(CCCCCCCC)OC(=O)CCCCCCCN(CCO)CCCCCCCC(=O)OCCC1CCC(CC)CC1]

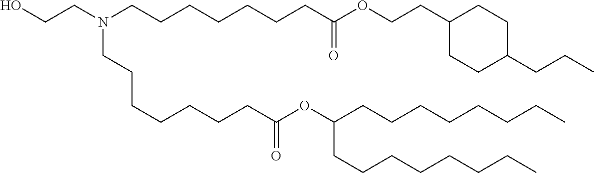 [SMILES: CCCCCCCCC(CCCCCCCC)OC(=O)CCCCCCCN(CCO)CCCCCCCC(=O)OCCC1CCC(CCC)CC1]

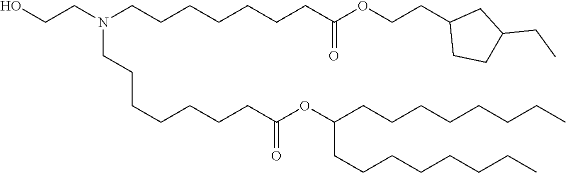 [SMILES: CCCCCCCCC(CCCCCCCC)OC(=O)CCCCCCCN(CCO)CCCCCCCC(=O)OCCC1CCC(CC)C1]

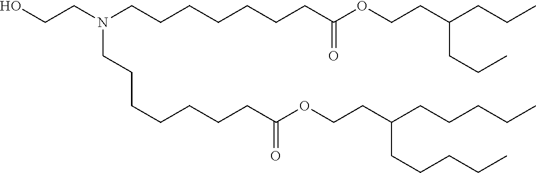 [SMILES: CCCCCC(CCCCC)CCOC(=O)CCCCCCCN(CCO)CCCCCCCC(=O)OCCC(CCC)CCC]

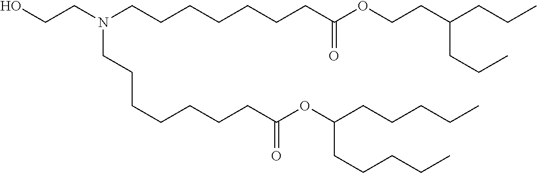 [SMILES: CCCCCC(CCCCC)OC(=O)CCCCCCCN(CCO)CCCCCCCC(=O)OCCC(CCC)CCC]

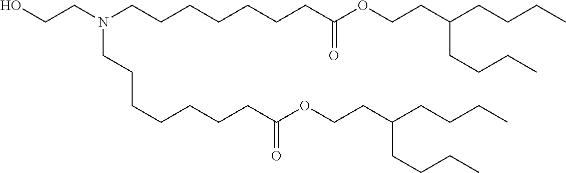 [SMILES: CCCCC(CCCC)CCOC(=O)CCCCCCCN(CCO)CCCCCCCC(=O)OCCC(CCCC)CCCC]

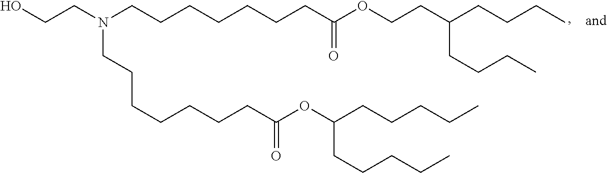 [SMILES: CCCCCC(CCCCC)OC(=O)CCCCCCCN(CCO)CCCCCCCC(=O)OCCC(CCCC)CCCC]

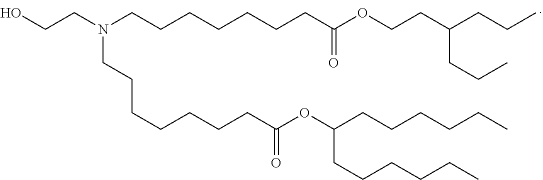 [SMILES: CCCCCCC(CCCCCC)OC(=O)CCCCCCCN(CCO)CCCCCCCC(=O)OCCC(CCC)CCC]

In some aspects, the ionizable lipids of the present disclosure may be one or more of compounds of formula (IL-Ill):

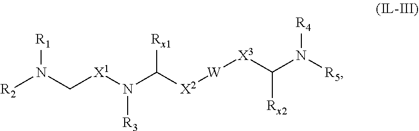

or salts or isomers thereof, wherein,
   W is

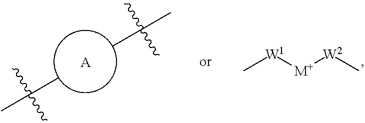

ring A is

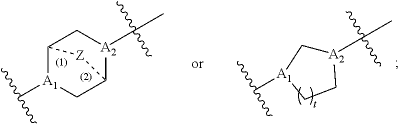

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl:
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₁₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

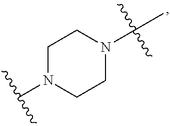 [SMILES: *N1CCN(*)CC1]

then
i) at least one of X¹, X², and X³ is not —CH₂—; and/or
ii) at least one of R₁, R₂, R₃, R₁, and R₅ is —R″MR′.

In some embodiments, the compound is of any of formulae (IL-IIIa1)-(IL-IIIa8):

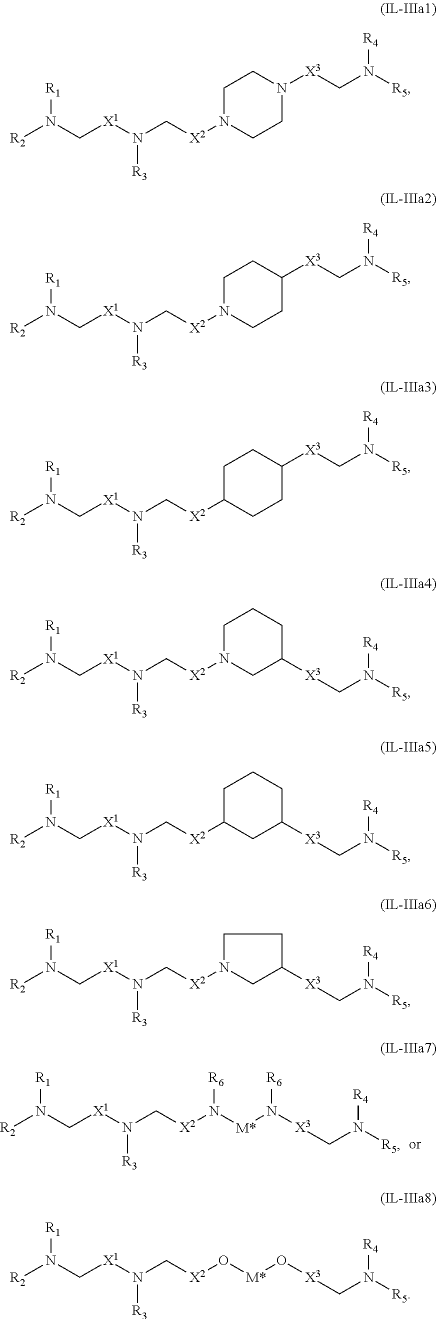

In some embodiments, the ionizable lipids are one or more of the compounds described in PCT Publication Nos. WO 2017/112865, WO 2018/232120.

In some embodiments, the ionizable lipids are selected from Compound 1-156 described in PCT Publication No. WO 2018/232120.

In some embodiments, the ionizable lipids are selected from Compounds 1-16, 42-66, 68-76, and 78-156 described in PCT Publication Nos. WO 2017/112865.

In some embodiments, the ionizable lipid is

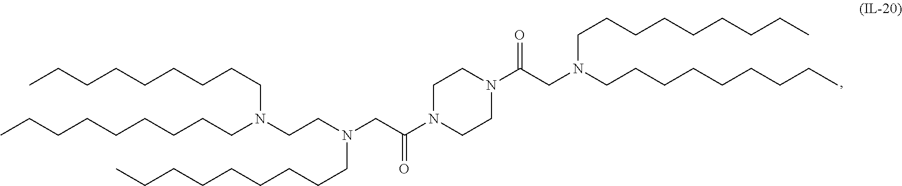 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

In some embodiments, the ionizable lipid is IL-20.

In some embodiments, the ionizable lipid is

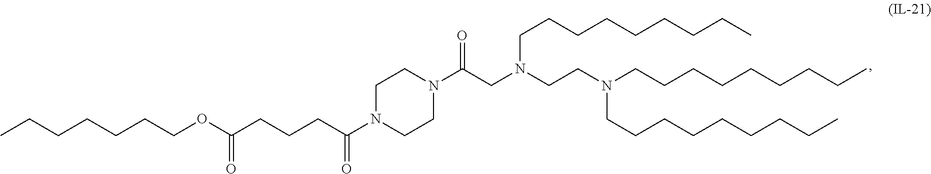 [SMILES: CCCCCCCCCN(CCCCCCCCC)CCN(CCCCCCCCC)CC(=O)N1CCN(C(=O)CCCC(=O)OCCCCCCC)CC1]

or a salt thereof.

In some embodiments, the ionizable lipid is IL-21.

The central amine moiety of a lipid according to Formula (IL-1), (IL-IA), (IL-IB), (IL-II), (IL-IIa), (IL-IIb), (IL-IIc), (IL-IId), (IL-IIe), (IL-IIf), (IL-IIg), (IL-III), (IL-IIIa1), (IL-IIIa2), (IL-IIIa3), (IL-IIIa4), (IL-IIIa5), (IL-IIIa6), (IL-IIIa7), or (IL-IIIa8) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

In some embodiments, the ionizable lipid is selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-I-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

### Polyethylene Glycol (PEG) Lipids

As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid.

In some embodiments, the PEG lipid includes, but are not limited to, 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG lipids includes those having lengths of from about C₁₄ to about C₂₂, In some embodiments, the lipid moiety of the PEG lipids includes those having lengths of from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG lipids are known in the art, such as those described in U.S. Pat. No. 8,158,601 and International Publ. No. WO 2015/130584 A2, which are incorporated herein by reference in their entireties.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle or lipid nanoparticle formulation may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid.

In some embodiments, the PEG-modified lipids are a modified form of PEG DMG. PEG-DMG has the following structure:

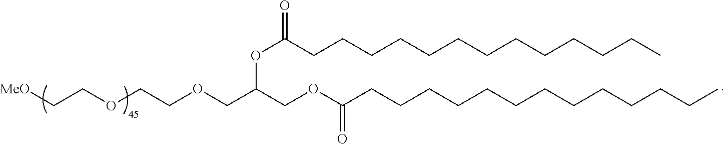

In one embodiment, PEG lipids useful in the present invention can be PEGylated lipids described in International Publication No. WO2012099755, which is herein incorporated by reference in its entirety. Any of these exemplary PEG lipids described herein may be modified to comprise a hydroxyl group on the PEG chain. In some embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In some embodiments, the PEG-OH lipid includes one or more hydroxyl groups on the PEG chain. In some embodiments, a PEG-OH or hydroxy-PEGylated lipid comprises an —OH group at the terminus of the PEG chain. Each possibility represents a separate embodiment of the present invention.

In some embodiments, a PEG lipid useful in the present invention is a compound of Formula (PL-I). Provided herein are compounds of Formula (PL-I):

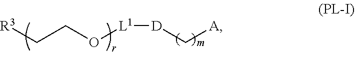

or salts thereof, wherein:
   R³ is —OR^{O};
   R^{O} is hydrogen, optionally substituted alkyl, or an oxygen protecting group;
   r is an integer between 1 and 100, inclusive;
   L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, —OC(O), OC(O)O, OC(O)N(R^{N}), NR^{N}C(O)O, or NR^{N}C(O)N(R^{N}),
   D is a moiety obtained by click chemistry or a moiety cleavable under physiological conditions;
   m is 0, 1, 2, 3, 4, 5, 6, 7, 8, 9, or 10;
   A is of the formula:

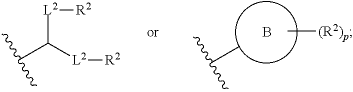

each instance of of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N}),
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), —C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S) NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In some embodiments, the compound of Formula (PL-I) is a PEG-OH lipid (i.e., R³ is —OR^{O}, and R^{O} is hydrogen). In some embodiments, the compound of Formula (PL-I) is of Formula (PL-I-OH):

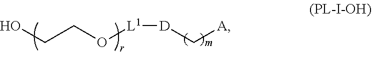

or a salt thereof.

In some embodiments, a PEG lipid useful in the present invention is a PEGylated fatty acid. In some embodiments, a PEG lipid useful in the present invention is a compound of Formula (PL-II). Provided herein are compounds of Formula (PL-II):

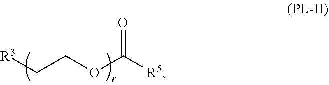

or a salt thereof, wherein:
   R³ is —OR^{O};
   R^{O} is hydrogen, optionally substituted alkyl or an oxygen protecting group;
   r is an integer between 1 and 100, inclusive;
   R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(O)₀, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
   each instance of R^{N} is independently hydrogen, optionally substituted alkyl, or a nitrogen protecting group.

In some embodiments, the compound of Formula (PL-II) is of Formula (PL-II-OH):

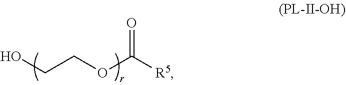

or a salt thereof, wherein:
   r is an integer between 1 and 100;
   R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}) C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
   each instance of R^{N} is independently hydrogen, optionally substituted alkyl, or a nitrogen protecting group.

In some embodiments, r is an integer between 10 to 80, between 20 to 70, between 30 to 60, or between 40 to 50.

In some embodiments, r is 45.

In some embodiments, R⁵ is C₁₇ alkyl.

In yet other embodiments the compound of Formula (PL-II) is:

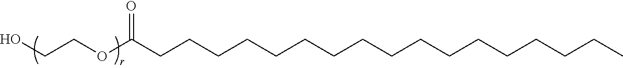

or a salt thereof.

In one embodiment, the compound of Formula (PL-II) is

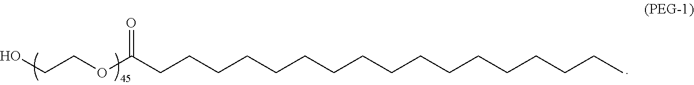

In some aspects, the lipid composition of the pharmaceutical compositions described herein does not comprise a PEG lipid.

In some embodiments, the PEG lipids may be one or more of the PEG lipids described in U.S. Application No. 62/520,530.

In some embodiments, the PEG lipid is a compound of Formula (PL-III):

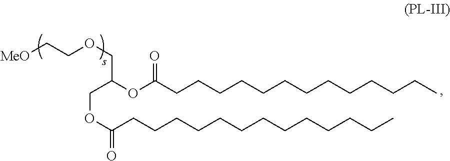

or a salt or isomer thereof, wherein s is an integer between 1 and 100.

In some embodiments, the PEG lipid is a compound of the following formula:

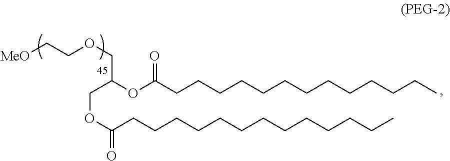

or a salt or isomer thereof.

### Structural Lipids

As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a mixture of two or more components each independently selected from cholesterol, fecosterol, sitosterol, ergosterol, campesterol, stigmasterol, brassicasterol, tomatidine, tomatine, ursolic acid, alpha-tocopherol, hopanoids, phytosterols, and steroids. In some embodiments, the structural lipid is a sterol. In some embodiments, the structural lipid is a mixture of two or more sterols. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the structural lipids may be one or more structural lipids described in U.S. Application No. 62/520,530.

As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the structural lipid is

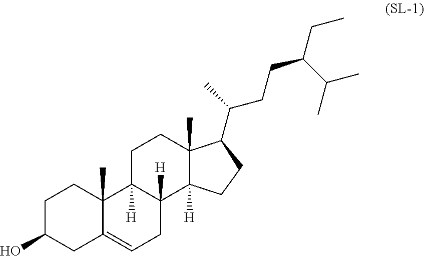

or a salt thereof.

In some embodiments, the structural lipid is SL-1.

In some embodiments, the structural lipid is

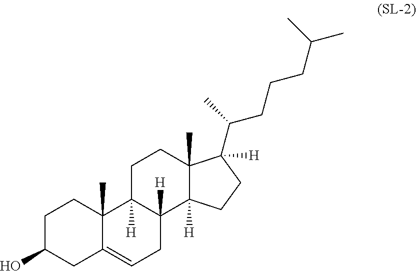

or a salt thereof.

In some embodiments, the structural lipid (e.g., SL-2) is present at a concentration ranging from about 15 mol % to about 70 mol %, from about 20 mol % to about 60 mol %, from about 25 mol % to about 50 mol %, from about 30 mol % to about 45 mol %, from about 35 mol % to about 40 mol %, or from about 36 mol % to about 38 mol %.

In some embodiments, the structural lipid (e.g., SL-2) is present at a concentration of about 36.6±25 mol %, about 36.6±20 mol %, about 36.6±15 mol %, about 36.6±10 mol %, about 36.6±9 mol %, about 36.6±8 mol %, about 36.6±7 mol %, about 36.6±6 mol %, about 36.6±5 mol %, about 36.6±4 mol %, about 36.6±3 mol %, about 36.6±2 mol %, about 36.6±1 mol %, about 36.6±0.8 mol %, about 36.6±0.6 mol %, about 36.6±0.5 mol %, about 36.6±0.4 mol %, about 36.6±0.3 mol %, about 36.6±0.2 mol %, or about 36.6±0.1 mol % (e.g., about 36.6 mol %).

### Encapsulation Agents

In some embodiments of the present disclosure, the encapsulation agent is a compound of Formula (EA-I):

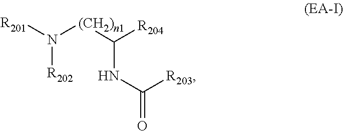

or salts or isomers thereof, wherein
   R₂₀₁ and R₂₀₂ are each independently selected from the group consisting of H, C₁-C₆ alkyl, C₂-C₆ alkenyl, and (C═NH)N(R₁₀₁)₂ wherein each R₁₀₁ is independently selected from the group consisting of H, C₁-C₆ alkyl, and C₂-C₆ alkenyl;
   R₂₀₃ is selected from the group consisting of C₁-C₂₀ alkyl and C₂-C₂₀ alkenyl;
   R₂₀₄ is selected from the group consisting of H, C₁-C₂₀ alkyl, C₂-C₂₀ alkenyl, C(O)(OC₁-C₂₀ alkyl), C(O)(OC₂-C₂₀ alkenyl), C(O)(NHC₁-C₂₀ alkyl), and C(O)(NHC₂-C₂₀ alkenyl);
   n1 is selected from 1, 2, 3, 4, 5, 6, 7, 8, 9, and 10.

In some embodiments, R₂₀₁ and R₂₀₂ are each independently selected from the group consisting of H and CH₃.

In some embodiments, R₂₀₁ and R₂₀₂ are each independently selected from the group consisting of (C═NH)NH₂ and (C═NH)N(CH₃)₂

In some embodiments, R₂₀₃ is selected from the group consisting of C₁-C₂₀ alkyl, C₈-C₁₈ alkyl, and C₁₂-C₁₆ alkyl.

In some embodiments, R₂₀₄ is selected from the group consisting of H, C₁-C₂₀ alkyl, C₂-C₂₀ alkenyl, C(O)(OC₁-C₂₀ alkyl), C(O)(OC₂-C₂₀ alkenyl), C(O)(NHC₁-C₂₀ alkyl), and C(O)(NHC₂-C₂₀ alkenyl); C₈-C₁₈ alkyl, C₈-C₁₈ alkenyl, C(O)(OC₈-C₁₈ alkyl), C(O)(OC₈-C₁₈ alkenyl), C(O)(NHC₈-C₁₈ alkyl), and C(O)(NHC₈-C₁₈ alkenyl); and C₁₂-C₁₆ alkyl, C₁₂-C₁₆ alkenyl, C(O)(OC₁₂-C₁₆ alkyl), C(O)(OC₁₂-C₁₆ alkenyl), C(O)(NHC₁₂-C₁₆ alkyl), and C(O)(NHC₁₂-C₁₆ alkenyl);

In some embodiments, n1 is selected from 1, 2, 3, 4, 5, 6, 7, 8, 9, and 10; n1 is selected from 1, 2, 3, 4, 5, and 6; n₁ is selected from 2, 3, and 4.

In some embodiments, n1 is 3.

In some embodiments of the present disclosure, the encapsulation agent is a compound of Formula (EA-II):

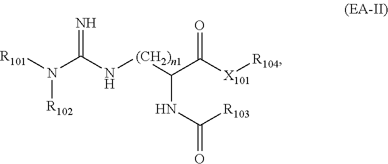

or salts or isomers thereof, wherein
X₁₀₁ is a bond, NH, or O;
R₁₀₁ and R₁₀₂ are each independently selected from the group consisting of H, C₁-C₆ alkyl, and C₂-C₆ alkenyl;
R₁₀₃ and R₁₀₄ are each independently selected from the group consisting of C₁-C₂₀ alkyl and C₂-C₂₀ alkenyl; and
n1 is selected from 1, 2, 3, 4, 5, 6, 7, 8, 9, and 10.

In some embodiments, X₁₀₁ is a bond.

In some embodiments, X₁₀₁ is NH.

In some embodiments, X₁₀₁ is O.

In some embodiments, R₁₀₁ and R₁₀₂ are each independently selected from the group consisting of H and CH₃.

In some embodiments, R₁₀₃ is selected from the group consisting of C₁-C₂₀ alkyl, C₈-C₁₈ alkyl, and C₁₂-C₁₆ alkyl.

In some embodiments, R₁₀₄ is selected from the group consisting of C₁-C₂₀ alkyl, C₈-C₁₈ alkyl, and C₁₂-C₁₆ alkyl.

In some embodiments, n1 is selected from 1, 2, 3, 4, 5, 6, 7, 8, 9, and 10; n1 is selected from 1, 2, 3, 4, 5, and 6; n1 is selected from 2, 3, and 4.

In some embodiments, n1 is 3.

Exemplary encapsulation agents include, but are not limited to, ethyl lauroyl arginate, ethyl myristoyl arginate, ethyl palmitoyl arginate, ethyl cholesterol-arginate, ethyl oleic arginate, ethyl capric arginate, and ethyl carprylic arginate.

In some embodiments, the encapsulation agent is ethyl lauroyl arginate,

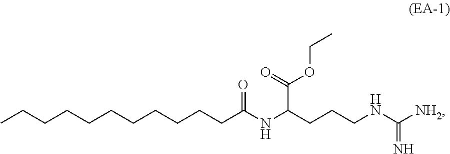 [SMILES: CCCCCCCCCCCC(=O)NC(CCCNC(=N)N)C(=O)OCC]

or a salt or isomer thereof.

In some embodiments, the encapsulation agent is at least one compound selected from the group consisting of;

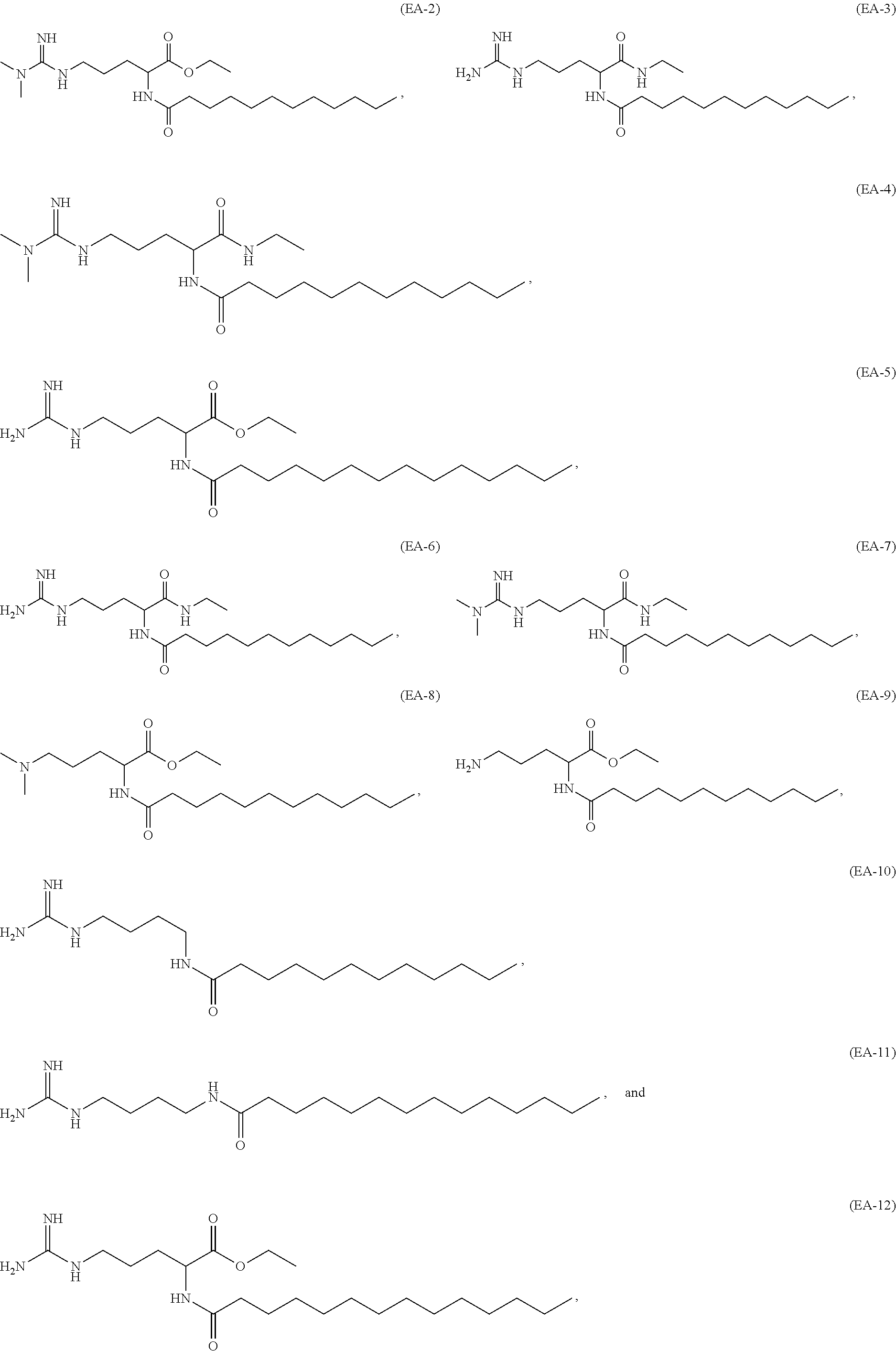

or salts and isomers thereof, such as, for example free bases, TFA salts, and/or HCl salts.

### Phospholipids

Phospholipids may assemble into one or more lipid bilayers. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyglycerols, and phosphatidic acids Phospholipids also include phosphosphingolipid, such as sphingomyelin.

In some embodiments, a phospholipid useful or potentially useful in the present invention is an analog or variant of DSPC. In some embodiments, a phospholipid useful or potentially useful in the present invention is a compound of Formula (PL-1):

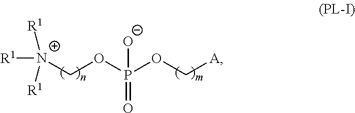

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

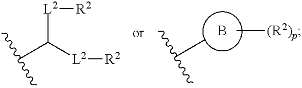

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—.
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)₀—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

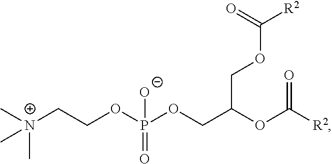

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

In some embodiments, the phospholipids may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, a LNP includes DSPC. In some embodiments, a LNP includes DOPE. In some embodiments, a LNP includes both DSPC and DOPE.

### i) Phospholipid Head Modifications

In some embodiments, a phospholipid useful or potentially useful in the present invention comprises a modified phospholipid head (e.g., a modified choline group). In some embodiments, a phospholipid with a modified head is DSPC, or analog thereof, with a modified quaternary amine. In some embodiments, in embodiments of Formula (PL-I), at least one of R¹ is not methyl. In some embodiments, at least one of R¹ is not hydrogen or methyl. In some embodiments, the compound of Formula (PL-I) is one of the following formulae:

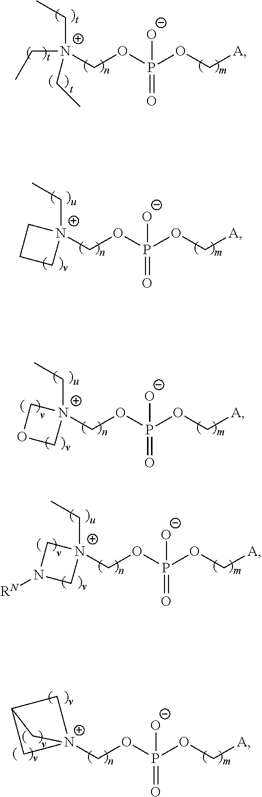

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In some embodiments, a compound of Formula (PL-I) is of Formula (PL-I-a):

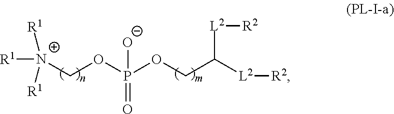

or a salt thereof.

In some embodiments, a phospholipid useful or potentially useful in the present invention comprises a cyclic moiety in place of the glyceride moiety. In some embodiments, a phospholipid useful in the present invention is DSPC, or analog thereof, with a cyclic moiety in place of the glyceride moiety. In some embodiments, the compound of Formula (PL-I) is of Formula (PL-I-b):

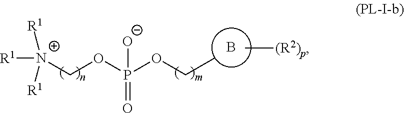

or a salt thereof.

### ii) Phospholipid Tail Modifications

In some embodiments, a phospholipid useful or potentially useful in the present invention comprises a modified tail. In some embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. In some embodiments, In some embodiments, the compound of (PL-I) is of Formula (PL-I-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R″)—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—.

In some embodiments, the compound of Formula (PL-I) is of Formula (PL-I-c):

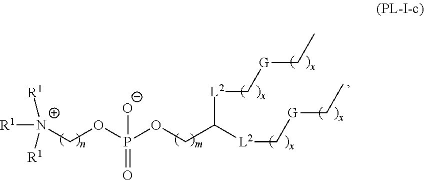

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)₀—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—. Each possibility represents a separate embodiment of the present invention.

In some embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in some embodiments, a phospholipid useful or potentially useful in the present invention is a compound of Formula (PL-1), wherein n is 1, 3, 4, 5, 6, 7, 8, 9, or 10. In some embodiments, a compound of Formula (PL-I) is of one of the following formulae:

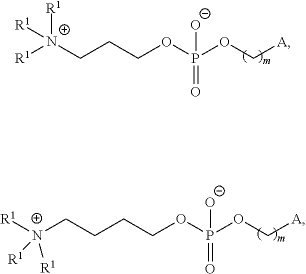

or a salt thereof.

### Alternative Lipids

In some embodiments, an alternative lipid is used in place of a phospholipid of the present disclosure. Non-limiting examples of such alternative lipids include the following:

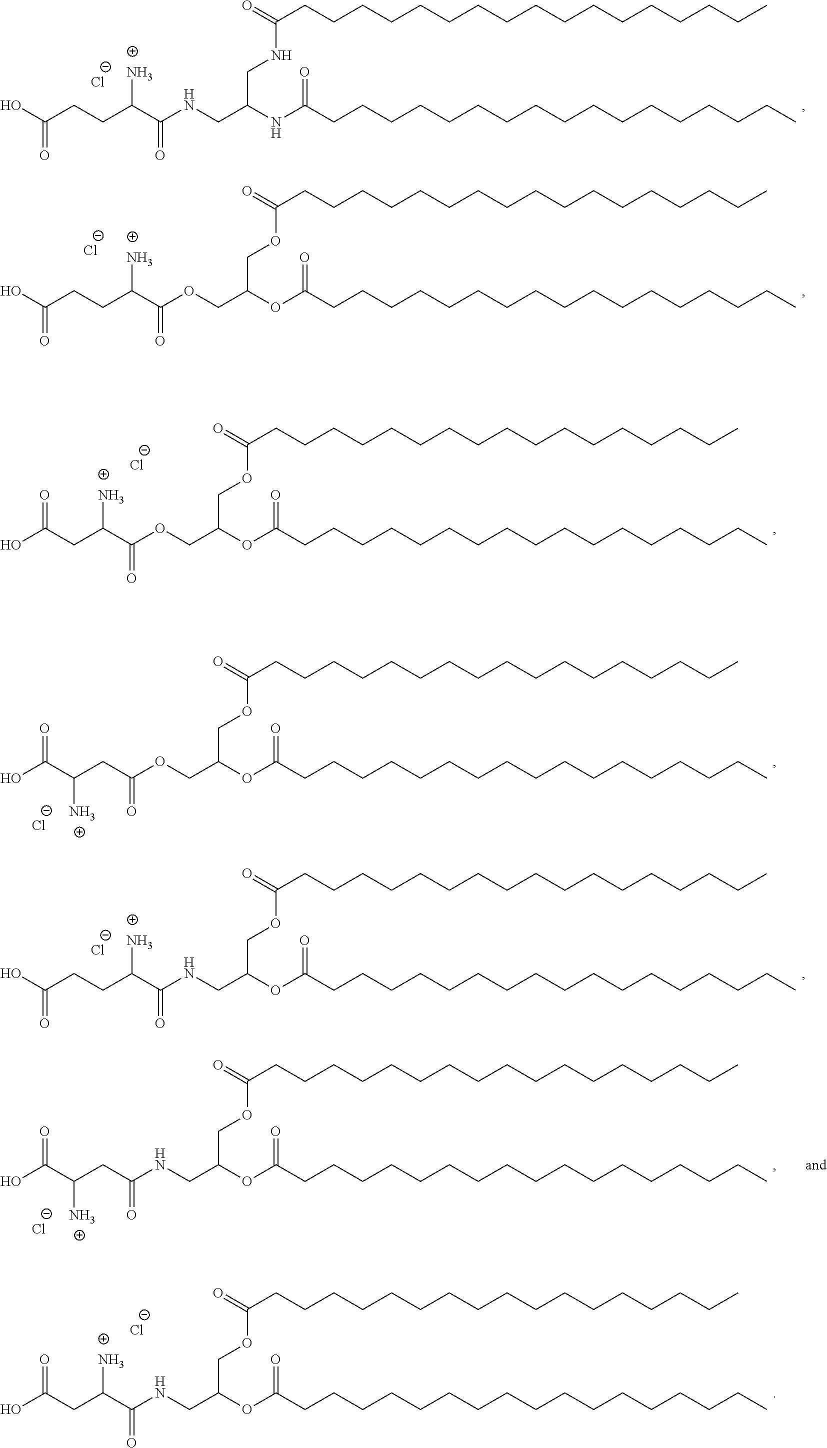

### Adjuvants

In some embodiments, a LNP that includes one or more lipids described herein may further include one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

### Therapeutic Agents

Lipid nanoparticles (e.g., empty LNPs or loaded LNPs) may include one or more therapeutic and/or prophylactics. The disclosure features methods of delivering a therapeutic and/or prophylactic to a mammalian cell or organ, producing a polypeptide of interest in a mammalian cell, and treating a disease or disorder in a mammal in need thereof comprising administering to a mammal and/or contacting a mammalian cell with a lipid nanoparticle (e.g., an empty LNP or a loaded LNP) including a therapeutic and/or prophylactic.

Therapeutic and/or prophylactics include biologically active substances and are alternately referred to as “active agents.” A therapeutic and/or prophylactic may be a substance that, once delivered to a cell or organ, brings about a desirable change in the cell, organ, or other bodily tissue or system. Such species may be useful in the treatment of one or more diseases, disorders, or conditions. In some embodiments, a therapeutic and/or prophylactic is a small molecule drug useful in the treatment of a particular disease, disorder, or condition.

In some embodiments, a therapeutic and/or prophylactic is a vaccine, a compound (e.g., a polynucleotide or nucleic acid molecule that encodes a protein or polypeptide or peptide or a protein or polypeptide or protein) that elicits an immune response, and/or another therapeutic and/or prophylactic. Vaccines include compounds and preparations that are capable of providing immunity against one or more conditions related to infectious diseases and can include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. In some embodiments, a vaccine and/or a compound capable of eliciting an immune response is administered intramuscularly via a composition of the disclosure.

In other embodiments, a therapeutic and/or prophylactic is a protein, for example a protein needed to augment or replace a naturally-occurring protein of interest. Such proteins or polypeptides may be naturally occurring, or may be modified using methods known in the art, e.g., modified so as to increase half life. Exemplary proteins are intracellular, transmembrane, or secreted proteins, peptides or polypeptides.

### Polynucleotides and Nucleic Acids

In some embodiments, the therapeutic agent is an agent that enhances (i.e., increases, stimulates, upregulates) protein expression. Non-limiting examples of types of therapeutic agents that can be used for enhancing protein expression include RNAs, mRNAs, dsRNAs, CRISPR/Cas9 technology, ssDNAs and DNAs (e.g., expression vectors). The agent that upregulates protein expression may upregulate expression of a naturally occurring or non-naturally occurring protein (e.g., a chimeric protein that has been modified to improve half life, or one that comprises desirable amino acid changes). Exemplary proteins include intracellular, transmembrane, or secreted proteins, peptides, or polypeptides.

In some embodiments, the therapeutic agent is a DNA therapeutic agent. The DNA molecule can be a double-stranded DNA, a single-stranded DNA (ssDNA), or a molecule that is a partially double-stranded DNA, i.e., has a portion that is double-stranded and a portion that is single-stranded. In some cases the DNA molecule is triple-stranded or is partially triple-stranded, i.e., has a portion that is triple stranded and a portion that is double stranded. The DNA molecule can be a circular DNA molecule or a linear DNA molecule.

A DNA therapeutic agent can be a DNA molecule that is capable of transferring a gene into a cell, e.g., a DNA molecule that encodes and can express a transcript. In other embodiments, the DNA molecule is a synthetic molecule, e.g., a synthetic DNA molecule produced in vitro. In some embodiments, the DNA molecule is a recombinant molecule. Non-limiting exemplary DNA therapeutic agents include plasmid expression vectors and viral expression vectors.

The DNA therapeutic agents described herein, e.g., DNA vectors, can include a variety of different features. The DNA therapeutic agents described herein, e.g., DNA vectors, can include a non-coding DNA sequence. For example, a DNA sequence can include at least one regulatory element for a gene, e.g., a promoter, enhancer, termination element, polyadenylation signal element, splicing signal element, and the like. In some embodiments, the non-coding DNA sequence is an intron. In some embodiments, the non-coding DNA sequence is a transposon. In some embodiments, a DNA sequence described herein can have a non-coding DNA sequence that is operatively linked to a gene that is transcriptionally active. In other embodiments, a DNA sequence described herein can have a non-coding DNA sequence that is not linked to a gene, i.e., the non-coding DNA does not regulate a gene on the DNA sequence.

In some embodiments, in the loaded LNP of the disclosure, the one or more therapeutic and/or prophylactic agents is a nucleic acid. In some embodiments, the one or more therapeutic and/or prophylactic agents is selected from the group consisting of a ribonucleic acid (RNA) and a deoxyribonucleic acid (DNA).

For example, in some embodiments, when the therapeutic and/or prophylactic agents is a DNA, the DNA is selected from the group consisting of a double-stranded DNA, a single-stranded DNA (ssDNA), a partially double-stranded DNA, a triple stranded DNA, and a partially triple-stranded DNA. In some embodiments, the DNA is selected from the group consisting of a circular DNA, a linear DNA, and mixtures thereof.

In some embodiments, in the loaded LNP of the disclosure, the one or more therapeutic and/or prophylactic agents is selected from the group consisting of a plasmid expression vector, a viral expression vector, and mixtures thereof.

For example, in some embodiments, when the therapeutic and/or prophylactic agents is a RNA, the RNA is selected from the group consisting of a single-stranded RNA, a double-stranded RNA (dsRNA), a partially double-stranded RNA, and mixtures thereof. In some embodiments, the RNA is selected from the group consisting of a circular RNA, a linear RNA, and mixtures thereof.

For example, in some embodiments, when the therapeutic and/or prophylactic agents is a RNA, the RNA is selected from the group consisting of a short interfering RNA (siRNA), an asymmetrical interfering RNA (aiRNA), a RNA interference (RNAi) molecule, a microRNA (miRNA), an antagomir, an antisense RNA, a ribozyme, a Dicer-substrate RNA (dsRNA), a small hairpin RNA (shRNA), a messenger RNA (mRNA), locked nucleic acids (LNAs) and CRISPR/Cas9 technology, and mixtures thereof.

For example, in some embodiments, when the therapeutic and/or prophylactic agents is a RNA, the RNA is selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.

In some embodiments, the one or more therapeutic and/or prophylactic agents is an mRNA. In some embodiments, the one or more therapeutic and/or prophylactic agents is a modified mRNA (mmRNA).

In some embodiments, the one or more therapeutic and/or prophylactic agents is an mRNA that incorporates a micro-RNA binding site (miR binding site). Further, in some embodiments, an mRNA includes one or more of stem loop, chain terminating nucleoside, polyA sequence, polyadenylation signal, and/or 5′ cap structure.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In some embodiments, all of a particular nucleobase type may be modified. In some embodiments, all uracils or uridines are modified. When all nucleobases, nucleosides, or nucleotides are modified, e.g., all uracils or uridines, the mRNA can be referred to as “fully modified”, e.g., for uracil or uridine.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,03′GpppG, m27,03′GppppG, m27,02′GppppG, m7Gpppm7G, m73′dGpppG, m27,03′GpppG, m27,03′GppppG, and m27,02′GppppG.

An mRNA may include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA.

An mRNA may include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In some embodiments, a stem loop may affect one or more function(s) of an mRNA, such as initiation of translation, translation efficiency, and/or transcriptional termination.

An mRNA may include a polyA sequence and/or polyadenylation signal. A polyA sequence may be comprised entirely or mostly of adenine nucleotides or analogs or derivatives thereof. A poly A sequence may also comprise stabilizing nucleotides or analogs. For example, a poly A sequence can include deoxythymidine, e.g., inverted (or reverse linkage) deoxythymidine (dT), as a stabilizing nucleotide or analog. Details on using inverted dT and other stabilizing poly A sequence modifications can be found, for example, in WO2017/049275 A2, the content of which is incorporated herein by reference. A polyA sequence may be a tail located adjacent to a 3′ untranslated region of an mRNA. In some embodiments, a polyA sequence may affect the nuclear export, translation, and/or stability of an mRNA.

An mRNA may include a microRNA binding site. MicroRNA binding sites (or miR binding sites) can be used to regulate mRNA expression in various tissues or cell types. In exemplary embodiments, miR binding sites are engineered into 3′ UTR sequences of an mRNA to regulate, e.g., enhance degradation of mRNA in cells or tissues expressing the cognate miR. Such regulation is useful to regulate or control “off-target” expression ir mRNAs, i.e., expression in undesired cells or tissues in viv. Details on using mir binding sites can be found, for example, in WO 2017/062513 A2, the content of which is incorporated herein by reference.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(tm5s2U), I-taurinomethyl-4-thio-pseudouridine, 5-methyl-uridine (m5U, i.e., having the nucleobase deoxythymine), 1-methyl-pseudouridine (m1ψ), 5-methyl-2-thio-uridine (m5s2U), 1-methyl-4-thio-pseudouridine (mls4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-I-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include a-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6±6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (m1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include a-thio-guanosine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ0), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, O6-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m1ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.) In some embodiments, the modified nucleobase is N1-methylpseudouridine (m1ψ) and the mRNA of the disclosure is fully modified with N1-methylpseudouridine (m1«). In some embodiments, N1-methylpseudouridine (m1ψ) represents from 75-100% of the uracils in the mRNA. In some embodiments, N1-methylpseudouridine (m1ψ) represents 100% of the uracils in the mRNA.

In some embodiments, the modified nucleobase is a modified cytosine.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m1ψ), 5-methoxy-uridine (mo5U), 5-methyl-cytidine (m5C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m5C). In some embodiments, the mRNA comprises 1-methyl-pseudouridine (m1ψ). In some embodiments, the mRNA comprises 1-methyl-pseudouridine (m1ψ) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises comprises N6-methyl-adenosine (m6A). In some embodiments, the mRNA comprises N6-methyl-adenosine (m6A) and 5-methyl-cytidine (m5C).

In some embodiments, an mRNA of the disclosure is uniformly modified (i.e., fully modified, modified through-out the entire sequence) for a particular modification. For example, an mRNA can be uniformly modified with N1-methylpseudouridine (m1ψ) or 5-methyl-cytidine (m5C), meaning that all uridines or all cytosine nucleosides in the mRNA sequence are replaced with N1-methylpseudouridine (m1ψ) or 5-methyl-cytidine (m5C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

The mmRNAs of the disclosure can include a combination of modifications to the sugar, the nucleobase, and/or the internucleoside linkage. These combinations can include any one or more modifications described herein.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In some embodiments, the mRNA sequence is optimized using optimization algorithms, e.g., to optimize expression in mammalian cells or enhance mRNA stability.

In some embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In some embodiments, mRNAs are made using IVT enzymatic synthesis methods. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In some embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme.

Either enzymatic or chemical ligation methods may be used to conjugate polynucleotides or their regions with different functional moieties, such as targeting or delivery agents, fluorescent labels, liquids, nanoparticles, etc. Therapeutic Agents for Reducing Protein Expression

In some embodiments, the therapeutic agent is a therapeutic agent that reduces (i.e., decreases, inhibits, downregulates) protein expression. Non-limiting examples of types of therapeutic agents that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

### Pharmaceutical Compositions

Formulations comprising lipid nanoparticles may be formulated in whole or in part as pharmaceutical compositions. Pharmaceutical compositions may include one or more lipid nanoparticles. In some embodiments, a pharmaceutical composition may include one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP in the formulation of the disclosure. An excipient or accessory ingredient may be incompatible with a component of a LNP of the formulation if its combination with the component or LNP may result in any undesirable biological effect or otherwise deleterious effect.

In some embodiments, one or more excipients or accessory ingredients may make up greater than 50% of the total mass or volume of a pharmaceutical composition including a LNP. In some embodiments, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition comprises between 0.1% and 100% (wt/wt) of one or more lipid nanoparticles. As another example, a pharmaceutical composition comprises between 0.1% and 15% (wt/vol) of one or more amphiphilic polymers (e.g., 0.5%, 1%, 2.5%, 5%, 10%, or 12.5% w/v).

In some embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In some embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C. −50° C., −60° C., −70° C., −80° C., −90° C., −130° C. or −150° C.).

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. In some embodiments, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In some embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay, silicates), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only.

In some embodiments, the solid compositions may optionally comprise opacifying agents and can be of a composition that they release the active ingredient(s) in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220, 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 sm to 500 pm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all, of the group members are present in, employed in, or otherwise relevant to a given product or process.

It is also noted that the term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein.

All cited sources, for example, references, publications, patent applications, databases, database entries, and art cited herein, are incorporated into this application by reference, even if not expressly stated in the citation. In case of conflicting statements of a cited source and the instant application, the statement in the instant application shall control.

The disclosure having been described, the following examples are offered by way of illustration and not limitation.

### Definitions

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Alkyl groups may be optionally substituted.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. In some embodiments, Cis alkenyl may include one or more double bonds. A Cis alkenyl group including two double bonds may be a linoleyl group. Alkenyl groups may be optionally substituted.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₁₈ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2-dihydronaphthyl groups. The term “cycloalkyl” as used herein means a non-aromatic carbocycle and may or may not include any double or triple bond. Unless otherwise specified, carbocycles described herein may be unsubstituted or substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein may be unsubstituted or substituted heterocycle groups, i.e., optionally substituted heterocycles.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. In some embodiments, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein may be unsubstituted or substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR are alkoxy groups that can be the same or different and R″ ″ is an alkyl or alkenyl group), a phosphate (e.g., P(O)₄³⁻), a thiol (e.g., —SH), a sulfoxide (e.g., —S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂, or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. In some embodiments, a C₁₋₆ alkyl group may be further substituted with one, two, three, four, five, or six substituents as described herein.

As used herein, the terms “approximately” and “about,” as applied to one or more values of interest, refer to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, when used in the context of an amount of a given compound in a lipid component of a LNP, “about” may mean+/−10% of the recited value. For instance, a LNP including a lipid component having about 40% of a given compound may include 30-50% of the compound.

As used herein, the term “compound,” is meant to include all isomers and isotopes of the structure depicted. “Isotopes” refers to atoms having the same atomic number but different mass numbers resulting from a different number of neutrons in the nuclei. In some embodiments, isotopes of hydrogen include tritium and deuterium. Further, a compound, salt, or complex of the present disclosure can be prepared in combination with solvent or water molecules to form solvates and hydrates by routine methods.

As used herein, the term “upon” intends to refer to the time point being after an action happens. For example, “upon administration” refers to the time point being after the action of administration.

As used herein, the term “contacting” means establishing a physical connection between two or more entities. In some embodiments, contacting a mammalian cell with a LNP means that the mammalian cell and a nanoparticle are made to share a physical connection. Methods of contacting cells with external entities both in vivo and ex vivo are well known in the biological arts. In some embodiments, contacting a LNP and a mammalian cell disposed within a mammal may be performed by varied routes of administration (e.g., intravenous, intramuscular, intradermal, and subcutaneous) and may involve varied amounts of lipid nanoparticles. Moreover, more than one mammalian cell may be contacted by a LNP.

As used herein, the term “comparable method” refers to a method with comparable parameters or steps, as of the method being compared (e.g., the producing the LNP formulation of the present disclosure). In some embodiments, the “comparable method” is a method with one or more of steps i), ia), iaa), ib), ii), iia), iib), iic), iid), and iie) of the method being compared. In some embodiments, the “comparable method” is a method without one or more of steps i), ia), iaa), ib), ii), iia), iib), iic), iid), and iie) of the method being compared. In some embodiments, the “comparable method” is a method without one or more of steps ia) and ib) of the method being compared. In some embodiments, the “comparable method” is a method employing a water-soluble salt of a nucleic acid. In some embodiments, the “comparable method” is a method employing an organic solution that does not comprise an organic solvent-soluble nucleic acid. In some embodiments, the “comparable method” is a method comprising processing the lipid nanoparticle prior to administering the lipid nanoparticle formulation.

As used herein, the term “delivering” means providing an entity to a destination. In some embodiments, delivering a therapeutic and/or prophylactic to a subject may involve administering a LNP including the therapeutic and/or prophylactic to the subject (e.g., by an intravenous, intramuscular, intradermal, or subcutaneous route). Administration of a LNP to a mammal or mammalian cell may involve contacting one or more cells with the lipid nanoparticle.

As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to the level of delivery of a therapeutic and/or prophylactic by a control nanoparticle to a target tissue of interest (e.g., MC3, KC2, or DLinDMA). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. It will be understood that the enhanced delivery of a nanoparticle to a target tissue need not be determined in a subject being treated, it may be determined in a surrogate such as an animal model (e.g., a rat model).

As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to an off-target tissue (e.g., mammalian spleen). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. In some embodiments, for renovascular targeting, a therapeutic and/or prophylactic is specifically provided to a mammalian kidney as compared to the liver and spleen if 1.5, 2-fold, 3-fold, 5-fold, 10-fold, 15-fold, or 20-fold more therapeutic and/or prophylactic per 1 g of tissue is delivered to a kidney compared to that delivered to the liver or spleen following systemic administration of the therapeutic and/or prophylactic. It will be understood that the ability of a nanoparticle to specifically deliver to a target tissue need not be determined in a subject being treated, it may be determined in a surrogate such as an animal model (e.g., a rat model).

As used herein, “encapsulation efficiency” refers to the amount of a therapeutic and/or prophylactic that becomes part of a LNP, relative to the initial total amount of therapeutic and/or prophylactic used in the preparation of a LNP. In some embodiments, if 97 mg of therapeutic and/or prophylactic are encapsulated in a LNP out of a total 100 mg of therapeutic and/or prophylactic initially provided to the composition, the encapsulation efficiency may be given as 97%.

As used herein, “encapsulation”, “encapsulated”, “loaded”, and “associated” may refer to complete, substantial, or partial enclosure, confinement, surrounding, or encasement. As used herein, “encapsulation” or “association” may refer to the process of confining an individual nucleic acid molecule within a nanoparticle and/or establishing a physiochemical relationship between an individual nucleic acid molecule and a nanoparticle. As used herein, an “empty nanoparticle” may refer to a nanoparticle that is substantially free of a therapeutic or prophylactic agent. As used herein, an “empty nanoparticle” may refer to a nanoparticle that is substantially free of a nucleic acid. As used herein, an “empty nanoparticle” may refer to a nanoparticle that consists substantially of only lipid components.

As used herein, “expression” of a nucleic acid sequence refers to translation of an mRNA into a polypeptide or protein and/or post-translational modification of a polypeptide or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

As used herein, a “lipid component” is that component of a lipid nanoparticle that includes one or more lipids. In some embodiments, the lipid component may include one or more cationic/ionizable, PEGylated, structural, or other lipids, such as phospholipids.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. In some embodiments, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. In some embodiments, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. In some embodiments, a modified nucleobase species may include one or more substitutions that are not naturally occurring.

As used herein, the “N:P ratio” is the molar ratio of ionizable (in the physiological pH range) nitrogen atoms in a lipid to phosphate groups in an RNA, e.g., in a LNP including a lipid component and an RNA.

As used herein, a “lipid nanoparticle” is a composition comprising one or more lipids. Lipid nanoparticles are typically sized on the order of micrometers or smaller and may include a lipid bilayer. Lipid nanoparticles, as used herein, unless otherwise specified, encompass lipid nanoparticles (LNPs), liposomes (e.g., lipid vesicles), and lipoplexes. In some embodiments, a LNP may be a liposome having a lipid bilayer with a diameter of 500 nm or less.

As used herein, “naturally occurring” means existing in nature without artificial aid.

As used herein, “patient” refers to a subject who may seek or be in need of treatment, requires treatment, is receiving treatment, will receive treatment, or a subject who is under care by a trained professional for a particular disease or condition.

As used herein, a “PEG lipid” or “PEGylated lipid” refers to a lipid comprising a polyethylene glycol component.

As used herein, a “polymeric lipid” refers to a lipid comprising repeating subunits in its chemical structure. In some embodiments, the polymeric lipid is a lipid comprising a polymer component. In some embodiments, the polymeric lipid is a PEG lipid. In some embodiments, the polymeric lipid is not a PEG lipid. In some embodiments, the polymeric lipid is Brij or OH-PEG-stearate.

The phrase “pharmaceutically acceptable” is used herein to refer to those compounds, materials, composition, and/or dosage forms which are, within the scope of sound medical judgment, suitable for use in contact with the tissues of human beings and animals without excessive toxicity, irritation, allergic response, or other problems or complication, commensurate with a reasonable benefit/risk ratio.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In some embodiments, the nonaqueous media are ether, ethyl acetate, ethanol, isopropanol, or acetonitrile. Lists of suitable salts are found in Remington's Pharmaceutical Sciences, 17^{1h} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). A phospholipid or an analog or derivative thereof may include choline. A phospholipid or an analog or derivative thereof may not include choline. Particular phospholipids may facilitate fusion to a membrane. In some embodiments, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the “polydispersity index” is a ratio that describes the homogeneity of the particle size distribution of a system. A small value, e.g., less than 0.3, indicates a narrow particle size distribution.

As used herein, an amphiphilic “polymer” is an amphiphilic compound that comprises an oligomer or a polymer. In some embodiments, an amphiphilic polymer can comprise an oligomer fragment, such as two or more PEG monomer units. In some embodiments, an amphiphilic polymer described herein can be PS 20.

As used herein, the term “polypeptide” or “polypeptide of interest” refers to a polymer of amino acid residues typically joined by peptide bonds that can be produced naturally (e.g., isolated or purified) or synthetically.

As used herein, an “RNA” refers to a ribonucleic acid that may be naturally or non-naturally occurring. In some embodiments, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. In some embodiments, an RNA may be a messenger RNA (mRNA). Translation of an mRNA encoding a particular polypeptide, for example, in vivo translation of an mRNA inside a mammalian cell, may produce the encoded polypeptide.

RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

As used herein, a “single unit dose” is a dose of any therapeutic administered in one dose/at one time/single route/single point of contact, i.e., single administration event.

As used herein, a “split dose” is the division of a single unit dose or total daily dose into two or more doses.

As used herein, a “total daily dose” is an amount given or prescribed in a 24 hour period. It may be administered as a single unit dose.

As used herein, the term “subject” refers to any organism to which a composition or formulation in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, “Tx” refers to the amount of time lasted for the nucleic acid integrity (e.g., mRNA integrity) of a LNP, LNP solution, lyophilized LNP composition, or LNP formulation to degrade to about X of the initial integrity of the nucleic acid (e.g., mRNA) used for the preparation of the LNP, LNP solution, lyophilized LNP composition, or LNP formulation. For example, “T_{80%}” refers to the amount of time lasted for the nucleic acid integrity (e.g., mRNA integrity) of a LNP, LNP solution, lyophilized LNP composition, or LNP formulation to degrade to about 80% of the initial integrity of the nucleic acid (e.g., mRNA) used for the preparation of the LNP, LNP solution, lyophilized LNP composition, or LNP formulation. For another example, “T_{1/2}” refers to the amount of time lasted for the nucleic acid integrity (e.g., mRNA integrity) of a LNP, LNP solution, lyophilized LNP composition, or LNP formulation to degrade to about ½ of the initial integrity of the nucleic acid (e.g., mRNA) used for the preparation of the LNP, LNP solution, lyophilized LNP composition, or LNP formulation.

As used herein, “targeted cells” refers to any one or more cells of interest. The cells may be found in vitro, in vivo, in situ, or in the tissue or organ of an organism. The organism may be an animal. In some embodiments, the organism is a mammal. In some embodiments, the organism is a human. In some embodiments, the organism is a patient.

As used herein, “target tissue” refers to any one or more tissue types of interest in which the delivery of a therapeutic and/or prophylactic would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, the term “transfection” refers to the introduction of a species (e.g., an RNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. In some embodiments, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “zeta potential” refers to the electrokinetic potential of a lipid, e.g., in a particle composition.

As used herein, the term “polydispersity”, “polydispersity index”, or “PDI” refers to a measurement of the distribution of molecular mass in a given sample. The polydispersity is calculated as M_{w}/Mₙ, in which M_{w} is the mass-average molar mass (or molecular weight) and Mₙ is the number-average molar mass (or molecular weight).

The term, “empty lipid nanoparticle” or “empty LNP”, as used herein, refers to a lipid nanoparticle which is substantially free of therapeutic or prophylactic agent. In some embodiments, therapeutic or prophylactic agent is nucleic acid (e.g., mRNA). In some embodiments, the empty LNP is substantially free of nucleic acid (e.g., mRNA). In some embodiments, the empty LNP comprises an ionizable lipid, a phospholipid, a structural lipid, and a PEG lipid. In some embodiments, the empty LNP comprises substantially less nucleic acid (e.g., RNA) as compared to the loaded LNP. In some embodiments, the empty LNP comprises less than about 5% w/w, less than about 4% w/w, less than 3% w/w, less than 2% w/w, less than 1% w/w, less than 0.5% w/w, less than 0.4% w/w, less than 0.3% w/w, less than 0.2% w/w, or less than 0.1% w/w of nucleic acid (e.g., RNA). In some embodiments, the empty LNP is free of nucleic acid (e.g., mRNA). In some embodiments, the empty LNP is further substantially free of nucleic acid (e.g., mRNA) associated with the surface of the LNP or conjugated to the exterior of the LNP.

The term, “loaded lipid nanoparticle” or “loaded LNP” or “fLNP”, as used herein, refers to a lipid nanoparticle comprising a substantial amount of therapeutic or prophylactic agent. In some embodiments, therapeutic or prophylactic agent is nucleic acid (e.g., mRNA). In some embodiments, the loaded LNP comprises a substantial amount of nucleic acid (e.g., mRNA). In some embodiments, the empty LNP comprises an ionizable lipid, a phospholipid, a structural lipid, and a PEG lipid. In some embodiments, the empty LNP comprises a substantial amount of nucleic acid (e.g., mRNA) that is at least partially in the interior of the LNP. In some embodiments, the empty LNP comprises a substantial amount of nucleic acid (e.g., mRNA) that is associated with the surface of the LNP or conjugated to the exterior of the LNP.

It is understood that some properties of LNPs disclosed herein may be characterized by capillary zone electrophoresis (CZE). Capillary zone electrophoresis (CZE) refers to a separation technique which uses high voltage across a capillary to separate charged species based on their electrophoretic mobility. In some embodiments, the CZE is conducted with an acetate buffer (e.g., 50 mM sodium acetate at pH 5). In some embodiments, the CZE is conducted with a reverse voltage of about 10 kV across a 75 um capillary of 20 cm effective length. In some embodiments, the capillary is coated with polyethyleneimine.

The term “mobility peak”, as used herein, refers to a peak representing the distribution of a substance (e.g., a population of LNPs) as measured by CZE. In some embodiments, the intensity of the mobility peak is detected by scattered light. It is understood that the intensity of the peak may indicate the amount of the portion of the substance at the position of the peak. In some embodiments, the position of the peak is calculated against a neutral reference standard (e.g., DMSO) being characterized by a mobility peak at 0, and a charged reference standard (e.g., benzylamine) being characterized by a mobility peak at 1.0. In some embodiments, a population of LNPs may exhibit more than one peaks as measured by CZE, and unless indicated otherwise, the mobility peak refers to the peak having the greatest peak area among the more than one peaks.

The term “spread”, as used herein, refers to the width at half height of a peak (e.g., a mobility peak).

It is understood that, unless specified otherwise, the term “substantial portion”, as used herein, refers to a portion of at least about 50%. In some embodiments, the substantial portion is at least about 60%, at least about 70%, at least about 75%, at least about 80%, at least about 85%, at least about 88%, at least about 90%, at least about 91%, at least about 92%, at least about 93%, at least about 94%, at least about 95%, at least about 96%, at least about 97%, at least about 98%, or at least about 99%.

It is understood that some properties of LNPs disclosed herein may be characterized by asymmetric flow field flow fractionation (AF4). AF4 refers to a one phase separation that uses a perpendicular flow against a membrane (cross-flow) in conjunction with a channel flow parallel to the membrane to fractionate samples based on their diffusion behavior. The channel flow gives a parabolic profile and the perpendicular flow drives macromolecules toward the boundary layer of the membrane. Diffusion related to Brownian motions moves smaller particles with higher diffusion rates higher in the channel where longitudinal flow is faster, eluting smaller particles more quickly. In some embodiments, this technique is coupled to a separation to convolute the polydispersity of LNPs.

The term “size-heterogeneity mode peak” or “Rg mode peak”, as used herein, refers to a peak representing the distribution of a substance (e.g., a population of LNPs) as measured by AF4. In some embodiments, the intensity of the mobility peak is detected by scattered light, UV, or RI. It is understood that the intensity of the peak may indicate the amount of the portion of the substance at the position of the peak. In some embodiments, a population of LNPs may exhibit more than one peaks as measured by AF4 and unless indicated otherwise, the size-heterogeneity mode peak refers to the peak having the greatest peak area among the more than one peaks.

The term “distribution percentage”, as used herein, refers to the percentage of the peak area of a referenced peak over the total peak area of all peaks in a spectrum or diagram. For example, the distribution percentage of a mobility peak refers the percentage of the peak area of the mobility peak over the total peak area of all peaks of a substance (e.g., a population of LNPs) as measured by CZE. For another example, the distribution percentage of a size-heterogeneity mode peak refers to the percentage of the peak are of the size-heterogeneity mode peak over the total peak area of all peaks of a substance (e.g., a population of LNPs) as measured by AF4.

The term “radius of gyration”, as used herein, refers to the radial distance to a point which would have a moment of inertia the same as the body's actual distribution of mass, if the total mass of the body were concentrated there. In some embodiments, the radius of gyration is measured by AF4.

The term “free of”, as used herein, means not comprising the referenced component. For example, when a population, solution, or formulation is described as being “free of PEG lipid”, the population, solution, or formulation does not comprise PEG lipid (e.g., does not comprise a PEG lipid described herein (e.g., does not comprise PEG-DMG)).

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

It is understood that all values presented in the example are approximate, and are subject to instrumental and/or experimental variations.

### Materials and Equipments

### Raw Materials

TABLE 1
Lipid composition.

| Lipid | Molecular Weight (g/mol) | Mol % |
|---|---|---|
| SM102 | 710.2 | 49.0 |
| DSPC | 790.2 | 11.2 |
| Cholesterol | 386.7 | 39.3 |
| PEG2000-DMG | 2510 | 0.5 |

TABLE 2
Lipid stock details.

| | |
|---|---|
| Total Molar [Lipid] (mM) | 40.0 |
| Total Mass [Lipid] (mg/mL) | 24.0 |
| Average Molecular Weight (g/mol) | 601 |

TABLE 3
Process buffer.

| Buffer | Purpose(s) |
|---|---|
| 5 mM Sodium Acetate pH 5.0 | Aqueous Mixing Stream, |
| | Inline Dilution, Dialysis |

### Equipments

Benchtop scale equipment. The mixing system utilized LD300 pumps for the aqueous buffer and organic Lipid Stock Solution streams (LSS). 0.3, 0.5, 1 and 2 mm V-Mixer mixers and 0.5, 1, 1.5 and 2 mm T-Mixer were used for nanoprecipitation reactions. All tubing assemblies were constructed with Size 16 (⅛ inch inner diameter) Chemdurance tubing. A Masterflex peristaltic pump was implemented for the inline dilution stream downstream of the mixer.

Skid level 2 mm mixer equipment. The mixing system utilized two Watson Marlow pumps for aqueous buffer, organic and in-line dilution streams. 2 mm V-Mixer, 2 mm T-Mixer and 1.7 mm T-Mixer were used for nanoprecipitation reaction. All tubing assemblies were constructed with Size 36 Masterflex tubing (tubing ID 9.7 mm).

Pilot lab 3-4 mm mixer equipment. Nanoprecipitation is performed using either the 4 mm V-Mixer, 4 mm T-Mixer or the 3 mm T-Mixer.

The mixing system utilizes the Watson-Marlow (WM) pumps with max flow rate up to 2300 mL/min per pump using the current tubing dimension for lipid stock solution (LSS) and QF1200 pumps for the aqueous buffer (AQ) For a flow through of 5333 mL/min, one WM pump is needed for the LSS stream and one QF1200 pump is needed for the AQ stream.

The post-nanoprecipitation residence time tubing is size 36 (tubing ID 9.7 mm) Masterflex tubing. ID of 9.7 mm corresponds to (0.97/2)²*π=0.739 cm² of cross-sectional area. 5333 mL/min flow rate runs at 120 cm/s velocity through this area. A total of 6 meter of pre-inline-dilution (ILD) tubing is needed for the 5 seconds of pre-ILD flow through of 5333 mL/min. A QF1200 pump is required for the ILD stream, which mixes downstream with the nanoprecipitation flow-through.

Dialysis was performed using 3 mL 10 kD Slide-A-Lyzer cassettes.

Dynamic Light Scattering (DLS) measurements were obtained from a Wyatt Dynapro 2 instrument. The rest of the analytical experiment is performed at the AD group.

### Experimental Design

### Benchtop Scale Design

Benchtop-scale nanoprecipitation using up to 2 mm mixers were performed using flow rate as shown in Table 4 and Table 5. Flow ratio of 3:7 of LSS:aqueous within a mixer (30% LSS) was used for all V-Mixer runs, per operation conditions of the legacy V-Mixer processes. Flow ratio of 1:3 of LSS:aqueous within a mixer (25% LSS) was used for all T-Mixer runs.

TABLE 4
Nanoprecipitation parameters for benchtop V-Mixer runs

| | Mixer | | | | |
|---|---|---|---|---|---|
| | outlet | Total | Ethanol | Aqueous | ILD |
| Experiment | diameter | flow | flow | flow | flow |
| No. | (mm) | (mL/min) | (mL/min) | (mL/min) | (mL/min) |
| 1 | 0.3 | 30 | 9 | 21 | 42 |
| 2 | 0.3 | 48 | 14 | 34 | 67 |
| 3 | 0.3 | 64 | 19 | 45 | 90 |
| 4 | 0.3 | 78 | 23 | 55 | 109 |
| 5 | 0.3 | 90 | 27 | 63 | 126 |
| 6 | 0.5 | 5 | 1.5 | 3.5 | 7 |
| 7 | 0.5 | 10 | 3 | 7 | 14 |
| 8 | 0.5 | 50 | 15 | 35 | 70 |
| 9 | 0.5 | 70 | 21 | 49 | 98 |
| 10 | 0.5 | 100 | 30 | 70 | 140 |
| 11 | 0.5 | 80 | 24 | 56 | 112 |
| 12 | 0.5 | 107 | 32 | 75 | 150 |
| 13 | 0.5 | 130 | 39 | 91 | 182 |
| 14 | 0.5 | 180 | 54 | 126 | 252 |
| 15 | 1 | 5 | 1.5 | 3.5 | 7 |
| 16 | 1 | 10 | 3 | 7 | 14 |
| 17 | 1 | 50 | 15 | 35 | 70 |
| 18 | 1 | 100 | 30 | 70 | 140 |
| 19 | 1 | 200 | 60 | 140 | 280 |
| 20 | 1 | 160 | 48 | 112 | 224 |
| 21 | 1 | 214 | 64 | 150 | 300 |
| 22 | 1 | 260 | 78 | 182 | 364 |
| 23 | 1 | 300 | 90 | 210 | 420 |
| 24 | 1 | 360 | 108 | 252 | 504 |
| 25 | 2 | 320 | 96 | 224 | 448 |
| 26 | 2 | 429 | 129 | 300 | 600 |
| 27 | 2 | 200 | 60 | 140 | 280 |

TABLE 5
Nanoprecipitation parameters for benchtop T-Mixer runs

| | Mixer | | | | |
|---|---|---|---|---|---|
| | outlet | Total | Ethanol | Aqueous | ILD |
| Experiment | diameter | flow | flow | flow | flow |
| No. | (mm) | (mL/min) | (mL/min) | (mL/min) | (mL/min) |
| 1 | 0.5 | 10 | 2.5 | 7.5 | 14 |
| 2 | 0.5 | 20 | 5 | 15 | 28 |
| 3 | 0.5 | 50 | 12.5 | 37.5 | 70 |
| 4 | 0.5 | 100 | 25 | 75 | 140 |
| 5 | 0.5 | 200 | 50 | 150 | 280 |
| 6 | 1 | 10 | 2.5 | 7.5 | 14 |
| 7 | 1 | 20 | 5 | 15 | 28 |
| 8 | 1 | 50 | 12.5 | 37.5 | 70 |
| 9 | 1 | 100 | 25 | 75 | 140 |
| 10 | 1 | 200 | 50 | 150 | 280 |
| 11 | 1.5 | 10 | 2.5 | 7.5 | 14 |
| 12 | 1.5 | 20 | 5 | 15 | 28 |
| 13 | 1.5 | 50 | 12.5 | 37.5 | 70 |
| 14 | 1.5 | 100 | 25 | 75 | 140 |
| 15 | 1.5 | 200 | 50 | 150 | 280 |
| 16 | 2 | 10 | 2.5 | 7.5 | 14 |
| 17 | 2 | 20 | 5 | 15 | 28 |
| 18 | 2 | 50 | 12.5 | 37.5 | 70 |
| 19 | 2 | 100 | 25 | 75 | 140 |
| 20 | 2 | 200 | 50 | 150 | 280 |

### Skid Level 2 mm Mixer Design

The same flow rates were maintained for all the mixers involved. LSS of 281 mL/min, aqueous buffer of 844 mL/min and Pre-ILD buffer of 1575 mL/min were used and all supplied by Watson-Marlow pumps. 1.5, 1.7, and 2 mm T-Mixer were used and one 2 mm V-Mixer run was also performed as a control study. The 2 mm and 1.7 mm T-Mixer were also repeated, where an in-line static mixer was added immediately at the downstream of the mixing T-Mixer to see if that improved the mixing quality of T-Mixer products.

### Pilot Lab 3-4 mm Mixer Design

This experiment aims to compare the mixing performance of 4 mm V-Mixer vs 4 mm T-Mixer and 3 mm T-Mixer at the next level production scale. Total flow rate is the same for all three mixers to compare at the same level. The choice of flow rate is decided upon three factors: predicted ethanol drop time and therefore size of eLNP, predicted backpressure during nanoprecipitation, and the capacity of the WM pump.

Both ethanol drop time and backpressure during nanoprecipitation can be predicted using CFD modeling. Backpressure could ideally be kept below 40 psi for the right operation of WM pumps as well as to prevent out-gasing, which leads to eLNP aggregation at air-water interface.

A WM pump can run up to 2000 mL/min at the highest RPM using current tubing setting in the pilot lab. 1333 mL/min is chosen to be the maximum flow rate adopted by one WM pump to ensure accurate tracking of flow velocity. Flow in the AQ inlet runs at a 3:1 volume flow ratio to the LSS stream. Therefore, AQ flow rate is 4000 mL/min by one QF1200 pump. LSS flow is 1333 mL/min, supplied by one WM pump.

The operating parameters for nanoprecipitation are listed in Table 6. If the pressure is too large to run at a total flow of 5333 mL/min, especially for 4 mm V-Mixer, a lower total flow rate would be used at 4000 mL/min, 3600 mL/min or 3000 mL/min. The prediction of those is also listed in Table 6.

TABLE 6
Nanoprecipitation parameters for pilot lab large runs

| | | | | | Predicted | Predicted | | | |
|---|---|---|---|---|---|---|---|---|---|
| | | | | | aqueous | ethanol | | | |
| | | Ethanol | Aqueous | ILD | pressure | pressure | Calculated | ethanol | |
| Experiment | | flow | flow | flow | w/o outlet | w/o outlet | Pre-ILD | drop | predicted |
| No. | Mixer type | (mL/min) | (mL/min) | (mL/min) | (psi) | (psi) | time (s) | time(s) | size(nm) |
| 1 | 4 mm | 1000 | 3000 | 5600 | 15 | 15 | 7 | 0.0158 | 55 |
| | V-Mixer | | | | | | | | |
| 2 | 4 mm | 900 | 2700 | 5040 | 12 | 12 | 7 | 0.0175 | 55 |
| | V-Mixer | | | | | | | | |
| 3 | 4 mm | 750 | 2250 | 4200 | 8 | 8 | 9 | 0.0207 | 60 |
| | V-Mixer | | | | | | | | |
| 4 | 3 mm | 1000 | 3000 | 5600 | 13 | 10 | 7 | 0.0016 | 35 |
| | T-Mixer | | | | | | | | |
| 5 | 3 mm | 1333 | 4000 | 7466 | 22 | 17 | 5 | 0.0013 | 35 |
| | T-Mixer | | | | | | | | |
| 6 | 4 mm | 1000 | 3000 | 5600 | 6 | 3 | 7 | 0.0064 | 45 |
| | T-Mixer | | | | | | | | |
| 7 | 4 mm | 1333 | 4000 | 7466 | 10 | 6 | 5 | 0.0048 | 40 |
| | T-Mixer | | | | | | | | |

### Exemplary Processes

### Simulation Set-Up

V-Mixer and T-Mixer of different outlet dimensions are divided into individual meshing to get the numerical solution of Navier-Stokes equation at locations within the fluid mixing domain. A system converged at steady state gives the pressure and ethanol content contour within the mixer.

As shown in FIG. 1, after simulation gets converged at the steady state, massless particles are injected across the ethanol inlet, together with the ethanol flow. The local fraction of ethanol was monitored along the increase of residence time. For lipid dissolved in ethanol stream, the local ethanol mass concentration starts at 100%, starts drop in the mixing domain and left at 20.4% at the outlet (25% volume ethanol). “Ethanol drop time” or At, is defined as the average time differences between where the local ethanol mass fraction is 99.9/(mixing starts) and 30.0% (defined here as the close to the end of mixing, treated as the end of nanoprecipitation), as experienced by all particles.

### Pilot Lab Water Preparation Run

Before the day of experiment, LSS and buffer were prepared, the TFF tubing was set up and filtration plate was rinsed in the pilot lab.

To prevent system leakage and excessively high backpressure, a water run is performed first to validate the nanoprecipitation system set-up. The same flow rate is followed as in FIG. 1, except that all stream runs using water. Flow velocity and pressure at each point is recorded and compared to the simulation value. If backpressure on the WM pump is larger than 40 psi, a lower flow rate approach will be adopted for a specific mixer.

### Nanoprecipitation

The nanoprecipitation process entails an aqueous (5 mM sodium acetate pH 5.0) and organic (40 mM LSS in ethanol) stream entering the V-Mixer or T-Mixer. Ethanol stream rapidly mixes with water within the mixer to achieve supersaturation of dissolved lipid in the ethanol stream. Lipid precipitates upon supersaturation to form the matrix of eLNPs. A discrete residence time of the intermediate product between the mixing reaction and inline dilution is defined by a specified length of tubing. Additional 5 mM sodium acetate pH 5.0 is added in an inline dilution step to reduce ethanol concentration in the aqueous environment and slow growth of the intermediate LNP. This reaction occurs under ambient conditions, typically between 15-25° C. 40 mM SM102:DSPC:Cholesterol:PEG2000-DMG lipid stock solution in ethanol was prepared as described in Table 1 and Table 2.

### Processing

TFF membranes (mPES, 0.02 m² surface area) was firstly wetted with 0.25 N NaOH, followed by water neutralization and then filled with running buffer (12.5% ethanol in 5 mM acetate buffer). TFF plates are fixed with torque wrench at 140 inch·pound. TFF was performed with a feeding rate of80 mL/min with no manual adjustment of the transmembrane pressure (TMP)

The process started with 1500 mL of 2.5 mg/mL eLNP suspension. It was concentrated 12× to 30 mg/mL (125 mL remaining), and then diafiltrated 8 times (1000 mL of buffer needed). The experiment then proceeded to concentrate further to 140 mg/mL of lipid, corresponding to 47× of initial concentration.

In a previous experiment, the hold-up lipid mass in this filter with a 700 mL of 3 mg/mL starting lipid concentration is 750 mg. Assume this hold-up mass applied in this experiment where we started with 900 mL of eLNP suspension, the mass of lipid remaining will be 2.5*1500−750=3000 mg, corresponding to an 80% yield. Hence the target final TFF harvest is 26 mL. Given the hold-up volume due to tubing is around 10 mL, TFF would stop at ˜16 mL benchmark in its last stage.

The TFF harvest was sent to ATO for lipid quantification analysis. The dilution protocol details were decided based on the true lipid concentration of those samples. 700 mg/mL sucrose buffer in 5 mM acetate buffer was pre-made. After sucrose spike and dilution, the final eLNP suspension before PHL is 74.5 mg/mL lipid and 200 mg/mL sucrose.

0.2 μm filter, 1.5 mL/min rate was used for filtration and clarification, and the pressure during clarification is monitored.

### Characterization and Analytical Experiments

Post-TFF eLNPs both from the large scaled 4 mm V-Mixer, 3 mm T-Mixer and 4 mm T-Mixer runs and from the SDM 0.5 mm V-Mixer and 0.5 mm T-Mixer runs are passed onto the analytical development team for a list of characterization tests, as following:
AF4 is to be run to analyze the detailed size population distribution
Flow cytometry to determine the existence of large subvisible groups
CZE to determine if there is any difference of charged group distribution
Cryo-EM to compare the size and morphology for eLNPs made using large V-Mixer and large T-Mixer.

### Storage Stability and Filtration Performance Studies

Previous SM102 data shows eLNP size increases around 0.25 nm per day at both 5° C. and room temperature. However, filtration performance decays after storage at 5° C., while generally better for room temperature.

Post-TFF eLNP after sucrose spike and clarification is stored at 5° C. At time=0, 3 days, 1 week and 2 weeks, 8 mL of eLNP suspension is filtrated using the 0.2 pm filter at 1.5 mL/min. Pressure variation over time of filtration is recorded and compared, as an indication of filtration performance/stability variation over 5° C. storage.

### Loading Experiments

Post-hoc loading (PHL) is performance for eLNPs made using 4 mm V-Mixer, 4 mm T-Mixer, 3 mm T-Mixer as well as the SDM experiments using 0.5 mm V-Mixer and 0.5 mm T-Mixer.

1.61 mg/mL mRNA in 32.5 mM acetate, 120 mM Tris pH 8.3 323 mg/mL sucrose and 20 mM Tris pH 7.5 4.5 mg/mL PEG2k-DMG were all premade. 5 mL of eLNP with sucrose in 5 mM acetate buffer from previous step was taken and mixed with 12.5 mL of mRNA solution (14.3 mL/min for eLNP and 35.7 mL/min for mRNA). The mixing is done using a 0.5 mm mixer, where mRNA stream went through the axial inlet and eLNP stream went through the two peripheral inlets. Neutralized buffer and PEG buffer was added in batches after PHL.

The final mRNA concentration is 0.82 mg/mL, the lipid concentration is 15.2 mg/mL and the final N:P ratio is 5.2.

### Results of Exemplary Processes

Benchtop Nanoprecipitation eLNP Size Summary

The detailed size information for benchtop V-Mixer nanoprecipitation is shown in Table 7.

TABLE 7
Benchtop nanoprecipitation using V-Mixer sizing results

| | Mixer outlet | Total | | | Ethanol |
|---|---|---|---|---|---|
| Experiment | diameter | flow | Size | | drop time |
| No. | (mm) | (mL/min) | (nm) | PDI | (s) |
| 1 | 0.3 | 30 | 36.0 | 0.083 | 0.0018 |
| 2 | 0.3 | 48 | 34.1 | 0.141 | 0.0004 |
| 3 | 0.3 | 64 | 32.6 | 0.139 | 0.0005 |
| 4 | 0.3 | 78 | 31.5 | 0.145 | 0.0003 |
| 5 | 0.3 | 90 | 34.1 | 0.222 | 0.0002 |
| 6 | 0.5 | 5 | 202.2 | 0.258 | |
| 7 | 0.5 | 10 | 116.9 | 0.258 | |
| 8 | 0.5 | 50 | 40.9 | 0.140 | 0.0041 |
| 9 | 0.5 | 70 | 37.0 | 0.120 | 0.0043 |
| 10 | 0.5 | 80 | 35.0 | 0.115 | |
| 11 | 0.5 | 100 | 33.0 | 0.147 | 0.0013 |
| 12 | 0.5 | 107 | 33.1 | 0.190 | |
| 13 | 0.5 | 130 | 32.2 | NA | 0.0010 |
| 14 | 0.5 | 180 | 32.9 | 0.273 | |
| 15 | 1 | 5 | 272.3 | 0.177 | |
| 16 | 1 | 10 | 203.3 | 0.248 | |
| 17 | 1 | 50 | 67.6 | 0.279 | |
| 18 | 1 | 100 | 53.2 | 0.189 | 0.0087 |
| 19 | 1 | 200 | 43.6 | 0.181 | 0.0052 |
| 20 | 1 | 160 | 45.6 | 0.112 | 0.0038 |
| 21 | 1 | 214 | 41.3 | 0.095 | 0.0014 |
| 22 | 1 | 260 | 41.5 | 0.135 | 0.0066 |
| 23 | 1 | 300 | 40.3 | 0.130 | 0.0050 |
| 24 | 1 | 360 | 38.8 | 0.132 | 0.0043 |
| 25 | 2 | 320 | 57.4 | 0.155 | 0.0526 |
| 26 | 2 | 429 | 49.4 | 0.149 | 0.0175 |
| 27 | 2 | 200 | 67.8 | 0.143 | 0.0089 |

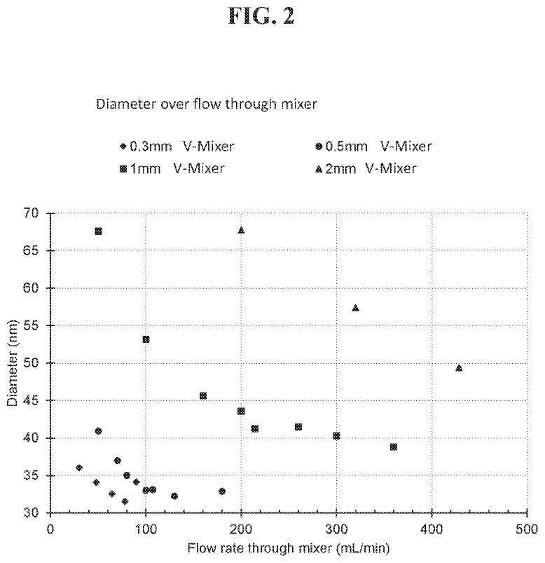

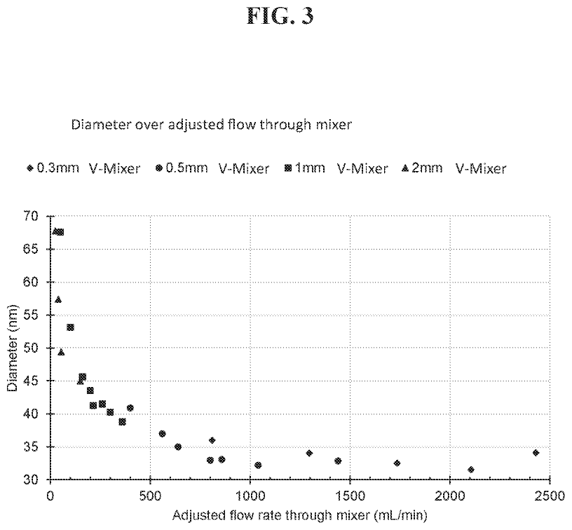

A clear eLNP size decrease with flow rate increase was observed, and larger mixers need larger flow rate to decrease the sizes of eLNP to mid-30 nm level, as shown in FIG. 2. A plateau of size was observed around 35 nm. For better analysis of size across different sizes of mixers, the concept of “adjusted flow rate” was introduced. “Adjusted flow rate” helps to compare size vs. flow rate across different sizes of mixers at the same level. The 1 mm mixer was taken as a baseline, so the adjusted flow rate for 1 mm mixer was the same as the real flow rate. For an “x” mm mixer, the adjusted flow rate=(1/x)³*real flow rate. For example, the real flow rate 100 mL/min of a 0.5 mm mixer gave (1/0.5)³*100, which was 800 mL/min. On the other hand, 100 mL/min of 2 mm mixer gave (1/2)³*100 which is 12.5 mL/min for adjusted flow rate. In this way, the mass flow rate per volume was kept the same across different sizes of the mixers. The size over adjusted flow rate for V-Mixer is shown in FIG. 3.

Ethanol drop time was simulated and calculated based on method introduced in section 2.4.1. Turbulent mixing is needed for efficiency mixing. For V-Mixer mixers, only mixing conditions with Reynolds number in the turbulent region (Re>3k) was used for ethanol drop time calculation. Reynolds number was calculated using Re=ρvL/μ, where ρ is density of the water-ethanol mixture (30% V ethanol here), v is liner velocity through the outlet, L the dimension of the mixing chamber, which is 5 times of the outlet length and is the dynamic viscosity of water-ethanol mixture. 0.5 mm V-Mixer at 50 mL/min total flow through the mixer gave Re of 4.8k. Hence ethanol drop time was only calculated for flow rate larger than 50 mL/min for the 0.5 mm V-Mixer.

The detailed size information for benchtop T-Mixer mixer nanoprecipitation is shown in Table 8.

TABLE 8
Benchtop nanoprecipitation using T-Mixer mixers sizing results

| | Mixer outlet | Total | | | Ethanol |
|---|---|---|---|---|---|
| Experiment | diameter | flow | Size | | drop time |
| No. | (mm) | (mL/min) | (nm) | PDI | (s) |
| 1 | 0.5 | 10 | 83.3 | 0.214 | |
| 2 | 0.5 | 20 | 53.8 | 0.403 | |
| 3 | 0.5 | 50 | 37.6 | 0.277 | 0.0005 |
| 4 | 0.5 | 100 | 30.0 | 0.139 | 0.0002 |
| 5 | 0.5 | 200 | 29.8 | 0.266 | 0.000125 |
| 6 | 1 | 10 | 91.5 | 0.248 | |
| 7 | 1 | 20 | 70.5 | 0.323 | |
| 8 | 1 | 50 | 51.3 | 0.264 | 0.0096 |
| 9 | 1 | 100 | 46.6 | 0.287 | 0.0082 |
| 10 | 1 | 200 | 36.2 | 0.254 | 0.0041 |
| 11 | 1.5 | 10 | 121.8 | 0.194 | |
| 12 | 1.5 | 20 | 81.0 | 0.230 | |
| 13 | 1.5 | 50 | 55.0 | 0.228 | 0.010725 |
| 14 | 1.5 | 100 | 40.3 | 0.176 | 0.004694 |
| 15 | 1.5 | 200 | 37.8 | 0.246 | 0.003312 |
| 16 | 2 | 10 | 117.0 | 0.239 | |
| 17 | 2 | 20 | 83.8 | 0.232 | |
| 18 | 2 | 50 | 57.4 | 0.196 | 0.0522 |
| 19 | 2 | 100 | 42.7 | 0.202 | 0.0230 |
| 20 | 2 | 200 | 34.9 | 0.235 | 0.0148 |

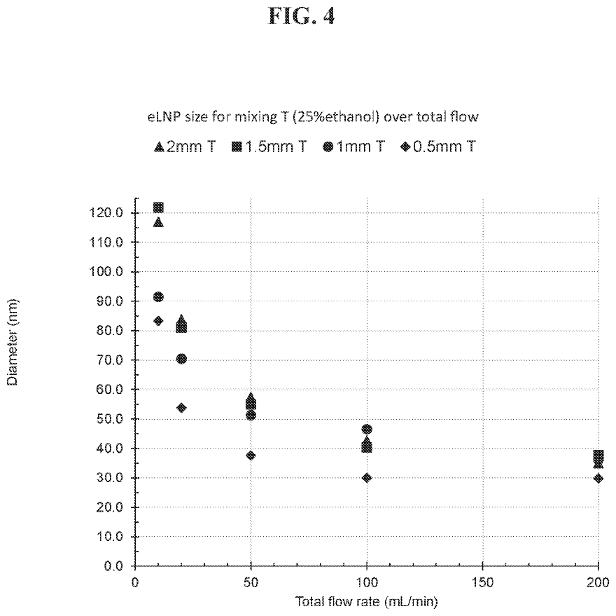

Nanoprecipitation using mixing T-Mixers was conducted using 25% LSS, so as to imitate GMP skid process. Similar to the trend observed from the benchtop V-Mixer experiments, decreasing size over increasing flow rate was observed for the T-Mixer nanoprecipitation, as shown in FIG. 4.

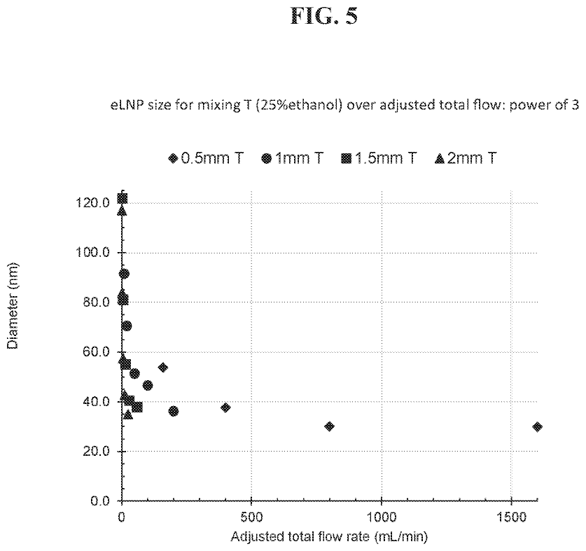

The same “adjusted flow rate” approach was used on experiment results of T-Mixer, where volumetric flow per volume was maintained the same, as 1 mm T-Mixer as the reference (adjusted flow=real flow for T-Mixer). Size of eLNP was plotted against adjusted flow through the T-Mixers in FIG. 5. Clearly the size of eLNPs made using T-Mixer drops much faster along the increase of adjusted flow rate compared to eLNPs made using V-Mixer.

Ethanol drop time was simulated and calculated based on method introduced in section 2.4.1. However, unlike V-Mixer, mixing T-Mixers do not have a clear mixing chamber, which makes the calculation of exact Reynolds number impossible for T-Mixer. To align with the conditions of V-Mixer, ethanol drop time was only calculated for mixing T-Mixer with flow rate larger than 50 mL/min.

Sizes of eLNPs Over Ethanol Drop Time as a Size Prediction Tool

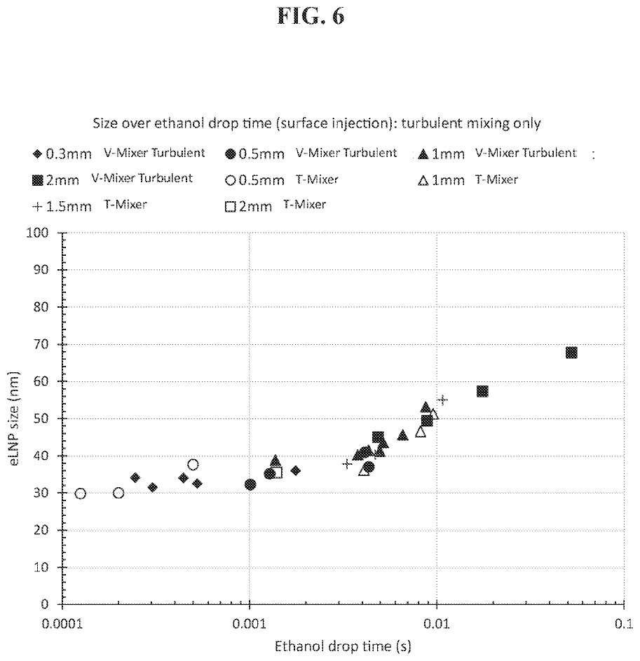

Sizes of eLNPs made using V-Mixer and T-Mixers in the turbulent flow region are plotted against ethanol drop time. One clear curve was observed, regardless mixing geometry of sizes used, as shown in FIG. 6.

This curve would be used as a prediction tool, so that the sizes of nanoparticles can be predicted based on ethanol drop time, calculated using a defined mixing flow rate and mixer type. Benchtop experiment size over ethanol drop time for 2 mm T mixer was not plot in FIG. 8, because significantly much smaller size was observed in that experiment, due to inaccurate machining of the T mixer. Instead, the size of eLNP produced in section 3.3 using a tri-clamp fitting 2 mm T mixer was plot against its ethanol drop time.

### 2 mm Mixer Skid Experiment Results

The sizes of eLNPs made using the GMP skid, with 2 mm V-Mixer, 1.5, 1.7 or 2 mm T-Mixer, with or without in-line static mixers are shown in Table 9. 1125 mL/min total flow rate and 25% vol LSS was used across all experiments.

TABLE 9
GMP skid scale experiments T-Mixer vs V-Mixer comparison

| | | | | | Size after TFF, | |
|---|---|---|---|---|---|---|
| | Ethanol | Aqueous | Size after | | sucrose addition | |
| | pressure | pressure | nanoprecipitation | | and filtration | |
| Mixer | (psi) | (psi) | (nm) | PDI | (in 72 hours) | PDI |
| 2 mm V-Mixer | 25 | 28 | 40.1 | 0.185 | 48.7 | 0.192 |
| 2 mm T-Mixer | 4 | 5 | 35.5 | 0.231 | 44.5 | 0.19 |
| 1.5 mm T-Mixer | 40 | 50 | 37.4 | 0.57 | NA | NA |
| 1.7 mm T-Mixer | 9.7 | 10.7 | 34.1 | 0.404 | NA | NA |
| 2 mm T-Mixer, | 1.9 | 5.7 | 35.8 | 0.214 | 44.2 | 0.31 |
| in-line mixer | | | | | | |
| 1.7 mm T-Mixer, | 2.5 | 9 | 32.4 | 0.301 | 40.4 | 0.298 |
| in-line mixer | | | | | | |

Experiment using 1.5 mm T-Mixer at 1125 mL/min total flow-through resulted in excessive pressure beyond what WM pumps could handle. Severe out-gasing was observed which created lots of air-water interface and therefore larger PDI. Comparing V-Mixer and T-Mixer of the same 2 mm outlet size, T-Mixer made smaller eLNPs at the same flow rate, with much smaller pressure at inlets. One explanation for this phenomenon is that the creation of vertex swirl in a V-Mixer converted mixing turbulence energy to wall shearing instead of fluid mixing, which led to slow mixing compared to impinging jets as well as larger pressure accumulation. Subsequent decrease of T-Mixer size led to even smaller size of eLNPs made. Similar sizes of eLNPs, but larger PDI was observed with the addition of in-line static mixer downstream to the mixing T-Mixer. Hence the addition of in-line mixer would not be recommended in future mixing T-Mixer experiments.

PHL loading experiments were conducted for eLNPs made using 2 mm V-Mixer, 2 mm T-Mixer, 1.7 mm T-Mixer with in-line static mixer and 2 mm T-Mixer with in-line static mixer. Each eLNP was loaded twice to ensure consistent results. Experiment protocol for PHL loading can be found in section 2.4.7. The results are shown in Table 10.

TABLE 10
PHL loading results for skid scale experiments

| | | | ILNP | |
|---|---|---|---|---|
| | eLNP | Days since | size after | |
| | size | nanopre- | neutralization | Encapsulation |
| Mixer | (nm) | cipitation | (nm) | efficiency |
| 2 mm V-Mixer | 50.1 | 11 | 121.2 | 96% |
| 2 mm V-Mixer | 50.1 | 11 | 122.5 | 95% |
| 2 mm T-Mixer | 46.1 | 11 | 106.1 | 96% |
| 2 mm T-Mixer | 46.1 | 11 | 106.7 | 96% |
| 1.7 mm T- | 41.9 | 6 | 91.4 | 96% |
| Mixer | | | | |
| inlinemix | | | | |
| 1.7 mm T- | 41.9 | 6 | 91.0 | 96% |
| Mixer | | | | |
| inlinemix | | | | |
| 2 mm T-Mixer | 43.5 | 6 | 102.0 | 96% |
| inlinemix | | | | |
| 2 mm T-Mixer | 43.5 | 6 | 100.0 | 96% |
| inlinemix | | | | |

Smaller sizes of eLNPs made using T-Mixer results in much smaller sizes of fLNP.

### Analytical and Characterization Tests for the Skid Experiments.

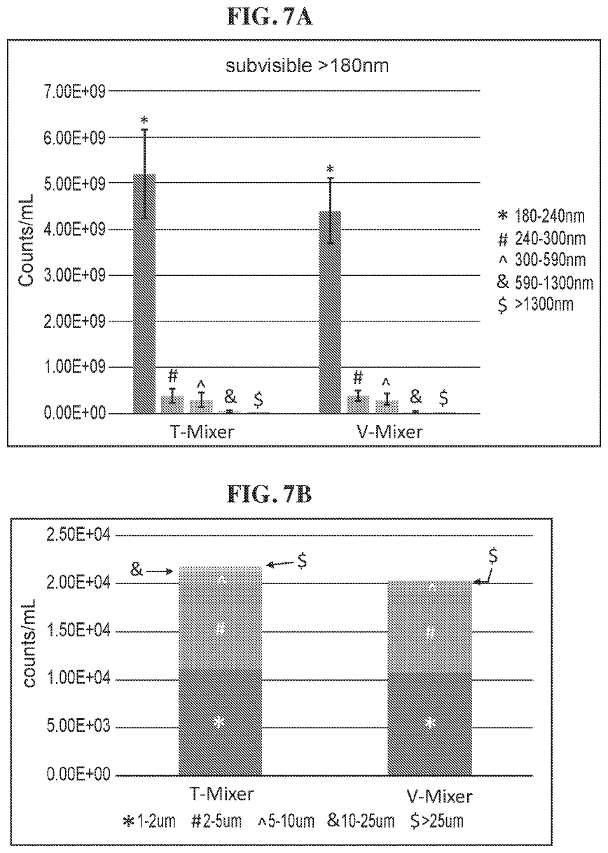

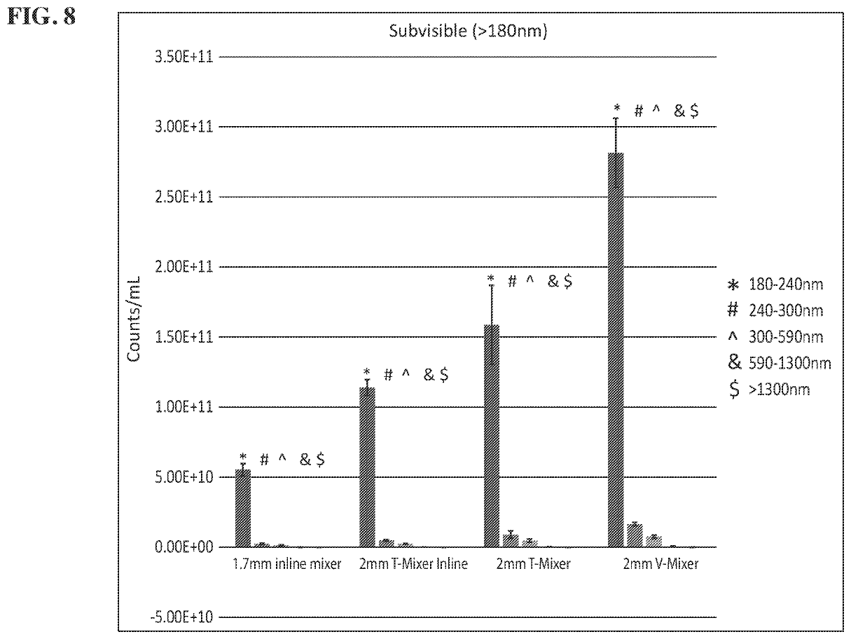

AF4 (detailed size distribution chromatography), flow cytometry (which looked at subvisible aggregates between 200 to 1000 nm) and flow cam (which looked at subvisible aggregates above 1 micron) results of the eLNP and fLNP made at skid level are shown in FIG. 7A, FIG. 7B, (eLNP) and FIG. 8 (fLNP).

No significant difference in subvisible aggregate counting was observed between 2 mm V-Mixer and T-Mixer, and size distribution peak of eLNPs made using T-Mixer is smaller than that by V-Mixer. Both evidence indicated that T-Mixer can be used to replace current V-Mixer on the skid scale.

No significant difference in subvisible aggregate counts were observed between fLNPs made using eLNPs from V-Mixer vs T-Mixers. The size distribution of fLNPs made from those T-Mixer-eLNPs is shifted to the smaller side. fLNPs made using V-Mixer-eLNPs also showed a larger “shoulder” peak, which corresponded to larger particle group.

### 3 and 4 mm Mixers Pilot Lab Mixing Level Results

Experiment result summary of the pilot lab 3 and 4 mm mixer nanoprecipitation is shown in Table 11.

TABLE 11
3 and 4 mm mixer scale-up experiment in the pilot lab results

| | | | Predicted | Predicted | Real | | | | | |
|---|---|---|---|---|---|---|---|---|---|---|
| | | Total | ethanol | water | Ethanol | Real | | Predicted | Real | |
| | | flow | pressure | pressure | pressure | water | EDT | size | size | |
| No. | Mixer | (mL/min) | (psi) | (psi) | (psi) | pressure | (s) | (nm) | (nm) | PDI |
| 1 | 4 mm | 4000 | 15 | 15 | 32 | 51 | 0.0158 | 55 | 54 | 0.189 |
| | V-Mixer | | | | | | | | | |
| 2 | 4 mm | 3600 | 12 | 12 | 29 | 41 | 0.0175 | 55 | 54 | 0.177 |
| | V-Mixer | | | | | | | | | |
| 3 | 4 mm | 3000 | 8 | 8 | 20 | 32 | 0.0207 | 60 | 58 | 0.200 |
| | V-Mixer | | | | | | | | | |
| 4 | 3 mm | 4000 | 10 | 13 | 9 | 12 | 0.0016 | 35 | 37 | 0.163 |
| | T-Mixer | | | | | | | | | |
| 5 | 3 mm | 5333 | 17 | 22 | 16 | 20 | 0.0013 | 35 | 33 | 0.150 |
| | T-Mixer | | | | | | | | | |
| 6 | 4 mm | 4000 | 3 | 6 | 8 | 11 | 0.0064 | 45 | 44 | 0.157 |
| | T-Mixer | | | | | | | | | |
| 7 | 4 mm | 5333 | 6 | 10 | 11 | 15 | 0.0048 | 40 | 38 | 0.178 |
| | T-Mixer | | | | | | | | | |

A fixed length of 6m for pre-ILD tubing is used (which gives 5s residence time for 5333 mL/min flow through the mixer), so there is variation in residence time based on flow rate through the mixer. Previous experiments show size of eLNP rises beyond 70 psi of pressure, hence 4 mm V-Mixer is operated to give minimum size already. Simulation gives <5% error in size prediction, <psi error for pressure prediction for T-Mixer, but 50/difference between prediction and measure pressure for V-Mixer. With the 5 times increase of flow rate through the mixer compared to the GMP skid level, there was a significant difference in terms of size of eLNPs made comparing 4 mm T-Mixer vs 4 mm V-Mixer. 3 mm T-Mixer made even smaller size, which is the same as those eLNP size using 0.5 mm mixers, despite the 100 times increase of flow rate.

Experiment 6 is Repeated to Make a 110L Batch, which Shows Consistent Size

Experiments using 3600 mL/min total 4 mm V-Mixer, 5333 mL/min total, 3 mm T and 5333 mL/min total, 4 mm T-Mixer proceeded to TFF using a 0.02 m² membrane, at 80 mL/min feed, for an approximately 200 g/m² loading. The sucrose-added and clarified eLNPs were used for PHL loading, and the results are shown in Table 12.

TABLE 12
PHL: from eLNPs made using 3 and 4mm mixers at pilot lab scale

| | Size of eLNP | | | | |
|---|---|---|---|---|---|
| | after TFF, | | | | |
| | sucrose addition | | Neutralized | | |
| eLNP | and filtration | eLNP | fLNP size | ILNP | |
| Mixer | (nm) | PDI | (nm) | PDI | EE |
| 4 mm | 56 | 0.166 | 167 | 0.222 | 84% |
| V-Mixer | | | | | |
| 3 mm | 37 | 0.179 | 98 | 0.161 | 96% |
| T-Mixer | | | | | |
| 4 mm | 41 | 0.195 | 108 | 0.174 | 96% |
| T-Mixer | | | | | |

Much smaller sizes of eLNP made from T-Mixers resulted in much smaller sizes of fLNPs after PHL as well as higher encapsulation efficiency.

### 3.6 Analytical and Characterization Tests for the Pilot Lab Scale Experiments

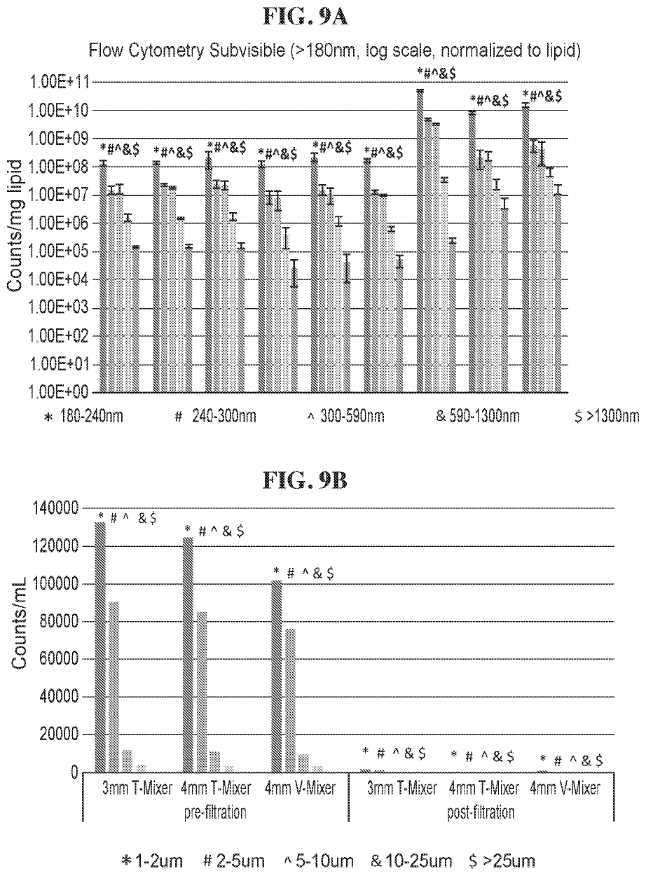

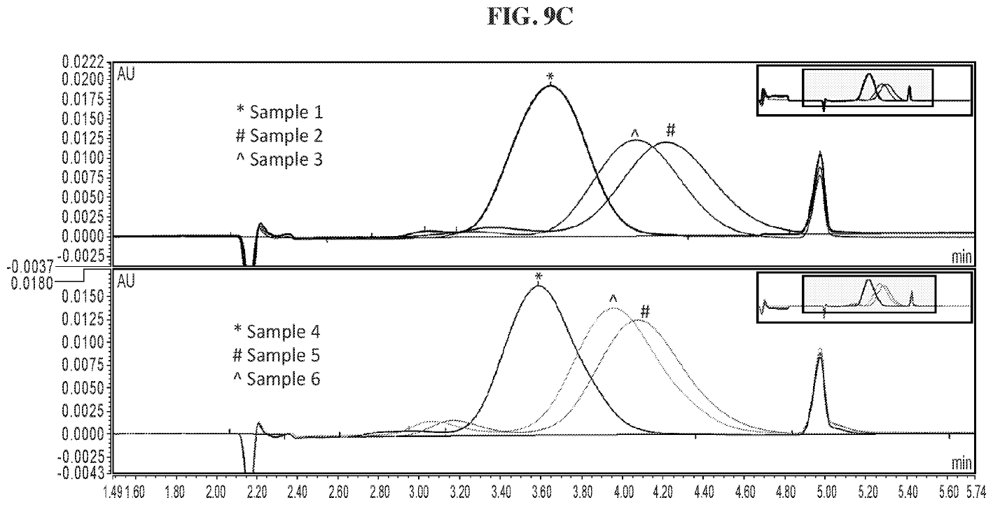

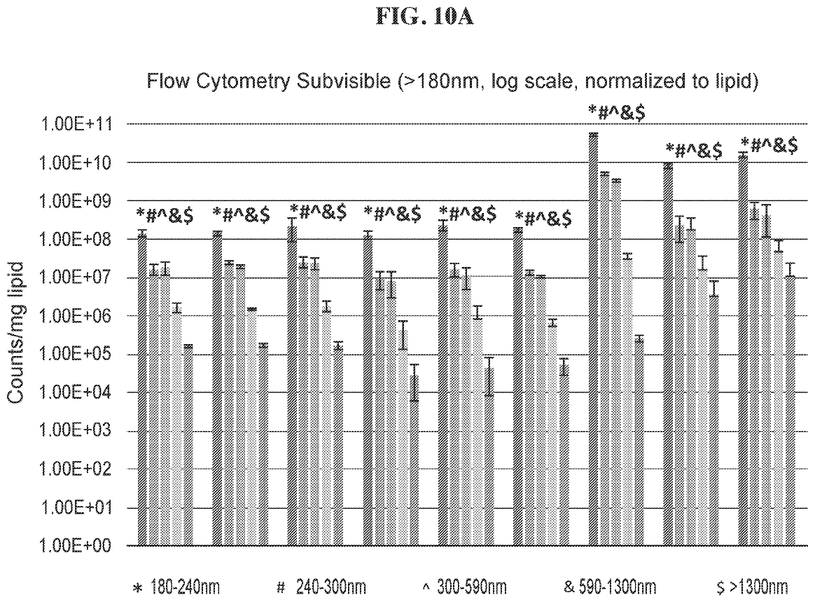

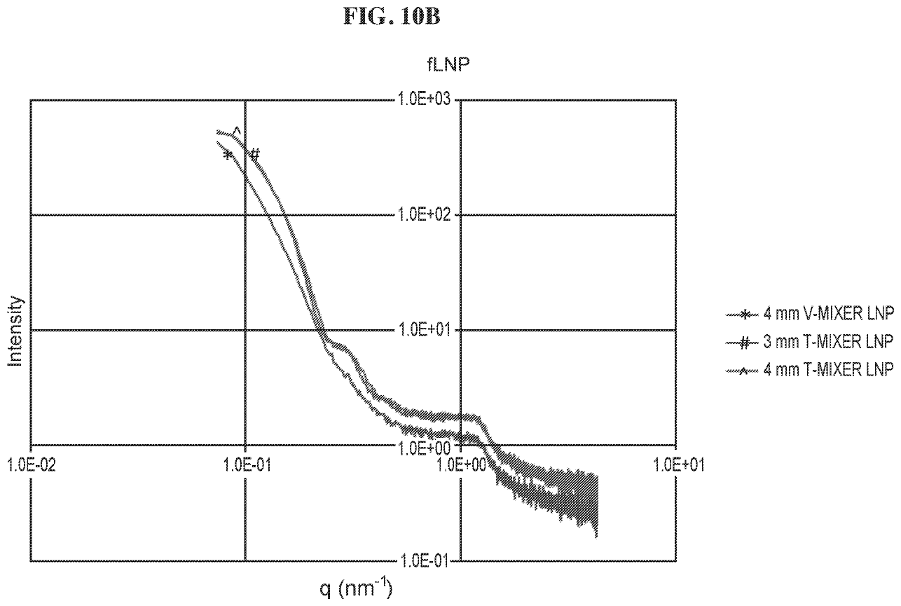

AF4 (detailed size distribution chromatography), CZE (charge over size differentiated chromatography), flow cytometry (which looked at subvisible aggregates between 200 to 1000 nm), flow cam (which looked at subvisible aggregates above 1 micron) and SAXS (looking at surface morphology and homogeneity) results of the eLNP and fLNP made at pilot lab level are shown in FIGS. 9A-9C (eLNP) and FIGS. 10A-10B (fLNP).

This series of studies, starting from benchtop smaller V-Mixer and T-Mixer connected to the LD-300 pumps, to the GMP skid level 2 mm V-Mixer vs T-Mixer and eventually to 3 and 4 mm mixers operated by the QF1200 pumps, demonstrated the scalability of using T-Mixer mixers across different production level. Whereas, the V-Mixer displayed larger eLNPs sizes at the same flow rate as T-Mixer. Given pressure upper limit of nanoprecipitation and the fact that T-Mixer also incurred much less pressure at the same flow rate compared to V-Mixer, T-Mixer mixers are better scale-up candidates in certain aspects.

### EQUIVALENTS

The details of one or more embodiments of the invention are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

We claim:

1. A process for preparing lipid nanoparticles (LNPs), comprising mixing a lipid solution with an aqueous buffer solution in a T-Mixer, thereby forming a lipid nanoparticle solution (LNP solution) comprising LNPs, wherein:
the lipid solution is fed to a lipid inlet of the T-Mixer at a lipid inlet back pressure of about 60 psi or less;
the aqueous buffer solution is fed to a buffer inlet of the T-Mixer at a buffer inlet back pressure of about 60 psi or less;
the LNP solution exits the LNP outlet of the T-Mixer at a flow rate of about 500 mL/min or greater; and
the LNPs have an average diameter of about 100 nm or less.

2. The process of claim 1, wherein:
the lipid inlet back pressure of the lipid solution in the T-Mixer is lower as compared to the lipid inlet back pressure of a comparable process using a V-Mixer;
the buffer inlet back pressure of the lipid solution in the T-Mixer is lower as compared to the buffer inlet back pressure of a comparable process using a V-Mixer;
the diameter of the T-Mixer is greater than the diameter of a V-Mixer used in a comparable process;
the LNP flow rate of the LNP solution exits the T-Mixer is greater than the LNP flow rate of a comparable process using a V-Mixer; and/or
the average diameter of the LNPs is less than the average diameter of the LNPs being prepared by a comparable process using a V-Mixer.

3. The process of claim 1, wherein the lipid solution is fed to a lipid inlet of the T-Mixer at a lipid inlet back pressure of about 10 psi or less, about 11 psi or less, about 12 psi or less, about 13 psi or less, about 14 psi or less, about 15 psi or less, about 16 psi or less, about 17 psi or less, about 18 psi or less, about 19 psi or less, about 20 psi or less, about 21 psi or less, about 22 psi or less, about 23 psi or less, about 24 psi or less, about 25 psi or less, about 26 psi or less, about 27 psi or less, about 28 psi or less, about 29 psi or less, about 30 psi or less, about 32 psi or less, about 34 psi or less, about 36 psi or less, about 38 psi or less, about 40 psi or less, about 42 psi or less, about 44 psi or less, about 46 psi or less, about 48 psi or less, about 50 psi or less, about 52 psi or less, about 54 psi or less, about 56 psi or less, or about 58 psi or less.

4. The process of claim 1, wherein the lipid solution is fed to a lipid inlet of the T-Mixer at a lipid inlet back pressure of about 10 psi, about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 32 psi, about 34 psi, about 36 psi, about 38 psi, about 40 psi, about 42 psi, about 44 psi, about 46 psi, about 48 psi, about 50 psi, about 52 psi, about 54 psi, about 56 psi, or about 58 psi, or about 60 psi.

5. The process of any one of claims 1-4, wherein the aqueous buffer solution is fed to an aqueous buffer inlet of the T-Mixer at a lipid inlet back pressure of about 10 psi or less, about 11 psi or less, about 12 psi or less, about 13 psi or less, about 14 psi or less, about 15 psi or less, about 16 psi or less, about 17 psi or less, about 18 psi or less, about 19 psi or less, about 20 psi or less, about 21 psi or less, about 22 psi or less, about 23 psi or less, about 24 psi or less, about 25 psi or less, about 26 psi or less, about 27 psi or less, about 28 psi or less, about 29 psi or less, about 30 psi or less, about 32 psi or less, about 34 psi or less, about 36 psi or less, about 38 psi or less, about 40 psi or less, about 42 psi or less, about 44 psi or less, about 46 psi or less, about 48 psi or less, about 50 psi or less, about 52 psi or less, about 54 psi or less, about 56 psi or less, or about 58 psi or less.

6. The process of any one of claims 1-4, wherein the aqueous buffer solution is fed to a aqueous buffer inlet of the T-Mixer at a lipid inlet back pressure of about 10 psi, about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 32 psi, about 34 psi, about 36 psi, about 38 psi, about 40 psi, about 42 psi, about 44 psi, about 46 psi, about 48 psi, about 50 psi, about 52 psi, about 54 psi, about 56 psi, or about 58 psi, or about 60 psi.

7. The process of any one of claims 1-6, wherein the LNP solution exits the LNP outlet of the T-Mixer at a flow rate of about 500 mL/min or greater, about 550 mL/min or greater, about 600 mL/min or greater, about 650 mL/min or greater, about 700 mL/min or greater, about 750 mL/min or greater, about 800 mL/min or greater, about 850 mL/min or greater, about 900 mL/min or greater, or about 950 mL/min or greater.

8. The process of any one of claims 1-6, wherein the LNP solution exits the LNP outlet of the T-Mixer at a flow rate of about 1000 mL/min or less, about 950 mL/min or less, about 900 mL/min or less, about 850 mL/min or less, about 800 mL/min or less, about 750 mL/min or less, about 700 mL/min or less, about 650 mL/min or less, about 600 mL/min or less, or about 550 mL/min or less.

9. The process of any one of claims 1-6, wherein the LNP solution exits the LNP outlet of the T-Mixer at a flow rate of about 500 mL/min, about 520 mL/min, about 540 mL/min, about 560 mL/min, about 580 mL/min, about 600 mL/min, about 620 mL/min, about 640 mL/min, about 660 mL/min, about 680 mL/min, about 700 mL/min, about 720 mL/min, about 740 mL/min, about 760 mL/min, about 780 mL/min, about 800 mL/min, about 820 mL/min, about 840 mL/min, about 860 mL/min, about 880 mL/min, about 900 mL/min, about 920 mL/min, about 940 mL/min, about 960 mL/min, about 980 mL/min, or about 1000 mL/min.

10. The process of any one of claims 1-9, wherein the LNPs have an average diameter of about 95 nm or less, about 90 nm or less, about 85 nm or less, about 80 nm or less, about 75 nm or less, about 70 nm or less, about 65 nm or less, about 60 nm or less, about 55 nm or less, about 50 nm or less, about 45 nm or less, about 40 nm or less, about 35 nm or less, about 30 nm or less, about 25 nm or less, about 20 nm or less, or about 15 nm or less.

11. The process of any one of claims 1-9, wherein the LNPs have an average diameter of about 10±5 nm, about 15±5 nm, about 20±5 nm, about 25±5 nm, about 30±5 nm, about 35±5 nm, about 40±5 nm, about 45±5 nm, about 50±5 nm, about 55±5 nm, about 60±5 nm, about 65±5 nm, about 70±5 nm, about 75±5 nm, about 80±5 nm, about 85±5 nm, about 90±5 nm, about 95±5 nm, or about 100±5 nm.

12. The process of any one of claims 1-9, wherein the lipid nanoparticles have an average diameter of about 30±5 nm, about 35±5 nm, or about 40±5 nm.

13. The process of any one of claims 1-12, wherein upon mixing, the lipid solution and the aqueous buffer solution form a transient mixture, wherein the concentration of the organic solvent in the transient mixture reduces over an organic solvent drop time, thereby forming the LNP solution.

14. The process of claim 13, wherein the organic solvent drop time is about 0.5 second or less.

15. The process of claim 13, wherein the organic solvent drop time is about 0.45 second or less, about 0.4 second or less, about 0.35 second or less, about 0.3 second or less, about 0.25 second or less, about 0.2 second or less, about 0.15 second or less, about 0.1 second or less, about 0.09 second or less, about 0.08 second or less, about 0.07 second or less, about 0.06 second or less, about 0.05 second or less, about 0.04 second or less, about 0.03 second or less, about 0.02 second or less, or about 0.01 second or less.

16. The process of claim 13, wherein the organic solvent drop time is about 0.1 second or less, about 0.09 second or less, about 0.08 second or less, about 0.07 second or less, about 0.06 second or less, about 0.05 second or less, about 0.04 second or less, about 0.03 second or less, about 0.02 second or less, or about 0.01 second or less.

17. The process of any one of claims 1-16, wherein the lipid solution further comprises a structural lipid, a phospholipid, and an organic solvent.

18. The process of claim 17, wherein the organic solvent is a C₁-C₆ alcohol.

19. The process of claim 18, wherein the C₁-C₆ alcohol is ethanol.

20. The process of any one of claims 1-19, wherein the lipid solution further comprises a PEG lipid.

21. The process of any one of claims 1-20, wherein the aqueous buffer solution comprises a buffering agent.

22. A lipid nanoparticle solution prepared by the process of any one of claims 1-21.

23. A lipid nanoparticle being prepared by the process of any one of claims 1-21.
